(19) United States
(12) Patent Application Publication
(10) Pub. No.: US 2022/0283165 A1
(43) Pub. Date: Sep. 8, 2022

(54) Methods of Enhancing Immunogenicity of Cancers

(71) Applicant: The Board of Trustees of the Leland Stanford Junior University, Stanford, CA (US)

(72) Inventors: Dean Felsher, San Mateo, CA (US); Carolyn R. Bertozzi, Menlo Park, CA (US); Anja Deutzmann, Redwood City, CA (US); Benjamin Smith, Redwood City, CA (US); Simon Wisnovsky, Redwood City, CA (US)

(21) Appl. No.: 17/625,264

(22) PCT Filed: Jul. 10, 2020
(86) PCT No.: PCT/US2020/041603

§ 371 Date: Jan. 6, 2022

Related U.S. Application Data
(60) Provisional application No. 62/873,727, filed on Jul. 12, 2019.

(51) Int. Cl. G01N 33/574; C07K 16/28

(52) U.S. Cl. CPC G01N 33/57426; C07K 16/2896; G01N 33/57492; C07K 2317/76; G01N 2333/82; G01N 2333/91091

(57) ABSTRACT

Provided are methods of enhancing immunogenicity of cancers. In certain aspects, the methods include administering an effective amount of a sialic acid modulator to an individual identified as having a cancer comprising dysregulated Myc. According to some aspects, the methods include administering an effective amount of a disialyl-T modulator to an individual identified as having a cancer comprising cell surface expression of disialyl-T. In certain aspects, the methods include administering an effective amount of an agent to an individual identified as having a cancer comprising cell surface expression of a glycoprotein comprising an O-glycosylated mucin-like domain, where the agent modulates the glycoprotein. Also provided are methods of assessing whether a cancer of an individual comprises dysregulated Myc.

### CROSS-REFERENCE TO RELATED APPLICATIONS

This application claims the benefit of U.S. Provisional Patent Application No. 62/873,727, filed Jul. 12, 2019, which application is incorporated herein by reference in its entirety.

### STATEMENT OF GOVERNMENT SUPPORT

This invention was made with Government support under contracts CA188383 and GM059907 awarded by the National Institutes of Health. The Government has certain rights in the invention.

### INTRODUCTION

Tumor cells are selected for their ability to avoid destruction by the immune system. Cancer cells are disguised as “normal” cells by their ability to inactivate immune cells through secretion of signaling molecules or direct engagement of immune checkpoint molecules. A critical problem for immune oncology is the identification of novel immune checkpoint signaling pathways in order to interfere with cancer-cell mediated inactivation of the immune system. Most work to-date has focused on proteins. For instance, inactivation of the PD1/PDL1 axis is a common strategy to promote the anti-tumor immune response. Immunotherapies targeting these proteins can lead to activation of anti-tumor immunity and dramatic clinical responses. However, (1) resistance to PD1/PDL1 checkpoint inhibitors develops routinely, and (2) not all tumors respond to checkpoint inhibitor therapy.

Elevated display of the sugar sialic acid on the surface of tumor cells correlates with aggressive phenotypes. While glycans containing sialic acid (“sialosides”) have long been known to promote cell adhesion and motility, they have more recently been implicated in immune evasion by binding to Siglecs, a family of receptors on most leukocytes that carry intracellular signaling motifs capable of modulating immune cell function. Despite increasing recognition that binding of tumor cell sialosides to immune cell Siglecs is an important aspect of tumorigenesis, the determinants of sialoside synthesis in cancer are not well characterized.

### SUMMARY

Provided are methods of enhancing immunogenicity of cancers. In certain aspects, the methods include administering an effective amount of a sialic acid modulator to an individual identified as having a cancer comprising dysregulated Myc. According to some aspects, the methods include administering an effective amount of a disialyl-T modulator to an individual identified as having a cancer comprising cell surface expression of disialyl-T. In certain aspects, the methods include administering an effective amount of an agent to an individual identified as having a cancer comprising cell surface expression of a glycoprotein comprising an O-glycosylated mucin-like domain, where the agent modulates the glycoprotein. Also provided are methods of assessing whether a cancer of an individual comprises dysregulated Myc.

### BRIEF DESCRIPTION OF THE FIGURES

FIG. 1 Myc regulates sialoside display in leukemia. Panels A-D: O- and N-glycomics analyses of a murine T-acute lymphoblastic leukemia (T-ALL) cell line in the MYC high versus low states. Among all glycans, the most significant difference between the MYC high and MYC low states is in sialyl-T and disialyl-T glycans (highlighted). In the MYC high state, the relative ratio of disialyl-T to sialyl-T increases. In the T-ALL cell line used in this study, MYC expression was under the control of a tetracycline off system and could thus be controlled by doxycycline treatment. Panel E: No differences in total sialic acids in whole cell T-ALL lysate between the MYC high (wt) and low (dox) states. MYC expression was reduced and sialic acid species were quantified using a DMB assay. Panel F: Increased display of cell surface sialic acids in the MYC high versus low states. MYC expression was turned off for the indicated amount of time and sialic acids on the cell surface were detected using the PAL method. Panel G: Increased display of disialyl-T on T-ALL in the MYC high versus MYC low states. T-ALL cells were pre-treated with sialidase to remove all surface sialic acids, and recovery of glycan display proceeded in the MYC high versus low states. Cells were stained with the lectin MAL-II to detect disialyl-T. Panel H: Display of the sialoside ligands of Siglec-E on T-ALL are increased in the MYC high state. T-ALL cells were treated with doxycycline for 48 hours to reduce MYC expression and then probed for glycans that bind Siglec-E using recombinant Siglec-E—Fc protein.

FIG. 2 St6galnac4 synthesizes the Siglec ligand disialyl-T. Panel A: T-ALL cells express high levels of a human MYC transgene that may be turned off via doxycycline administration. RNA-sequencing was performed on T-ALL cells treated with doxycycline for 0, 2, 4, 8, or 24 hours. Panel B: T-ALL cells in the MYC high state are metabolically more active as revealed by a GO term analysis of genes significantly upregulated in the MYC high state. Panel C: Among the 20 sialyltransferases, St6galnac4 is the most significantly upregulated transcript in the MYC high state. Panel D: St6galnac4 expression is elevated in the MYC high state and decreases rapidly when MYC is turned off in T-ALL. Panel E: MYC expression in T-ALL may be controlled precisely using various doses of doxycycline. Western blot reveals that treatment of T-ALL with 500 pg/mL doxycycline is sufficient to turn off MYC expression and that 20 pg/mL achieves and intermediate MYC dose. Panel F: St6galnac4 expression levels correlate with MYC dose. T-ALL cells were treated with the indicated concentration of doxycycline to turn off MYC expression for 48 hours prior to RNA-sequencing. Panel G: MYC binds to the St6galnac4 promoter as revealed by chromatin immunoprecipitation sequencing (ChIP-seq) in T-ALL. Panel H: The sialyltransferase ST6GALNAC4 elaborates the glycan sialyl-T into disialyl-T by attaching a sialic acid to GaINAc in an alpha2,6 linkage. Panel I: St6galnac4 synthesizes the glycan disialyl-T on T-ALL. O-glycomics analysis of St6galnac4 knockout cells reveals the near complete absence of disialyl-T. Panel J: St6galnac4 knockout T-ALL cells that lack disialyl-T display no ligands capable of binding Siglec-E. Either safe targeting control or St6galnac4 knockout cells were stained with Siglec-E—Fc to detect the presence of sialosides that interact with Siglec-E.

FIG. 3 MYC promotes Siglec-7 ligand display on human cancer. Panel A: The sialyltransferases St6galnac1 and St6galnac4 synthesize Siglec ligands on human cancer cell lines. Siglec-7 is the human homolog of Siglec-E. Sialyltransferase knockout or control human cells were stained for sialosides capable of interacting with the indicated Siglec by probing with recombinant Siglec-Fc protein. Panel B: ST6GALNAC4 synthesizes Siglec-7 ligands on the human CML cell lines K562s. Panels C and D: ST6GALNAC4 knockout K562s have significantly depressed ratios of disialyl-T to sialyl-T relative to control as revealed by O-glycomics. Panel E: The sialyltransferases ST6GALNAC1 and ST6GALNAC4 contribute to synthesis of the glycan disialyl-T. Because these sialyltransferases may compensate for one another, the effects of knocking out one transferase in a given cell line may be predicted by computing the relative expression ratio of ST6GALNAC4 to ST6GALNAC1. Raji cells have relatively more ST6GALNAC4, whereas K562s have relatively more ST6GALNAC1. Panel F: In Raji and Ramos cells that are enriched for ST6GALNAC4 compared to ST6GALNAC1, knocking out ST6GALNAC4 leads to complete removal of Siglec-7 ligands.

FIG. 4 The MYC oncogene controls display of checkpoint protein ligands such as CD47 and PD-L1. Display of these ligands confers tumors with a selective advantage by allowing them to inhibit immune cell activation. Interactions between tumor cell glycans and immune cell Siglecs are an emerging parallel axis of immunomodulation: glycans displayed by tumor cells that contain the sugar sialic acid (sialosides) bind to immune cell Siglecs to promote immune evasion. Demonstrated herein is that—inter alia—MYC acts through the sialyltransferase St6galnac4 to remodel glycans on the tumor cell surface and promote tumor growth.

FIG. 5A genome-wide CRISPRi screen identifies core drivers of cell-surface Siglec ligand expression. Panel A: Domain architecture of Siglec glyco-immune checkpoint receptors. Siglec receptors bind to sialylated glycans to inhibit activation of immune cells. Panel B: Cell surface staining of K562 cells with Siglec-7-Fc and Siglec-9-Fc chimeric proteins. Chimeras were precomplexed with Anti-hFc-AlexaFluor488 at 1 ug/ml for 1 hour, staining was performed on ice for 30 minutes and cells were analyzed by flow cytometry. Panel C: Workflow of genome-wide CRISPRi screen to identify genes involved in Siglec ligand biogenesis. 100×10⁶ CRISPRi library-infected K562 dCas9-KRAB cells were stained with Siglec-7-Fc and Siglec-9-Fc proteins. FACS was performed to isolate a population of cells exhibiting a 10fold reduction in cell surface staining. Panel D: Results of Siglec-7 and Siglec-9 CRISPRi screens. Genes ranked by positive selection score, where a higher score indicates a greater tendency for sgRNAs against that gene to be enriched in low Siglec-Fc staining cells. Panel E: GO analysis of screen hits. Analysis was performed using GOrilla to find GO terms enriched with a p value <10⁵. The top 10 terms for component, function and pathway analysis are shown ranked by fold enrichment. Panel F: Domain structure of CD43 (SPN), a top hit obtained in the Siglec-7 but not Siglec-9 genome-wide screen.

FIG. 6 CD43 is the major cell-surface glycoprotein ligand for Siglec-7. Panel A: Labeling intensity of CD43 KO cells generated by CRISPR-Cas9 editing with Siglec-7-Fc and SNA/MAH-11. Siglec-7-Fc labeling is reduced by CD43 knockout, while labeling with the general sialic acid-binding lectins MAH-11 and SNA is not. Mean fluorescence intensity over 3 independent replicates is indicated. Panel B: Enrichment of lysates with recombinant Siglec-Fc constructs. 5×10⁶ K562 cells were lysed in PBS with 0.1% NP-40 and enriched over beads coated with indicated Siglec-Fc chimeras. Interacting proteins were eluted off of the beads and immunoblotting for CD43 was subsequently performed. Panel C: Enrichment of lysates with recombinant Sig7-Fc followed by tryptic digest and MS analysis shows CD43 as the primary enriched cell surface protein. Identified proteins were ranked by combined spectral intensity of all peptides corresponding to that protein ID. Enrichment of proteins from untreated vs sialidase treated lysates was performed to identify proteins binding in a sialic acid-dependent manner. CD43 was identified at high intensity in Sig7-Fc pulldowns from untreated lysates but not from sialidase treated lysates.

FIG. 7 Siglec-7 binds a CD43 glycoform containing predominantly disialyl T glycan structures. Panel A: Pathway for synthesis of sialyl-3-T, sialyl-6-T and disialyl-T O-glycan structures. Bolded genes are glycosyltransferases recovered as significant hits from CRISPRi screen. Panel B: Sequential enzymatic digestion of CD43 with various glycan-degrading enzymes. Sia=VC-Sialidase, β-Gal=β-Galactosidase, O-Glyc=O-Glycosidase from S. Pneumoniae. The latter two enzymes act only on the unsubstituted core 1 structures indicated. Digestions were performed in K562 lysate and CD43 was detected by immunoblot with an antibody against the C-terminal domain. Panel C: Binding of PNA lectin to CD43 treated with glycan-degrading enzymes. Panel D: O-glycomics analysis of CD43 performed by chemical beta-elimination of O-glycans and LC-MS. Panel E: MS/MS analysis of monosialylated glycan structures to determine linkage orientation of sialic acid residues. Panel F: Siglec7-Fc pulldown of CD43 treated with a mucin-specific protease (StcE) to minimally truncate the extracellular domain and VC-Sialidase (Sia) to desialylate the protein. Only full-length CD43 fragments interacted with Siig7-Fc, indicating a binding site close to the N-terminal end of the extracellular domain.

FIG. 8 The Siglec-7/CD43 interaction suppresses activation of cytotoxic immune cells. Panel A: Siglec-7-Fc enrichment of T cell and monocyte lysates isolated from primary human PBMCs. Enzyme digestions and CD43 immunoblotting was performed to identify discrete glycoforms bound by Siglec-7. Panel B: Siglec-7 enrichment of lysates prepared from a range of leukemia cell lines shows varying expression of Siglec-7-binding CD43 glycoforms. Panel C: NK cell killing of CD43 KO K562 cells at a 4:1 effector:target ratio. Panel D: Treatment of K562 cells with the Mem-59 anti-CD43 antibody reduces Siglec-7-Fc staining. Panel E: NK cell killing of CD43-expressing leukemic cells following treatment of leukemia cell lines with MEM-59, an anti-CD43 antibody that binds sialylated structures near the CD43 N-terminus. All error bars indicate SEM. *p<0.05, **p<0.01, ***p<0.001.

FIG. 9 ST6GALNAC4 synthesizes Siglec-E/-7 ligands. Panel A: Heatmap of Siglec binding capacity displayed by control or St6galnac4^{-/31} T-ALL as measured by flow cytometry following staining with the indicated Siglec-Fc reagent. Cells were treated with either PBS or doxycycline for 48 hours to turn off the MYC transgene. Siglec-Fc reagents are indicated on the right side of the plot, where “m” indicates murine and “h” indicates human. Bold/red font highlights substantial reductions in presentation of ligands for human Siglec-7 and murine Siglec-E on St6galnac4^{-/-} and MYC off cells. Plot is representative of two independent experiments. Panels B-C: Display of ligands for Siglec-7 (B) and Siglec-E (C) on murine T-ALL at the indicated time points after turning MYC off as quantified by flow cytometry following staining with a Siglec-Fc reagent (n=3 per time point, two-tailed Student's t-test comparing each time point to t=0 hrs. *p<0.05, **p<0.01, data are representative of two independent experiments). Data are normalized to control, mean±s.d. Panels D and E: Representative flow cytometry plots of Siglec-7 (D) and Siglec-E (E) ligands displayed by WT and St6galnac4^{-/-} cells re-expressing wild type (WT) or mutant (Mut) St6galnac4. Plots are representative of three independent experiments. Panel F: Phagocytosis by BMDMs of WT and St6galnac4^{-/-} T-ALL rescued by transfection with empty vector or WT St6galnac4. (n=6 per group, two-tailed Student's t-test). Data are normalized to control (WT, no rescue) and presented as mean ±s.d. Panel G: Phagocytosis by Siglece^{-/-} BMDMs of WT and St6galnac4^{-/-} T-ALL rescued by transfection with empty vector or WT St6galnac4 (n=6 per group, two-tailed Student's t-test). Data are normalized to control (WT target cells, WT BMDMs, no rescue) and presented as mean±s.d. Panel H: Structure of the Siglec-E inhibitor. Panel I: Inhibition of Siglec-E-Fc binding to murine T-ALL by the Siglec-E inhibitor. Plot representative of three independent experiments. Panel J: Phagocytosis by BMDMs of control and St6galnac4^{-/-} T-ALL in the presence of Siglec-E inhibitor or mock DMSO treatment (n=6 per group, two-tailed Student's t-test). Data are normalized to control (mock treated WT target cells) and presented as mean±s.d.

FIG. 10 St6galnac4 promotes tumor growth in vivo. Panel A: Bioluminescence imaging of syngeneic wild type FVB/N mice transplanted IV with luciferase-labeled MYC-driven T-ALL cells expressing either a St6galnac4-specific or a control shRNA. Images show tumor burden on day nine and twenty-two post transplantation. Panel B: Tumor growth in mice described in (a) was assessed by bioluminescence imaging over time and quantified (n(Control)=11, n(sh-St6galnac4^{-/-})=9, Mixed Effects analysis). Panel C: Bioluminescence imaging of NSG mice transplanted IV with luciferase-labeled MYC-driven T-ALL cells expressing either a St6ga/nac4-specific or a control shRNA. Images show tumor burden on day two and fourteen post transplantation. Panel D: Tumor growth was assessed by bioluminescence imaging over time and quantified (n(Control)=7, n(sh-St6galnac4^{-/-})=8, Mixed Effects analysis). Panels E, F and G: Flow cytometric analysis of splenocytes isolated from NSG mice 14 days after IV-transplantation of MYC-driven T-ALL expressing either an St6ga/nac4-specific or a control shRNA (as shown in (c), right). Representative flow plots (left) and quantification of signal (right) are shown (n=5 per group, two-tailed Welch's t-test). Data summarized with box and whisker plots. (e) Tumor burden was assessed by CD4/CD8 double positivity. (f) Frequency of myeloid cells was determined by CD11 b positivity in the non-tumor population. (g) Expression of CD11b was assessed in non-tumor splenocytes. Median intensity of CD11b signal of CD11b-positive cells (gated on CD11b-FSC as shown in (f)) was determined. Each trace represents a different mouse. Panel H: Histopathologic appearance of splenic T-ALL in NSG mice as described in (c). Scale bar=50 μm. Multinucleated giant cells (MGCs) were quantified in 5 high power fields (HPF) per sample (n=5, two-tailed Mann-Whitney U test). Data summarized with box and whisker plots. Panel I: Volcano plots displaying glycogenes in MYC-driven mouse models of Burkitt's lymphoma (Ep-Myc) and T-ALL (EpSRa-tTA/tet-O-MYC). Panel J: Overlap analysis of differentially expressed glycogenes (at least 2-fold change with FDR<0.05) from (i) (glycogenes up and down: OR=4.91, p=9.04×10⁻⁷) in mouse models of Burkitt's lymphoma (Ep-Myc) and T-ALL (EpSRa-tTA/tet-O-MYC). Panel K: Heat map representation of glycogenes that are differentially expressed in splenocytes isolated from EpSRa-tTA/tet-O-MYC (MYC on/off) and normal (FVB/N) mice. Expression in normal tissue, in MYC on tumor tissue, and upon MYC inactivation-induced tumor regression (MYC off) is shown (n=3 per treatment group).

FIG. 11 MYC promotes Siglec-7 ligand display on human cancer. Panel A:

Summarized gene expression of MYC and ST6GALNAC4 in human T-ALL and peripheral blood mononuclear cells (PBMCs). Data from GSE62156, GSE27562, and GSE49515 were collated, and absolute expression was determined with Gene Expression Commons (n(tumor)=65, n(control)=45, two-tailed Mann-Whitney U test). Boxplot shows data quartiles. Panel B: Siglec-7 ligands on PEER cells following either knockout of

ST6GALNAC4 or pharmacologic MYC inhibition for 48 hours by incubation with 100 μM 10058-F4 (n(control)=8, n(KO)=4, n(inhibitor)=6, two-tailed Mann-Whitney U test). Data presented as mean ±s.d. Panel C: Representative Western blot for ST6GALNAC4 in PEER cells following MYC inhibition with 100 μM 10058-F4 for 48 hours. Data representative of two independent experiments. Panel D: Workflow to collect primary T-ALL from patients, treat with a MYC inhibitor (10058-F4), and quantify Siglec-7 ligands. Panel E: Siglec-7 ligands on patient T-ALL liquid biopsies treated with the indicated concentration of 10058-F4 for 48 hours. Data represent three donors. Panel F: Correlation of ST6GALNAC4 and MYC mRNA (RNA-seq) gene expression across all hematopoietic tumor samples (n=106) in the CCLE with MYC expression>3. Correlation determined by least squares regression. Panel G: Survival Z-score heatmap for each sialyltransferase across each hematopoietic tumor within the PRECOG database. Larger positive Z-Scores (red) indicate that patients with higher expression of the indicated gene exhibit reduced survival. Panel H: Siglec-7 ligand display by KARPAS-422 DLBCL cells following treatment with 10058-F4 for 48 hours (n=3 per group, two-tailed Student's t-test). Data presented as mean±s.d. Panel I: Survival stratified by median MYC and ST6GALNAC4 expression in a cohort of DLBCL patients (GSE4475). HR, hazard ratio from Cox Proportional Hazards model. Panel J: Venn diagram of patients in the same DLBCL cohort, showing individuals that fall into MYC high and ST6GALNAC4 high (greater than median expression) cohorts (X²-test for independence). Panel K: Pan-cancer overall survival analysis of TCGA data stratifying patients by median MYC and ST6GALNAC4 expression (n=2376 per group). HR, hazard ratio from Cox Proportional Hazards model. Panel L: Pan-cancer immune phenotype of TCGA tumors stratified by k-means clustering based on MYC and ST6GALNAC4 expression. Monocyte prevalence was calculated as a fraction of all lymphocytes. Data presented as mean±s.e.m. Panel M: Model for MYC-driven display of disialyl-T and regulation of the immune response via Siglec engagement.

### DETAILED DESCRIPTION

Before the methods of the present disclosure are described in greater detail, it is to be understood that the methods are not limited to particular embodiments described, as such may, of course, vary. It is also to be understood that the terminology used herein is for the purpose of describing particular embodiments only, and is not intended to be limiting, since the scope of the methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods. The upper and lower limits of these smaller ranges may independently be included in the smaller ranges and are also encompassed within the methods, subject to any specifically excluded limit in the stated range. Where the stated range includes one or both of the limits, ranges excluding either or both of those included limits are also included in the methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

The present disclosure provides methods of enhancing immunogenicity of cancers. In certain aspects, the cancers comprise dysregulated Myc and the methods comprise administering an effective amount of a sialic acid modulator to an individual identified as having a cancer comprising dysregulated Myc. The methods are based in part on the inventors' findings presented in the Experimental section herein that Myc acts through the sialyltransferase St6galnac4 to remodel glycans on the tumor cell surface and promote tumor growth. Details regarding the methods of the present disclosure will now be described.

Myc (UniProtKB-P01106 (human); UniProtKB-P01108 (mouse)) expression is tightly controlled under normal circumstances, yet Myc is frequently dysregulated in human cancers. Excess Myc expression can be induced upon retroviral promoter insertion, chromosomal translocation/amplification, activation of super-enhancers within the MYC gene, mutation or activation of upstream signaling pathways that enhance Myc stability or promote Myc expression, etc.

The gene encoding Myc, refseq NG_007161 on chromosome 8, encodes the proto-oncogene, which is a nuclear phosphoprotein that plays a role in cell cycle progression, apoptosis and cellular transformation. The encoded protein forms a heterodimer with the related transcription factor MAX. This complex binds to the E box DMA consensus sequence and regulates the transcription of specific target genes. Amplification of this gene is frequently observed in numerous human cancers. Translocations involving this gene are associated with Burkitt lymphoma and multiple myeloma in human patients. There is evidence to show that translation initiates both from an upstream, in-frame non-AUG (CUG) and a downstream AUG start site, resulting in the production of two isoforms with distinct N-termini. See, for example, NP_001341799 for the protein sequence; and Hann et al. (1988) Cell 52 (2), 185-195.

The Myc oncogene drives the pathogenesis of many hematopoietic malignancies, including Burkitt's lymphoma (BL), Diffuse large B cell lymphoma (DLBCL) and Acute Lymphoblastic Leukemia (ALL). These malignancies are often “oncogene-addicted” to MYC. Treating subjects having cancers that are specifically identified as MYC-addicted with new therapeutic modalities circumvents the issues associated with the undruggability of MYC.

As used herein, a “cancer” comprises one or more cancer cells, where by “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a primary tumor, a metastatic tumor, and the like.

By “immunogenicity” is meant the ability of cancer cells of a cancer to provoke an immune response (e.g., cell-mediated immune response) in the body of a human and other animal. As used herein, “enhancing immunogenicity” means that administration of the sialic acid modulator increases the immunogenicity of cancer cells of a cancer as compared to the immunogenicity of the cancer cells in the absence of administration of the sialic acid modulator.

As used herein, “a cancer comprising dysregulated Myc” is a cancer comprising cancer cells that exhibit increased Myc activity as compared to non-cancerous cells of the corresponding cell type, e.g., cancerous T cells that exhibit increased Myc activity as compared to the corresponding non-cancerous T cells. Increased Myc activity may be caused by various mechanisms, including but not limited to genomic amplification, transcriptional or translational activation due to upstream regulators, post-translational modifications leading to increased stability or activity, the absence of normal regulatory mechanisms, or decreased responsiveness to regulation. Increased Myc activity may be characterized by increased expression of Myc target genes, decreased expression of other Myc target genes, or by increased Myc occupancy on gene promoters. Expression changes of Myc target genes may depend on the extent of Myc dysregulation and may be tissue specific. In some embodiments the cancer is Myc-driven, e.g., causally dependent on Myc as a result of, for example, over-expression of Myc, constitutive expression of Myc, chromosomal translocation resulting in overactive Myc, and the like.

By “sialic acid modulator” is meant an agent that reduces the abundance of Myc-promoted sialic acid on the surface of cells in the individual, including (or exclusively or substantially exclusively) cancer cells of the individual, as compared to the abundance in the absence of the sialic acid modulator. The sialic acid modulator will vary depending upon the selected mechanism for reducing the abundance of Myc-promoted sialic acid on the surface of the cells. Non-limiting examples of how a sialic acid modulator may reduce the abundance of Myc-promoted sialic acid on the surface of cells include: inhibiting Myc; inhibiting expression or activity of a member of the sialic acid biosynthesis pathway; inhibiting sialic acid synthesis; hydrolyzing terminal sialic acids from glycoproteins or glycolipids; and the like.

According to some embodiments, the sialic acid modulator is a Myc inhibitor. The

Myc inhibitor may be any known or subsequently developed Myc inhibitor. In some embodiments, the Myc inhibitor targets Myc transcription. Myc transcription may be targeted by interfering with chromatin-dependent signal transduction to RNA polymerase, a process in which BRD4 (a member of the mammalian bromodomain and extraterminal (BET) family) has been implicated. MYC transcription is under BRD4 regulation. JQ1, an inhibitor of BRD4, competes with BRD4 for binding to acetylated lysines and displaces BRD4 from the super-enhancers within the MYC oncogene. As such, inhibition of the BET bromodomain with JQ1 showed potent anti-cancer effects both in vitro and in vivo in multiple hematopoietic cancers and pancreatic ductal adenocarcinoma (PDAC) exhibiting C-MYC overexpression. Neuroblastomas and other MYCN-driven cancers are also sensitive to BET inhibitors. GSK525762, a specific BET inhibitor, is currently in clinical trials for treating these hematopoietic malignancies and solid tumors (ClinicalTrials.gov: NCT01943851, NCT03266159). Also with respect to targeting Myc transcription, a general feature of MYC deregulation is its transcriptional regulation by Super-Enhancers (SEs), clusters of enhancers that are densely occupied by transcription factors and chromatin regulators, including CDK7 and CDK9, rendering this group of kinases targets for blocking Myc-dependent transcriptional amplification. Inhibition of CDK7 and/or CDK9 substantially reduces MYC expression, in turn resulting in widespread transcriptional downregulation of Myc target genes. Administration of specific inhibitors against CDK7 (THZ1) and/or CDK9 (PC585) induced potent anti-tumor effects in MYC-overexpressing T-cell acute lymphoblastic leukemia, mixed-lineage leukemia, neuroblastomas, and small cell lung cancers, validating these newly developed transcriptional CDK inhibitors as a treatment strategy that targets global transcriptional amplification in Myc-driven or Myc-promoted cancers. Accordingly, in some embodiments, the Myc inhibitor targets Myc transcription and is BET inhibitor (non-limiting examples of which include JQ1, GSK525762, and the like) or a CDK7 and/or CDK9 inhibitor (non-limiting examples of which include THZ1 and PC585, respectively).

In certain embodiments, a Myc inhibitor targets Myc translation. For example, pharmacological inhibition of the P13K/AKT/mTOR pathway (involved in regulation of translation initiation factor elF4E) markedly decreases Myc level and exhibits therapeutic efficacy in Myc-driven cancers, including neuroblastoma, small-cell lung carcinoma, breast cancer, and multiple hematopoietic cancers. Also with respect to targeting Myc translation, CPEB recruits Cat 1 deadenylase through an interaction with Tob, an antiproliferative protein, and inhibits c-Myc expression by accelerating the deadenylation and decay of its mRNA. As such, pharmacological approaches aimed at reactivating CPEB expression leads to inhibition of Myc translation. Accordingly, in some embodiments, the Myc inhibitor targets Myc translation and is a PI3K/AKT/mTOR pathway inhibitor (non-limiting examples of which include CCI-779, NVP-BEZ235, etc.) or a CPEB upregulator.

According to some embodiments, a Myc inhibitor targets Myc stability, which is tightly controlled by the ubiquitin-proteasome system. For example, the deubiquitinating enzymes USP28, USP36, and USP7 are involved in Myc stabilization and may be targeted to reduce the stability of Myc, thereby inhibiting Myc. For example, USP7 directly binds to and stabilizes N-Myc through deubiquitination in neuroblastomas cells, and a small-molecule inhibitor of USP7, P22077, markedly suppressed growth of MYCN-amplified neuroblastoma in a xenograft model. In addition, two AURKA inhibitors, MLN8054 and MLN8237, disrupt the Myc—AURKA complex, resulting in N-Myc degradation and tumor regression in MYCN-amplified neuroblastomas. MLN8237 also induced c-Myc degradation in P53-mutant human hepatocellular carcinoma cells. These data indicate the utility of AURKA inhibitors as therapeutics for the treatment of Myc-dependent cancers. Moreover, inhibitors of Polo-like kinase 1 (PLK1), such as B16727 or B12356, preferentially induce potent apoptosis of Myc-overexpressing tumor cells and synergistically potentiate the therapeutic efficacies of BCL-2 antagonists.

In certain embodiments, a Myc inhibitor targets the Myc-Max complex. The Myc-Max complex is required for the binding of Myc to DNA and its subsequent activation of target gene transcription. The peptide mimetic IIA6B17 is a small-molecule inhibitor of Myc-Max dimerization. The compound 10058-F4 is capable of disrupting the Myc-Max complex in HL60 cells. Another known inhibitor, Omomyc, a mutant basic helix-loop-helix peptide that sequesters Myc in a transcriptionally incompetent complex, prevents Myc-induced tumorigenesis in multiple mouse tumor models. Celastrol and celastrol-inspired triterpenoids have also been shown to directly inhibit Myc-Max complexes.

Further details regarding strategies for inhibiting Myc and specific Myc inhibitors which may be employed when practicing the methods of the present disclosure are provided, e.g., in Chen et al. (2018) Signal Transduction and Targeted Therapy 3:5; Fletcher et al. (2015) Biochim Biophys Acta 1849(5):525-543; and elsewhere.

According to some embodiments, the sialic acid modulator is an inhibitor of sialic acid biosynthesis. Sialic acids are substituents of glycan structures, appearing most frequently as the non-reducing terminal molecules of N-glycans, O-glycans, and glycosylphosphatidylinositol (GPI) anchored proteins. Eukaryotic systems and several prokaryotes express sialic acids, and other pathogenic bacteria, viruses and parasites utilize cell surface sialic acids as ligands as a means to adhere to cells, with the influenza viruses being the most well-known example of sialic-binding pathogens. Sialic acids are based on a cyclic nine-carbon structure with a carboxylic acid group at the Cl position. Sialic acids have an inherent negative charge on account of the carboxylic acid group. Common sialic acids include N-acetylneuraminic acid (Neu5Ac, NeuNAc or NANA), N-glycolylneuraminic acid (Neu5Gc), and N-acetyl-9-O-acetylneuraminic acid (Neu5,9Ac₂), and other species containing additional acetyl groups, sulfate groups, phosphate moieties, and/or the like.

Synthesis of sialic acids in animals takes place primarily in the cytosol and involves four enzymes participating in a five-step process. The dual activity enzyme UDP-GlcNAc 2-epimerase/ManNAc-6-kinase (gene symbol GNE) converts the starting substrate uridine 5′-diphosphate-N-acetyl-D-glucosamine (UDPGIcNAc) to N-acetyl-D-mannosamine (ManNAc) with removal of the UDP moiety and epimerization of the carbohydrate. The kinase function of the same enzyme phosphorylates the sugar to produce N-acetyl-d-mannose 6-phosphate (ManNAc-6-P). The condensation reaction between phosphoenol pyruvate (PEP) and ManNAc-6-P initiated by NeuAc-9-P-synthetase (gene symbol NANS) results in the phosphorylated sialic acid precursor N-acetylneuraminic acid 9-phopshate (Neu5Ac-9-P). This precursor is dephosphorylated by Neu5Ac-9-P-phosphatase (gene symbol NANP) to produce the key sialic acid N-acetylneuraminic acid (2-ketoacetamido-3,5-dideoxy-D-glycero-D-galactononulosonic acid; Neu5Ac). Modification to the neuraminic acid takes place in the Golgi, either before the carbohydrate moiety is transferred to a carbohydrate acceptor or after transfer. Sialic acid biosynthesis also includes the process of transferring a sialic acid to a glycoprotein or glycolipid, e.g., by a sialyltransferase enzyme.

In certain embodiments, when the sialic acid modulator is an inhibitor of sialic acid biosynthesis, the inhibitor of sialic acid biosynthesis is a sialyltransferase inhibitor. Sialyltransferases (STs) catalyze the transfer of sialic acid residues to non-reducing oligosaccharide chains of proteins and lipids, using cytidine monophosphate N-acetylneuraminic acid (CMP-Neu5Ac) as the donor. Non-limiting examples of sialyltransferase inhibitors that may be employed include sialic acid analogs, CMP-sialic acid analogs, cytidine analogs, oligosaccharide derivatives, aromatic compounds, flavonoids, lithocholic acid analogs (e.g., lithocholylglycine), and the like. Details regarding various sialyltransferase inhibitors which may be employed in the methods and conjugates of the present disclosure are found, e.g., in Wang et al. (2016) Biochim Biophys Acta. 1864(1):143-53; Szabo and Skropeta (2017) Medicinal Research Reviews 37(2):219-270; and elsewhere. In certain embodiments, the sialyltransferase inhibitor is a sialic acid analog, and the sialic acid analog is a fluorinated sialic acid analog. A non-limiting example of a fluorinated sialic acid analog is P-3Fₐₓ-Neu5Ac (3F-NeuAc)—a cell-permeable sialic acid analog that is converted to CMP-3F-NeuAc and inhibits all sialyltransferases.

According to some embodiments, when the sialic acid modulator is a sialyltransferase inhibitor, the sialyltransferase inhibitor inhibits a sialyltransferase upregulated by Myc dysregulation. For example, the sialyltransferase inhibitor may inhibit St6galnac4, demonstrated in the Experimental section herein to be sialyltransferase through which Myc acts to remodel glycans on the tumor cell surface and promote tumor growth. In certain embodiments, when the sialic acid modulator is a sialyltransferase inhibitor, the sialyltransferase inhibitor inhibits St6galnac1.

In certain embodiments, the sialic acid modulator is an enzyme. Examples of sialic acid modulating enzymes that may be employed include, but are not limited to, enzymes that hydrolyze terminal sialic acids from glycoproteins or glycolipids. By way of example, the enzyme may be a sialidase. Sialidases cleave the glycosidic linkages of sialic (neuraminic) acids, releasing terminal sialic acid residues from oligosaccharides, polysaccharides, glycoproteins, glycolipids, and other substrates.

Sialidases which may be employed include, but are not limited to, prokaryotic sialidases and eukaryotic sialidases. Prokaryotic sialidases that may be employed include bacterial sialidases. One example of a bacterial sialidase that may be employed is Salmonella typhimurium sialidase (e.g., UniProtKB-P29768). Another example of a bacterial sialidase that may be employed is Vibrio cholera sialidase (e.g., UniProtKB-P0C6E9). Eukaryotic sialidases that may be employed include, e.g., mammalian sialidases and non-mammalian eukaryotic sialidases. Mammalian sialidases (or mammalian neuraminidases) of interest include those from primates, e.g., human or non-human neuraminidases. In certain aspects, the sialidase is a human sialidase. According to certain embodiments, the human sialidase is selected from human neuraminidase 1 (e.g., UniProtKB-Q99519), human neuraminidase 2 (e.g., UniProtKB-Q9Y3R4), human neuraminidase 3 (e.g., UniProtKB-Q9UQ49), and human neuraminidase 4 (e.g., UniProtKB-Q8WWR8). It will be understood that the sialidase may be a derivative of any of the wild-type sialidases above, such as truncated derivatives, derivatives that include more or fewer amino acids than the corresponding wild-type sialidase, derivatives that include one or more amino acid substitutions (e.g., one or more conservative substitutions, one or more non-conservative substitutions, a substitution of a natural amino acid with a non-natural amino acid, and/or the like), etc. The derivatives are functional derivatives in that they retain at least a portion of the glycoside hydrolase activity of the parental wild-type sialidase.

According to some embodiments, the sialic acid modulator is a sialidase, and the sialidase is conjugated to a targeting moiety (e.g., an antibody) that specifically binds a molecule (e.g., tumor-specific or tumor-associated molecule) on the surface of the cancer cells such that the targeting moiety targets the sialidase to the cancer cells in the individual. Non-limiting examples of sialidase-targeting moiety conjugates that may be employed are described in, e.g., U.S. Ser. No. 16/308,732 and WO 2018/006034, the disclosures of which are incorporated herein by reference in their entireties for all purposes.

In certain embodiments, the sialic acid modulator inhibits expression or activity of a member of the sialic acid biosynthesis pathway. Members of the sialic acid biosynthesis pathway which may be targeted by the sialic acid modulator include, e.g., UDP-GlcNAc 2-Epimerase/ManNAc Kinase (GNE), NeuAc-9-P-synthetase (NANS), Neu5Ac-9-P-phosphatase (NANP), and cytidine monophosphate N-acetylneuraminic acid synthetase (CMAS). According to some embodiments, the sialic acid modulator is a small molecule that inhibits activity of a member of the sialic acid biosynthesis pathway. In certain embodiments, the sialic acid modulator is a nucleic acid that inhibits expression of a member of the sialic acid biosynthesis pathway. Non-limiting examples of nucleic acid-based inhibitors that may be employed include short interfering RNAs (siRNA), microRNAs (miRNA), morpholinos, and/or the like. Based on the available sequence information for the genes and transcripts of, e.g., GNE, NANS, NANP, and CMAS, suitable nucleic acid-based inhibitors may be designed, synthesized, and employed. For example, based on the available sequence information for the genes and transcripts that encode GNE, NANS, NANP, and CMAS, nucleic acid-based inhibitors such as siRNAs, miRNAs, morpholinos, etc. may be designed using available tools, e.g., siRNA Wizard from Invivogen, siDESIGN Center from Dharmacon, BLOCK-iT™ RNAi Designer from Invitrogen, miR-Synth available at microrna.osumc.edu/mir-synth, WMD3—Web MicroRNA Designer, a morpholino design tool provided by Gene Tools, etc. Approaches for designing and delivering siRNAs, miRNAs, morpholinos, etc. for targeting a particular mRNA are known and described, e.g., in Chakraborty et al. (2017) Mol Ther Nucleic Acids 8:132-143; Ahmadzada et al. (2018) Biophys Rev. 10(1):69-86; Zheng et al. (2018) Trends Biotechnol. 36(5):562-575; Mohanty et al. (2015) Curr Pharm Des. 21(31):4606-13; Gomes et al. (2015) Ageing Res Rev. 21:43-54; Gustincich et al. (2017) Prog Neurobiol. 155:194-211; Monsoori et al. (2014) Adv Pharm Bull. 4(4):313-321; and Xin et al. (2017) Mol Cancer 16:134.

According to some embodiments, the sialic acid modulator is a sialic acid binding agent. Non-limiting examples of sialic acid binding agents include, but are not limited to, antibodies, receptors, lectin-based sialic acid binding agents, and the like that bind (e.g., specifically bind) a particular sialic acid (e.g., disialyl-T) or class of sialic acids. The terms “antibody” and “immunoglobulin” include antibodies or immunoglobulins of any isotype (e.g., IgG (e.g., IgG1, IgG2, IgG3 or IgG4), IgE, IgD, IgA, IgM, etc.), whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to the sialic acid, including, but not limited to, Fv, single chain Fv (scFv), Fab, F(ab′)₂, Fab′, (scFv′)₂, and diabodies; chimeric antibodies; monoclonal antibodies, human antibodies, humanized antibodies (e.g., humanized whole antibodies, humanized antibody fragments, etc.); and fusion proteins including an antigen-binding portion of an antibody and a non-antibody protein or fragment thereof, e.g., an antibody Fc region or fragment thereof. The antibodies may be detectably labeled, e.g., with an in vivo imaging agent, or the like. The antibodies may be further conjugated to other moieties, such as, e.g., polyethylene glycol (PEG), etc. Fusion to an antibody Fc region (or a fragment thereof), conjugation to PEG, etc. may find use, e.g., for increasing serum half-life of the antibody upon administration to the subject.

In certain embodiments, when the sialic acid modulator is a sialic acid binding agent, the sialic acid binding agent specifically binds disialyl-T. In one non-limiting example, the sialic acid binding agent is an antibody that specifically binds disialyl-T. As demonstrated in the Experimental section herein, dysregulated Myc promotes the synthesis of disialyl-T on the surface of cancer cells where disialyl-T display promotes tumor growth, e.g., by acting as a Siglec-7 ligand and in turn reducing the immunogenicity of the cancer cells. Accordingly, in some embodiments, the sialic acid modulator is a sialic acid blocking agent, where by “sialic acid blocking agent” is meant an agent (e.g., an antibody) that specifically binds a sialic acid (e.g., disialyl-T) and prevents the sialic acid from serving as a ligand to a receptor (e.g., an immune effector cell receptor, e.g., a Siglec such as Siglec-7. In certain embodiments, the sialic acid modulator is an antibody that specifically binds disialyl-T on the surface of a cancer cell and inhibits or blocks the interaction between disialyl-T and a Siglec (e.g., Siglec-7), e.g., to enhance the immunogenicity of the cancer cell. In certain embodiments, the antibody that specifically binds disialyl-T on the surface of a cancer cell is the anti-disialyl-T antibody QSH2 (see Saito et al. (1994) J Biol Chem. 269(8):5644-52) or an antibody having the binding properties of antibody QSH2. For example, the agent may be an antibody (e.g., a humanized antibody) that competes for binding to disialyl-T with antibody QSH2. Whether a first antibody “competes with” a second antibody for binding to the antigen of interest may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is then added. One of the two antibodies is labeled. If the labeled antibody and the unlabeled antibody bind to separate and discrete sites on the antigen of interest, the labeled antibody will bind to the same level whether or not the suspected competing antibody is present. However, if the sites of interaction are identical or overlapping, the unlabeled antibody will compete, and the amount of labeled antibody bound to the antigen will be lowered. If the unlabeled antibody is present in excess, very little, if any, labeled antibody will bind.

For purposes of the present disclosure, competing antibodies are those that decrease the binding of an antibody to the compound by about 50% or more, about 60% or more, about 70% or more, about 80% or more, about 85% or more, about 90% or more, about 95% or more, or about 99% or more. Details of procedures for carrying out such competition assays are well known in the art and can be found, for example, in Harlow and Lane, Antibodies, A Laboratory Manual, Cold Spring Harbor Laboratory Press, Cold Spring Harbor, N.Y., 1988, 567-569, 1988, ISBN 0-87969-314-2. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the plate may be titrated. The results may be plotted, and the concentrations necessary to achieve the desired degree of binding inhibition may be compared.

Similarly, in certain embodiments, the methods of enhancing immunogenicity of cancers comprising dysregulated Myc comprise administering an effective amount of a sialic acid-binding Ig-like lectin (Siglec) receptor blocking agent to an individual identified as having a cancer comprising dysregulated Myc. Non-limiting examples of Siglec receptor blocking agents that may be employed include agents (e.g., antibodies) that specifically bind a Siglec (e.g., Siglec-7 or Siglec-9) and inhibit or block the interaction between the Siglec and a sialic acid (e.g., disialyl-T) displayed on a cancer cell, e.g., to enhance the immunogenicity of the cancer cell. Non-limiting examples of Siglec receptor blocking agents that may be employed are described in, e.g., U.S. Ser. No. 16/307,428 and WO 2018/006066, the disclosures of which are incorporated herein by reference in their entireties for all purposes. In certain embodiments, the Siglec receptor blocking agent is the anti-Siglec-7 antibody S7.7 (see Hudak et al. (2014) Nat Chem Biol. 10(1): 69-75) or an antibody having the binding properties of antibody S7.7. For example, the agent may be an antibody (e.g., a humanized antibody) that competes for binding to Siglec-7 with antibody S7.7.

Antibodies that specifically bind a sialic acid of interest (e.g., disialyl-T), a Siglec of interest (e.g., Siglec-7), etc. can be prepared using a wide variety of techniques known in the art including the use of hybridoma, recombinant, phage display technologies, or a combination thereof. For example, an antibody may be made and isolated using methods of phage display. Phage display is used for the high-throughput screening of protein interactions. Phages may be utilized to display antigen-binding domains expressed from a repertoire or combinatorial antibody library (e.g., human or murine). Phage expressing an antigen binding domain that binds a sialic acid of interest (e.g., disialyl-T) or a Siglec of interest (e.g., Siglec-7) can be selected or identified with the sialic acid, e.g., using labeled sialic acid bound or captured to a solid surface or bead. Phage used in these methods are typically filamentous phage including fd and M13 binding domains expressed from phage with Fab, Fv (individual Fv region from light or heavy chains) or disulfide stabilized Fv antibody domains recombinantly fused to either the phage gene III or gene VIII protein. Exemplary methods are set forth, for example, in U.S. Pat. No. 5,969,108, Hoogenboom, H. R. and Chames, Immunol. Today 2000, 21:371; Nagy et al. Nat. Med. 2002, 8:801; Huie et al., Proc. Natl. Acad. Sci. USA 2001, 98:2682; Lui et al., J. Mol. Biol. 2002, 315:1063, each of which is incorporated herein by reference. Several publications (e.g., Marks et al., Bio/Technology 1992, 10:779-783) have described the production of high affinity human antibodies by chain shuffling, as well as combinatorial infection and in vivo recombination as a strategy for constructing large phage libraries. In another embodiment, ribosomal display can be used to replace bacteriophage as the display platform (see, e.g., Hanes et al., Nat. Biotechnol. 2000, 18:1287; Wilson et al., Proc. Natl. Acad. Sci. USA 2001, 98:3750; or Irving et al., J. Immunol. Methods 2001, 248:31). Cell surface libraries may be screened for antibodies (Boder et al., Proc. Natl. Acad. Sci. USA 2000, 97:10701; Daugherty et al., J. Immunol. Methods 2000, 243:211). Such procedures provide alternatives to traditional hybridoma techniques for the isolation and subsequent cloning of monoclonal antibodies.

After phage selection, the antibody coding regions from the phage can be isolated and used to generate whole antibodies, including human antibodies, or any desired antigen binding fragment, and expressed in any desired host, including mammalian cells, insect cells, plant cells, yeast, and bacteria. For example, techniques to recombinantly produce Fv, scFv, Fab, F(ab′)₂, and Fab′ fragments may be employed using methods known in the art.

A sialic acid modulator (e.g., an antibody) “specifically binds” to its target (e.g., a sialic acid such as disialyl-T) if it binds to or associates with the target with an affinity or Kₐ (that is, an equilibrium association constant of a particular binding interaction with units of 1/M) of, for example, greater than or equal to about 10⁵ M⁻¹. In certain embodiments, the sialic acid modulator binds to its target with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In certain aspects, specific binding means the sialic acid modulator binds to its target with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the sialic acid modulator for its target can be readily determined using conventional techniques, e.g., by competitive ELISA (enzyme-linked immunosorbent assay), by equilibrium dialysis, by using surface plasmon resonance (SPR) technology (e.g., the BlAcore 2000 instrument, using general procedures outlined by the manufacturer); by radioimmunoassay; or the like.

In certain embodiments, the sialic acid modulator is a small molecule. As used herein, a “small molecule” is a compound having a molecular weight of 1000 atomic mass units (amu) or less. In some embodiments, the small molecule is 750 amu or less, 500 amu or less, 400 amu or less, 300 amu or less, or 200 amu or less. In certain aspects, the small molecule is not made of repeating molecular units such as are present in a polymer. In some embodiments, the sialic acid modulator is a small molecule known to bind to a target of interest (e.g., Myc, St6galnac4, or the like). In other aspects, a suitable small molecule sialic acid modulator is identified, e.g., using a suitable approach for screening small molecules, e.g., by screening a combinatorial library of small molecules.

In certain embodiments, the methods of the present disclosure further include identifying the individual as having a cancer comprising dysregulated Myc. Identifying the individual as having a cancer comprising dysregulated Myc may include, e.g., receiving a report indicating that Myc expression levels are dysregulated in the individual's cancer. According to some embodiments, identifying the individual as having a cancer comprising dysregulated Myc comprises determining that Myc expression levels are dysregulated in the individual's cancer. A variety of approaches may be employed to determine that Myc expression levels are dysregulated in the individual's cancer, including qPCR for Myc RNA obtained from cancer cells of the individual's cancer, quantitative immunoassay for Myc protein obtained from cancer cells of the individual's cancer (e.g., quantitative ELISA, Western blotting, or the like), and any other convenient approach known in the art for determine the expression levels of a gene/protein of interest.

With the benefit of the present disclosure, it will also be appreciated that identifying the individual as having a cancer comprising dysregulated Myc may include assessing the levels of one or more Myc-promoted sialic acids (e.g., disialyl-T) on the surface of cancer cells of the individual and/or assessing the expression levels of one or more Myc-promoted sialic acid biosynthesis pathway components (e.g., a Myc-promoted sialyltransferase, such as St6galnac4) in cancer cells of the individual.

Aspects of the present disclosure also include methods of enhancing immunogenicity of cancers, the methods comprising administering an effective amount of a disialyl-T modulator to an individual identified as having a cancer characterized by cell surface display of disialyl-T. By “disialyl-T modulator” is meant an agent that reduces the abundance of disialyl-T on the surface of cells in the individual, including (or exclusively or substantially exclusively) cancer cells of the individual, as compared to the abundance in the absence of the disialyl-T modulator. The disialyl-T modulator will vary depending upon the selected mechanism for reducing the abundance of disialyl-T on the surface of the cells. In certain embodiments, the disialyl-T modulator is a Myc inhibitor, an inhibitor of disialyl-T biosynthesis, a sialyltransferase inhibitor (e.g., sialyltransferase inhibitor that inhibits a sialyltransferase upregulated by Myc dysregulation, e.g., St6galnac4), an agent that binds to disialyl-T and inhibits or blocks the interaction between disialyl-T and a Siglec receptor (e.g., Siglec-7), and/or the like. Details regarding Myc inhibitors, sialyltransferase inhibitors, inhibiting/blocking agents that find use as disialyl-T modulators (as understood with the benefit of the present disclosure) are described hereinabove and incorporated but not reiterated herein for purposes of brevity.

Aspects of the present disclosure also include methods of enhancing immunogenicity of cancers, the methods comprising administering an effective amount of an agent to an individual identified as having a cancer comprising cell surface expression of a glycoprotein comprising an O-glycosylated mucin-like domain, wherein the agent modulates the glycoprotein. Such methods are based in part on the inventors' findings presented in the Experimental section herein that Siglec-7 binding specificity to disialyl-T glycans is mediated by presentation of disialyl-T ligands in the context of densely O-glycosylated mucin-like domains. For example, it was found that Siglec-7 binds particularly strongly to the CD43 mucin glycoprotein, which is highly expressed on a range of leukemias, including T-ALL. CD43 was identified as a significant Siglec-7 ligand, and knockout of CD43 in cell lines expressing the protein reduces Siglec-7-Fc staining intensity significantly, and biochemical purification of Siglec-7-Fc binding glycoproteins from cell lysates shows CD43 as the major binding partner of Siglec-7. Knockout or blockade of CD43 with an antibody enhanced killing of tumor cell lines by cytotoxic immune cells.

Accordingly, in the methods involving cancer comprising cell surface expression of a glycoprotein comprising an O-glycosylated mucin-like domain, the agent may be one that binds to the glycoprotein and blocks the interaction between the glycoprotein and a Siglec receptor, e.g., Siglec-7. In certain embodiments, such an agent is an antibody. In some embodiments, the agent employed in the methods inhibits expression of the glycoprotein. In some embodiments, the agent (e.g., a protease) employed in the methods degrades the glycoprotein. A non-limiting example of such an agent is a mucin-degrading agent.

In the methods involving cancer comprising cell surface expression of a glycoprotein comprising an O-glycosylated mucin-like domain, the glycoprotein may comprise disialyl-T. In some embodiments, the glycoprotein is glycosylated CD43 (UniProtKB-P16150 (also known as sialophorin)), e.g., CD43 comprising disialyl-T. According to some embodiments, the agent (e.g., an antibody) binds glycosylated CD43 (e.g., CD43 comprising disialyl-T) and blocks the interaction between glycosylated CD43 and Siglec-7. In one non-limiting example, such an agent is MEM-59 (an anti-CD43 antibody that binds sialylated structures near the CD43 N-terminus) (available from Abcam) or an antibody having the binding properties of antibody MEM-59. For example, the agent may be an antibody (e.g., a humanized antibody) that competes for binding to glycosylated CD43 with the MEM-59 antibody. Also by way of example, such an agent may be antibody L10 (an anti-CD43 antibody that binds sialylated structures near the CD43 N-terminus) (available from Thermofisher) or an antibody having the binding properties of antibody L10. For example, the agent may be an antibody (e.g., a humanized antibody) that competes for binding to glycosylated CD43 with the L10 antibody.

Aspects of the present disclosure further include methods of enhancing immunogenicity of a cancer in an individual, where the methods comprise administering an effective amount of a soluble glycosylated CD43 polypeptide to an individual having cancer, where the soluble glycosylated CD43 polypeptide binds to Siglec-7 in the individual. According to some embodiments, the soluble glycosylated CD43 polypeptide comprises disialyl-T. As will be appreciated with the benefit of the present disclosure which demonstrates that glycosylated CD43 (e.g., comprising disialyl-T) is a Siglec-7 ligand to which Siglec-7 binds particularly strongly and where blocking interaction between Siglec-7 and glycosylated CD43 enhances cancer immunogenicity, a soluble glycosylated CD43 polypeptide administered as a therapeutic to an individual having cancer finds use in blocking the interaction between Siglec-7 molecules and cancer cell surface-displayed CD43 glycoproteins, thereby enhancing immunogenicity of the cancer.

By “soluble glycosylated CD43 polypeptide” is meant the glycosylated CD43 polypeptide is not integrated into a cell membrane. In certain embodiments, the soluble glycosylated CD43 polypeptide includes one or more amino acid substitutions that result in the CD43 polypeptide losing its ability to be anchored to a cell membrane, thereby conferring solubility. Alternatively, or additionally, the soluble glycosylated CD43 polypeptide may be a C-terminally truncated form of CD43 where the truncation results in the CD43 polypeptide losing its ability to be anchored to a cell membrane, thereby conferring solubility. As such, according to some embodiments, the soluble glycosylated CD43 polypeptide comprises an N-terminal fragment of CD43. In certain embodiments, the N-terminal fragment comprises amino acids 20-253 of CD43 or a fragment thereof. The amino acid sequence of CD43 (SEQ ID NO:1) is provided in Table 1 below, with amino acids 20-253 underlined.

TABLE 1
CD43 Amino Acid Sequence

| | |
|---|---|
| CD43 | MATLLLLLGVLVVSPDALGSTTAVQTPTSGEPLVSTS |
| (UniProt- | EPLSSKMYTTSITSDPKADSTGDQTSALPPSTSINEG |
| KB - | SPLWTSIGASTGSPLPEPTTYQEVSIKMSSVPQETPH |
| P16150) | ATSHPAVPITANSLGSHTVTGGTITTNSPETSSRTSG |
| (SEQ ID | APVTTAASSLETSRGTSGPPLTMATVSLETSKGTSGP |
| NO: 1) | PVTMATDSLETSTGTTGPPVTMTTGSLEPSSGASGPQ |
| | VSSVKLSTMMSPTTSTNASTVPFRNPDENSRGMLPVA |
| | VLVALLAVIVLVALLLLWRRRQKRRTGALVLSRGGKR |
| | NGVVDAWAGPAQVPEEGAVTVTVGGSGGDKGSGFPDG |
| | EGSSRRPTLTTFFGRRKSRQGSLAMEELKSGSGPSLK |
| | GEEEPLVASEDGAVDAPAPDEPEGGDGAAP |

In addition to optionally including one or more solubility-conferring mutations, a soluble glycosylated CD43 polypeptide of the present disclosure may include one or more mutations that confer one or more other desirable properties upon the polypeptide. Other desirable properties of interest include, but are not limited to, altered (e.g., greater) binding affinity for Siglec-7 relative to a wild-type/native membrane-bound glycosylated CD43 polypeptide.

According to the methods of the present disclosure, the agent (e.g., sialic acid modulator, disialyl-T modulator, agent administered to an individual identified as having a cancer comprising cell surface expression of a glycoprotein comprising an O-glycosylated mucin-like domain, a Siglec ligand (e.g., soluble glycosylated CD43 polypeptide)) may be administered to any of a variety of individuals. In certain embodiments, the individual is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). According to some embodiments, the individual is a human. In certain embodiments, the individual is an animal model (e.g., a mouse model, a primate model, or the like) of a cancer.

The agent is administered to the individual in an effective amount. By “effective amount” is meant a dosage sufficient to produce a desired result, e.g., an amount sufficient to effect beneficial or desired therapeutic (including preventative) results, such as a reduction in a symptom of the cancer, as compared to a control. In some embodiments, an effective amount is sufficient to slow the growth of a tumor, reduce the size of a tumor, and/or the like. An effective amount may be administered in one or more administrations.

Dosing is dependent on severity and responsiveness of the disease state to be treated. Optimal dosing schedules can be calculated from measurements of accumulation of the agent in the body of the individual. The administering physician can determine optimum dosages, dosing methodologies and repetition rates. Optimum dosages may vary depending on the relative potency of the agent, and can generally be estimated based on EC₅₀s found to be effective in in vitro and in vivo animal models, etc. In general, dosage is from 0.01 μg to 100 g per kg of body weight, and may be given once or more daily, weekly, monthly or yearly. The treating physician can estimate repetition rates for dosing based on measured residence times and concentrations of the agent in bodily fluids or tissues. Following successful treatment, it may be desirable to have the subject undergo maintenance therapy to prevent the recurrence of the disease state, where the agent is administered in maintenance doses, once or more daily, to once every several months, once every six months, once every year, or at any other suitable frequency.

The methods of the present disclosure may result in treating the individual's cancer. By treatment is meant at least an amelioration of one or more symptoms associated with the cancer of the individual, where amelioration is used in a broad sense to refer to at least a reduction in the magnitude of a parameter, e.g. symptom, associated with the cancer being treated. As such, treatment also includes situations where the cancer, or at least one or more symptoms associated therewith, are completely inhibited, e.g., prevented from happening, or stopped, e.g., terminated, such that the individual no longer suffers from the cancer, or at least the symptoms that characterize the cancer. “Treating” the cancer may include slowing cancer cell proliferation, the rate of tumor growth, or the like, in the subject.

The agent may be administered to the individual via any suitable route of administration. In some embodiments, the agent is administered via a route of administration independently selected from oral, parenteral (e.g., by intravenous, intratumoral, intra-arterial, subcutaneous, intramuscular, or epidural injection), topical, and intra-nasal administration. According to some embodiments, the agent is administered orally (e.g., in tablet form, capsule form, liquid form, or the like). According to some embodiments, the agent is administered by intratumoral administration (e.g., intratumoral injection)—that is, by administration (e.g., injection) directly on or into cancerous/tumor tissue of the individual having cancer. Local (e.g., intratumoral) administration of the agent finds use, e.g., where it is desirable to avoid systemic sialic acid modulation in the individual, e.g., to prevent or reduce off-target sialic acid modulation in the individual.

The methods of the present disclosure find use in enhancing immunogenicity of a wide variety of cancers. In some embodiments, the individual has a cancer characterized by the presence of a solid tumor, a semi-solid tumor, a primary tumor, a metastatic tumor, a liquid tumor (e.g., a leukemia or lymphoma), and/or the like. In some embodiments, the individual has a cancer selected from breast cancer, glioblastoma, neuroblastoma, head and neck cancer, gastric cancer, ovarian cancer, skin cancer (e.g., basal cell carcinoma, melanoma, or the like), lung cancer, colorectal cancer, prostate cancer, glioma, bladder cancer, endometrial cancer, kidney cancer, leukemia (e.g., T-cell acute lymphoblastic leukemia (T-ALL), acute myeloid leukemia (AML), etc.), liver cancer (e.g., hepatocellular carcinoma (HCC), such as primary or recurrent HCC), a B-cell malignancy (e.g., non-Hodgkin lymphomas (NHL), chronic lymphocytic leukemia (CLL), follicular lymphoma, mantle cell lymphoma, diffuse large B-cell lymphoma, and the like), pancreatic cancer, thyroid cancer, any combinations thereof, and any sub-types thereof.

Aspects of the present disclosure further include methods of assessing whether a cancer of an individual comprises dysregulated Myc. Such methods include assaying cells of the cancer for the abundance of a Myc-promoted cell surface sialic acid, a Myc-promoted sialic acid biosynthesis component, or both. The methods further include assessing whether the cancer comprises dysregulated Myc based on the abundance of the Myc-promoted cell surface sialic acid, the Myc-promoted sialic acid biosynthesis component, or both. In certain embodiments, the methods include assaying cells of the cancer for the abundance of a Myc-promoted cell surface sialic acid. For example, the methods may include assaying cells of the cancer for the abundance of disialyl-T. When the methods include assaying cells of the cancer for the abundance of disialyl-T, in certain embodiments, the methods further include determining the relative ratio of disialyl-T to sialyl-T on the surface of the cancer cells. According to some embodiments, assessing whether the cancer comprises dysregulated Myc comprises comparing the abundance of the Myc-promoted cell surface sialic acid (e.g., disialyl-T) to a threshold abundance, e.g., an abundance of the Myc-promoted cell surface sialic acid determined to be characteristic of dysregulated Myc, which threshold abundance may vary depending upon the particular Myc-promoted cell surface sialic acid being measured.

In certain embodiments, assessing whether the cancer comprises dysregulated Myc comprises assaying cells of the cancer for the abundance of a Myc-promoted sialic acid biosynthesis component. According to some embodiments, the Myc-promoted sialic acid biosynthesis component is a Myc-promoted sialyltransferase, e.g., St6galnac4. The assaying may be for an mRNA that encodes the sialic acid biosynthesis component, the sialic acid biosynthesis component itself (e.g., sialyltransferase protein), and/or the like.

According to some embodiments, assessing whether the cancer comprises dysregulated Myc comprises comparing the abundance of the Myc-promoted sialic acid biosynthesis component (e.g., St6galnac4) to a threshold abundance, e.g., an abundance of the Myc-promoted sialic acid biosynthesis component determined to be characteristic of dysregulated Myc, which threshold abundance may vary depending upon the particular Myc-promoted sialic acid biosynthesis component being measured.

In certain embodiments, the methods of assessing whether a cancer of an individual comprises dysregulated Myc further comprises assaying cells of the cancer for the abundance of one or more Myc target genes. Myc target genes of interest include, e.g., the Myc target genes provided at http://software.broadinstitute.org/gsea/msigdb/cards/HALLMARK_MYC_TARGETS_V1.html.

The methods of assessing whether a cancer of an individual comprises dysregulated Myc may further include determining that the cancer comprises dysregulated Myc based on the assessment. When the cancer is determined to comprise dysregulated Myc, the methods may further include treating the cancer of the individual. According to some embodiments, the treating may include enhancing immunogenicity of the cancer. For example, the treating may include enhancing immunogenicity of the Myc-driven cancer by administering an effective amount of a sialic acid modulator and/or disialyl-T modulator to the individual. The sialic acid modulator may be any of the sialic acid modulators described hereinabove, including but not limited to, any of the Myc inhibitors, inhibitors of sialic acid biosynthesis, sialidases, sialic acid binding agents (e.g., disialyl-T blocking agents, e.g., antibodies), and/or Siglec receptor (e.g., Siglec-7) blocking agents (e.g., antibodies), described hereinabove. According to some embodiments, enhancing immunogenicity of the cancer comprises administering an antibody that specifically binds disialyl-T on the surface of cells of the cancer and inhibits or blocks the interaction between disialyl-T and a Siglec (e.g., Siglec-7). In certain embodiments, enhancing immunogenicity of the cancer comprises administering an antibody that specifically binds a Siglec (e.g., Siglec-7) and inhibits or blocks the interaction between the Siglec and a sialic acid (e.g., disialyl-T) on the surface of cells of the cancer. According to some embodiments, enhancing immunogenicity of the cancer comprises administering an agent (e.g., an antibody) that specifically binds glycosylated CD43 (e.g., CD43 comprising disialyl-T) and inhibits or blocks the interaction between glycosylated CD43 and a Siglec, e.g., Siglec-7.

### Compositions

The present disclosure also provides compositions. In certain embodiments, the compositions find use, e.g., in practicing the methods of the present disclosure.

According to some embodiments, a composition of the present disclosure includes any of the agents described in the Methods section hereinabove, e.g., any of the sialic acid modulators, disialyl-T modulators, and/or agents administered to an individual identified as having a cancer comprising cell surface expression of a glycoprotein comprising an O-glycosylated mucin-like domain, including any combination thereof.

In certain embodiments, a composition of the present disclosure includes the agent present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Pharmaceutical compositions are also provided. The pharmaceutical compositions of the present disclosure include the agent (e.g., any of the sialic acid modulators, disialyl-T modulators, agents administered to an individual identified as having a cancer comprising cell surface expression of a glycoprotein comprising an O-glycosylated mucin-like domain, and/or soluble glycosylated CD43 polypeptides, as described elsewhere herein) and a pharmaceutically acceptable carrier. The pharmaceutical compositions include an effective amount of the agent.

According to some embodiments, the pharmaceutical composition includes an agent (e.g., an antibody) that specifically binds disialyl-T and inhibits or blocks the interaction between disialyl-T and a Siglec (e.g., Siglec-7), and a pharmaceutically acceptable carrier. In certain embodiments, the pharmaceutical composition includes an agent (e.g., an antibody) that specifically binds glycosylated CD43 (e.g., CD43 comprising disialyl-T) and inhibits or blocks the interaction between glycosylated CD43 and a Siglec, e.g., Siglec-7, and a pharmaceutically acceptable carrier. According to some embodiments, the pharmaceutical composition includes a soluble Siglec ligand (e.g., a soluble Siglec-7 ligand) which is a soluble glycosylated CD43 polypeptide, e.g., a soluble glycosylated CD43 polypeptide comprising disialyl-T, and a pharmaceutically acceptable carrier.

The agent can be incorporated into a variety of formulations for administration to an individual. More particularly, the agent can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the agent suitable for administration to an individual (e.g., suitable for human administration) are generally sterile and may further be free of detectable pyrogens or other contaminants contraindicated for administration to a patient according to a selected route of administration.

In pharmaceutical dosage forms, the agent can be administered alone or in appropriate association, as well as in combination, with a second pharmaceutically active compound, e.g., a second anti-cancer agent (including but not limited to small molecule anti-cancer agents). The following methods and carriers/excipients are merely examples and are in no way limiting.

For oral preparations, the agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agent can be formulated for parenteral (e.g., intravenous, intratumoral, intra-arterial, intraosseous, intramuscular, intracerebral, intracerebroventricular, intrathecal, subcutaneous, etc.) administration. In certain aspects, the agent is formulated for injection by dissolving, suspending or emulsifying the agent in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the agent may be prepared by mixing the agent having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the agent may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the agent against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes the agent, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Kits

As summarized above, the present disclosure provides kits. The kits find use, e.g., in practicing the methods of the present disclosure. According to some embodiments, a subject kit includes a pharmaceutical composition that includes any of the sialic acid modulators, disialyl-T modulators, agents that bind glycoproteins comprising an O-glycosylated mucin-like domain, as described hereinabove, which descriptions are incorporated but not reiterated herein in detail for purposes of brevity.

In certain embodiments, provided is a kit that comprises a pharmaceutical composition comprising a sialic acid modulator and instructions for administering the pharmaceutical composition to an individual identified as having a cancer comprising dysregulated Myc to enhance immunogenicity of the cancer. The sialic acid modulator may be any suitable sialic acid modulator, including any of the sialic acid modulators described hereinabove.

According to some embodiments, provided is a kit that comprises a pharmaceutical composition comprising a disialyl-T modulator and instructions for administering the a pharmaceutical composition to an individual identified as having a cancer characterized by cell surface display of disialyl-T to enhance immunogenicity of the cancer. The disialyl-T modulator may be any suitable disialyl-T modulator, including any of the disialyl-T modulators described hereinabove. In certain embodiments, the disialyl-T modulator is an agent (e.g., an antibody) that specifically binds disialyl-T and inhibits or blocks interaction between disialyl-T and a Siglec (e.g., Siglec-7).

In certain embodiments, provided is a kit that comprises a pharmaceutical composition comprising an agent that binds to a glycoprotein comprising an O-glycosylated mucin-like domain and inhibits or blocks interaction between the glycoprotein and a Siglec, and instructions for administering the a pharmaceutical composition to an individual identified as having a cancer comprising cell surface expression of a glycoprotein comprising an O-glycosylated mucin-like domain to enhance immunogenicity of the cancer. The agent modulates the glycoprotein. The agent may be any suitable agent, including any of such agents described hereinabove. In certain embodiments, the agent (e.g., an antibody) specifically binds glycosylated CD43 and blocks the interaction between glycosylated CD43 and a Siglec (e.g., Siglec-7).

The subject kits may include a quantity of the pharmaceutical compositions, present in unit dosages, e.g., ampoules, or a multi-dosage format. As such, in certain embodiments, the kits may include one or more (e.g., two or more) unit dosages (e.g., ampoules) of a composition that includes the agent. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular agent employed, the effect to be achieved, and the pharmacodynamics associated with the agent, in the individual. In yet other embodiments, the kits may include a single multi dosage amount of the composition.

Components of the kits may be present in separate containers, or multiple components may be present in a single container. A suitable container includes a single tube (e.g., vial), ampoule, one or more wells of a plate (e.g., a 96-well plate, a 384-well plate, etc.), or the like.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

The following examples are offered by way of illustration and not by way of limitation.

### Experimental

### Example 1

### Identification of MYC-Promoted Changes to the Tumor Cell Glycocalyx

In this example, T-cell acute lymphoblastic leukemia (T-ALL) was used as a model system for studying the possibility of MYC-promoted changes to the tumor cell glycocalyx. The 4188 T-ALL cell line, used in all subsequent murine experiments, was derived from a primary tumor in the EμSRα-tTA/tetO-MYC mouse model, where MYC expression is controlled by a tetracycline response element in a tissue-specific manner¹. This model has been used widely to study MYC's role in malignant transformation^{2,3}.

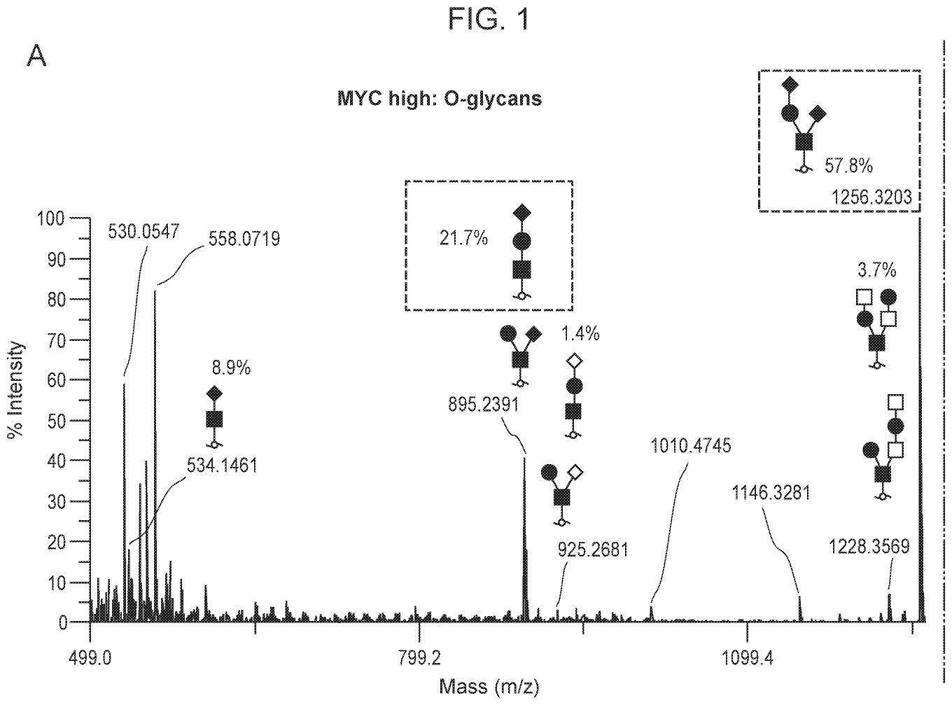

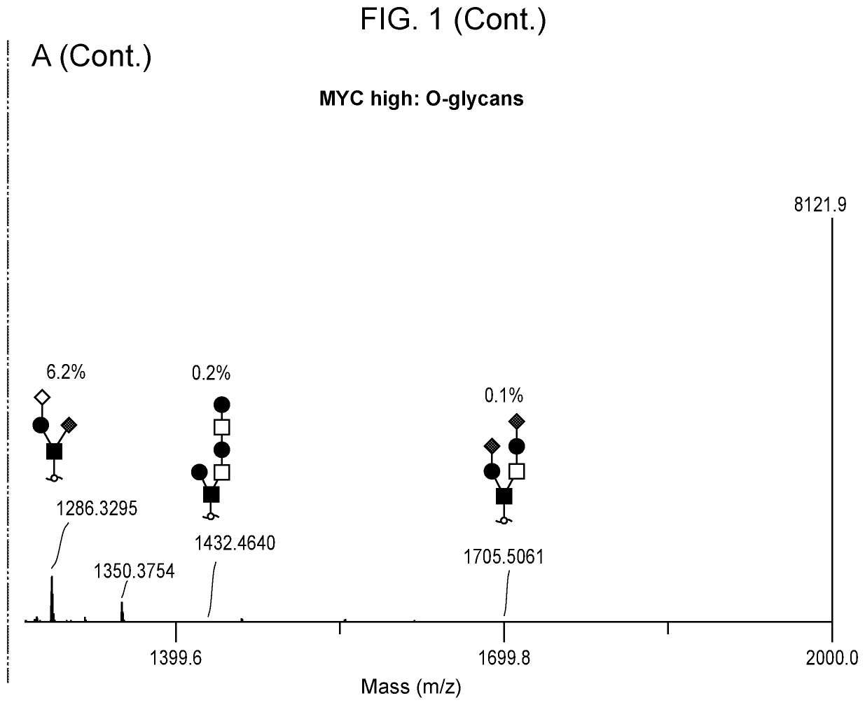

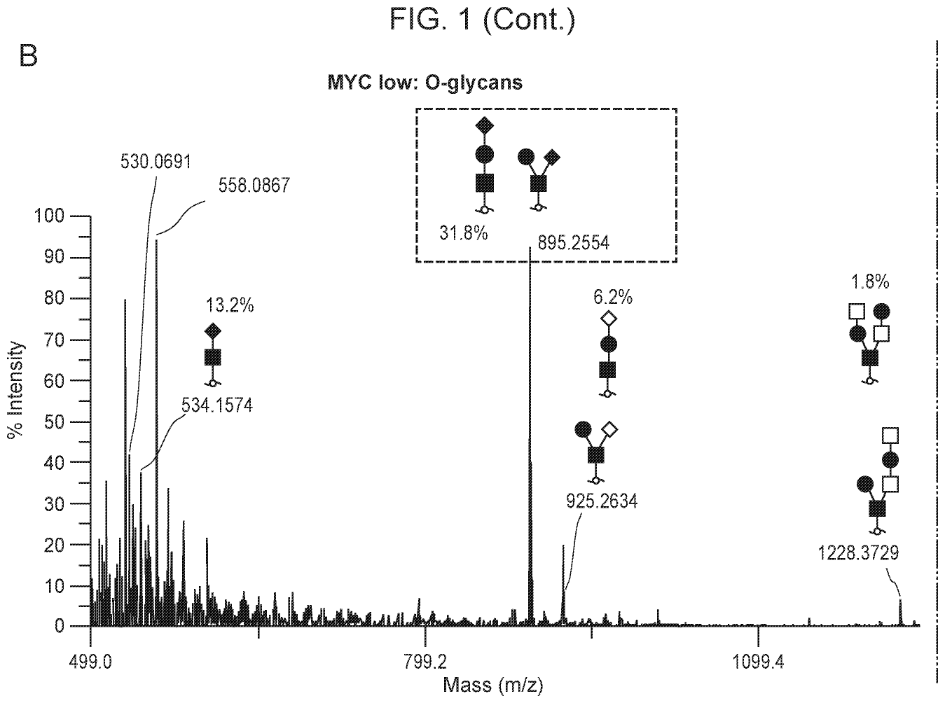

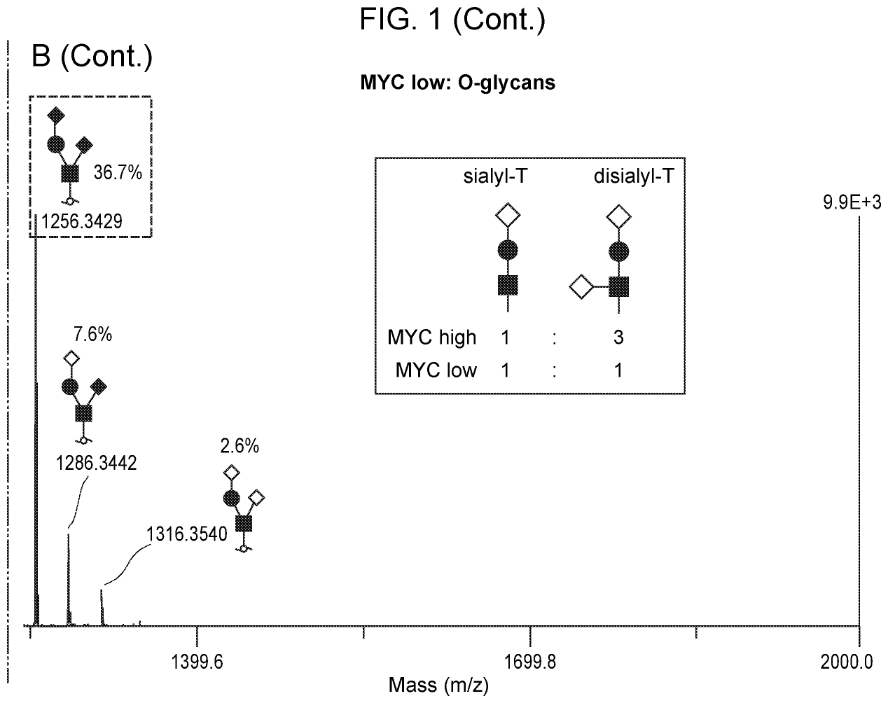

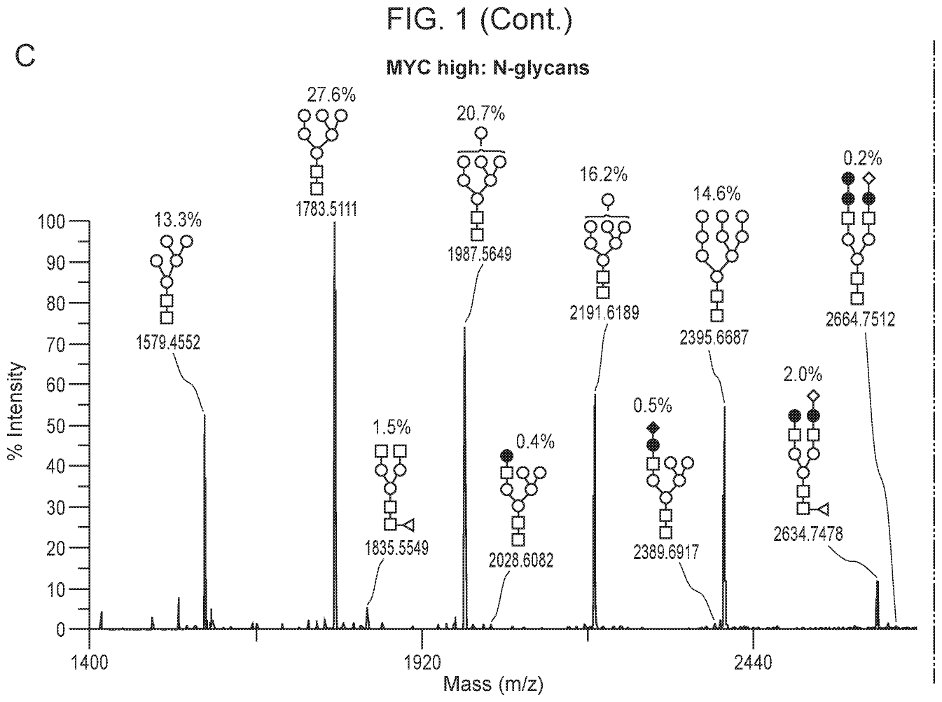

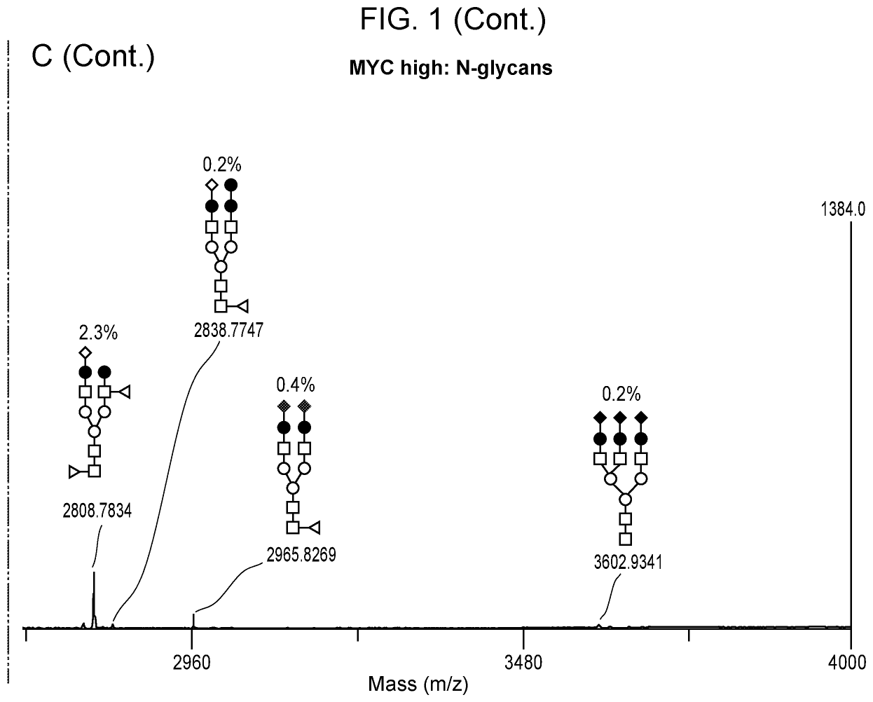

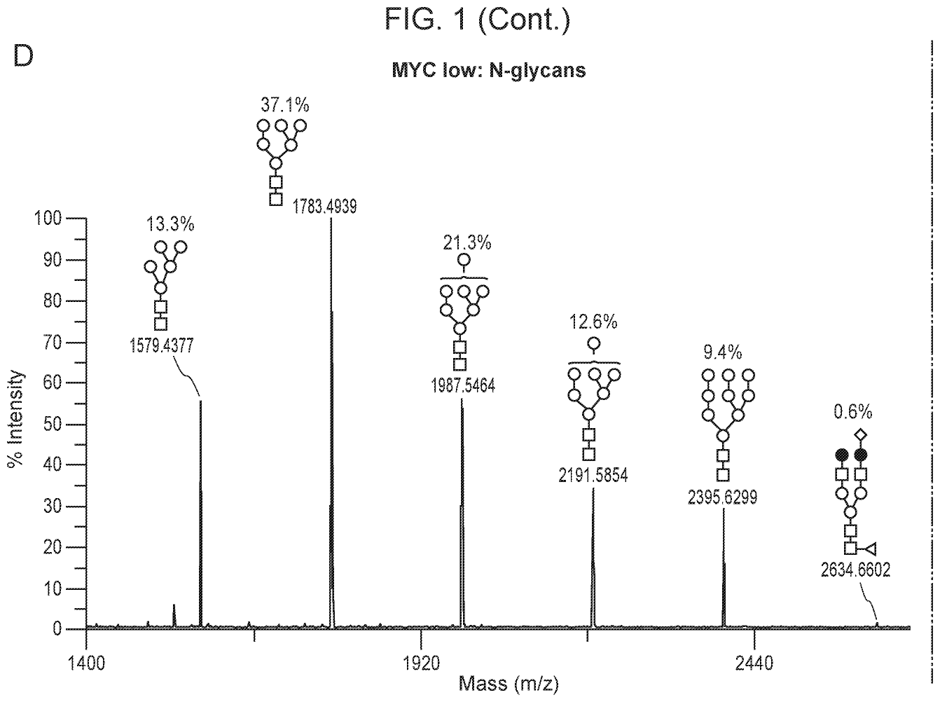

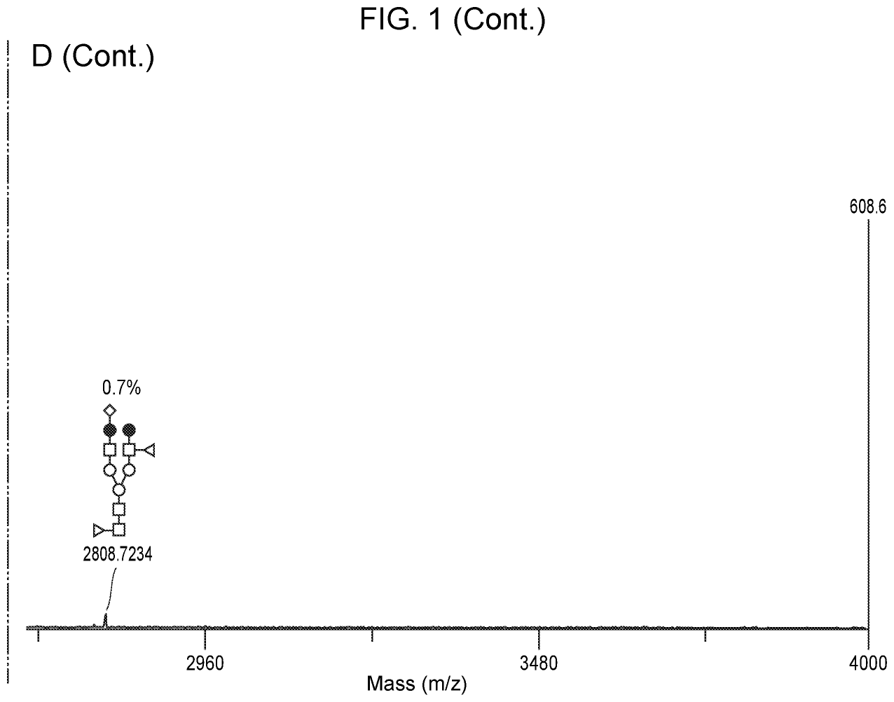

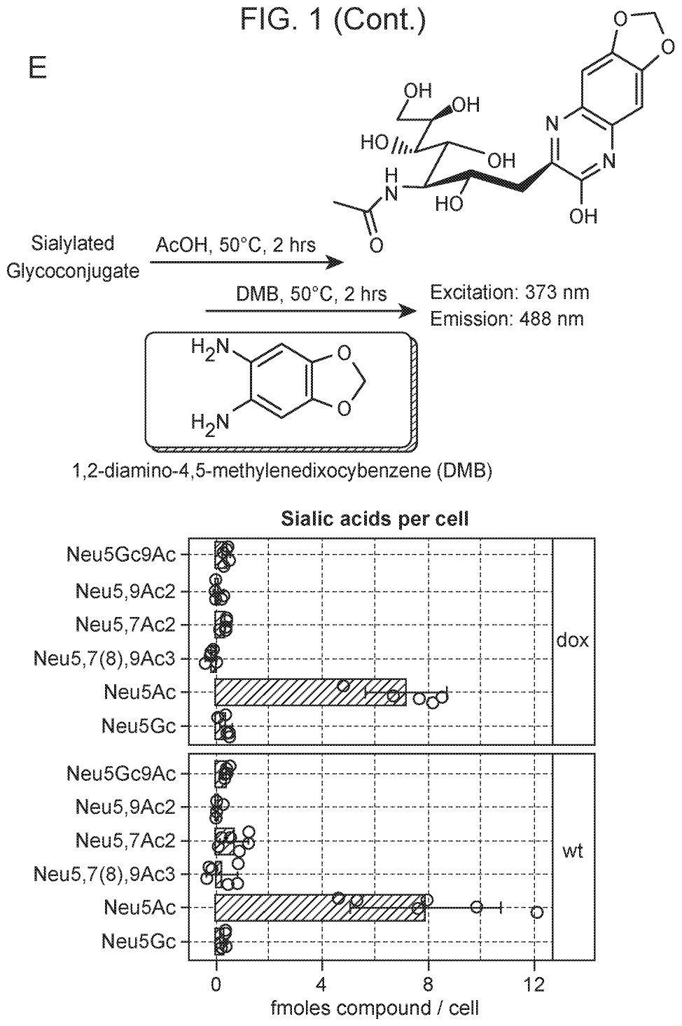

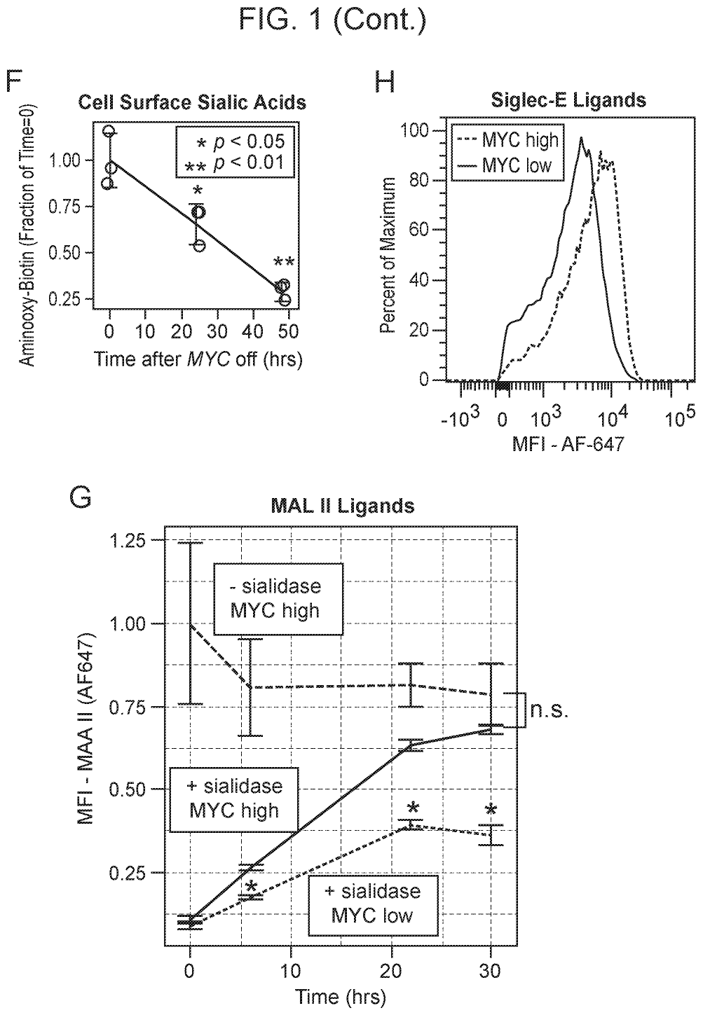

Oncogene-driven changes to the glycocalyx were identified by performing O- and N-glycomics analyses on murine T-ALL cells in the MYC high and low states. Treatment of murine T-ALL with 500 pg/mL doxycycline for 48 hours was performed to shut down MYC transgene expression prior to glycomics. The most prominent O-glycan in the MYC high state was the tetrasaccharide known as disialyl-T, comprising −60% of the total signal. The second most abundant signal was a related trisaccharide, sialyl-T, which comprised only ˜20% of the total signal (FIG. 1, panel A). These glycans are related to one another: both are core 1 one structures, the only difference being the presence of one additional sialic acid in an alpha2-6 linkage to GaINAc in disialyl-T (Neu5Aca2-3Galb1-3[Neu5Aca2-6]GaINAc) relative to sialyl-T (Neu5Aca2-3Galb1-3GaINAc). In contrast, the relative abundance of disialyl-T to sialyl-T fell sharply in the MYC low state to approximately 1:1 (FIG. 1, panel B). Few changes to N-linked glycans were observed in the MYC high versus low states, where high mannose structures and fucosylated glycans were comparably abundant (FIG. 1, panels C and D). These data indicate that MYC promotes display of disialyl-T on murine T-ALL.

MYC could promote display of disialyl-T by either boosting metabolism or more specifically targeting the glycocalyx. Because the primary difference between MYC high and low states lay in the additional sialic acid of disialyl-T compared to sialyl-T, changes to sialic acid metabolism was assessed by measuring concentrations of sialic acid species in whole cell lysate from murine T-ALL. On a per cell basis, there were no differences between the MYC high and low states in terms of sialic acid content (FIG. 1, panel E). However, it was determined that MYC induced targeted changes to sialic acids on the cell surface. By labeling plasma membrane bound sialic acids via periodate aminooxy ligation⁴, a reduction in sialic acid content in the glycocalyx was observed 48 hours after curbing MYC expression (FIG. 1, panel F). Assessed next was whether these changes to the glycocalyx correlated with disialyl-T content. Using staining with the lectin MAL-II as a proxy for disialyl-T, it was determined that MYC supports a program of disialyl-T presentation (FIG. 1, panel G).

In view of these results, one potential function of disialyl-T is to shield cancer cells from the immune system by inhibiting leukocyte activation. The Siglecs are a family of immune receptor tyrosine-based inhibitory motif (ITIM) domain containing proteins expressed by most immune cell subsets that bind to sialylated ligands and transmit inhibitory signals⁵. Therefore, the interaction of cancer disialyl-T with immune cell Siglec may provide a means for evading the immune response. To this end, murine T-ALL in the MYC high and low states were screened against a panel of Siglecs and a specific increase in the sialoside ligands for murine Siglec-E in the MYC high relative to low states was observed (FIG. 1, panel H). Murine Siglec-E, the homolog to human Siglecs-7 and -9, inhibits macrophage function and promotes tumor growth⁶.

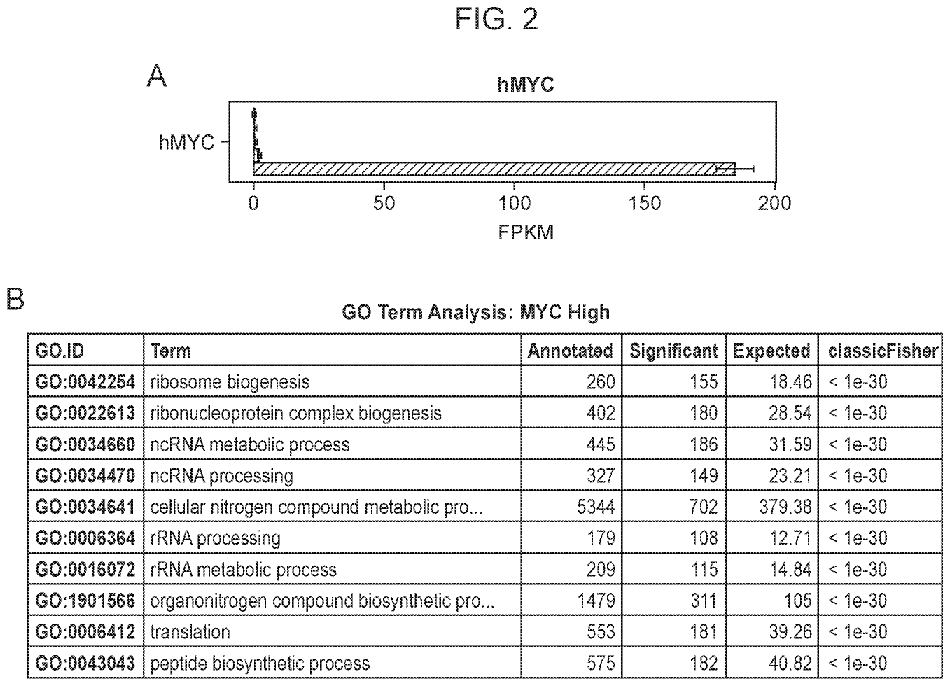

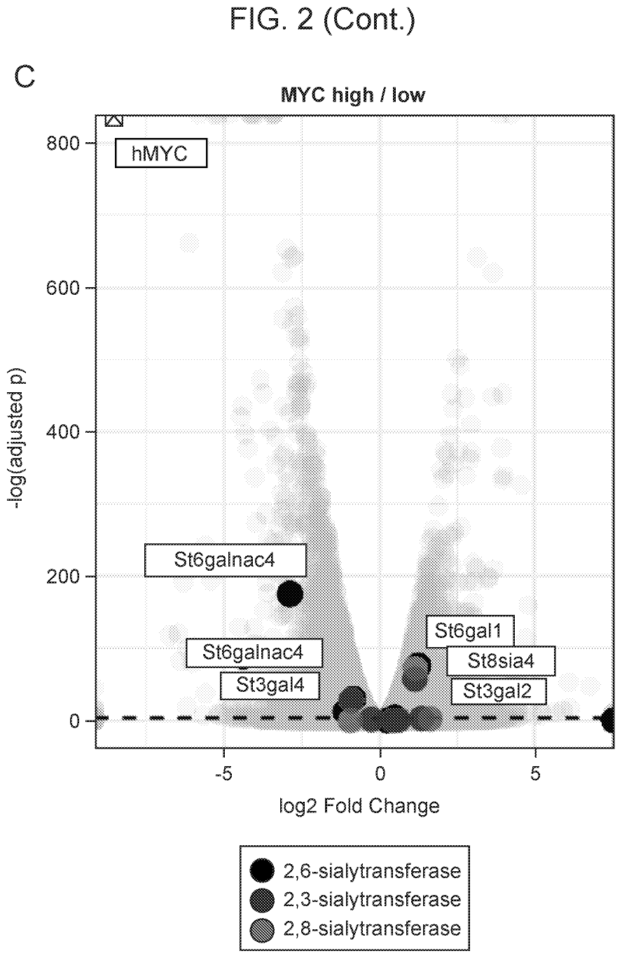

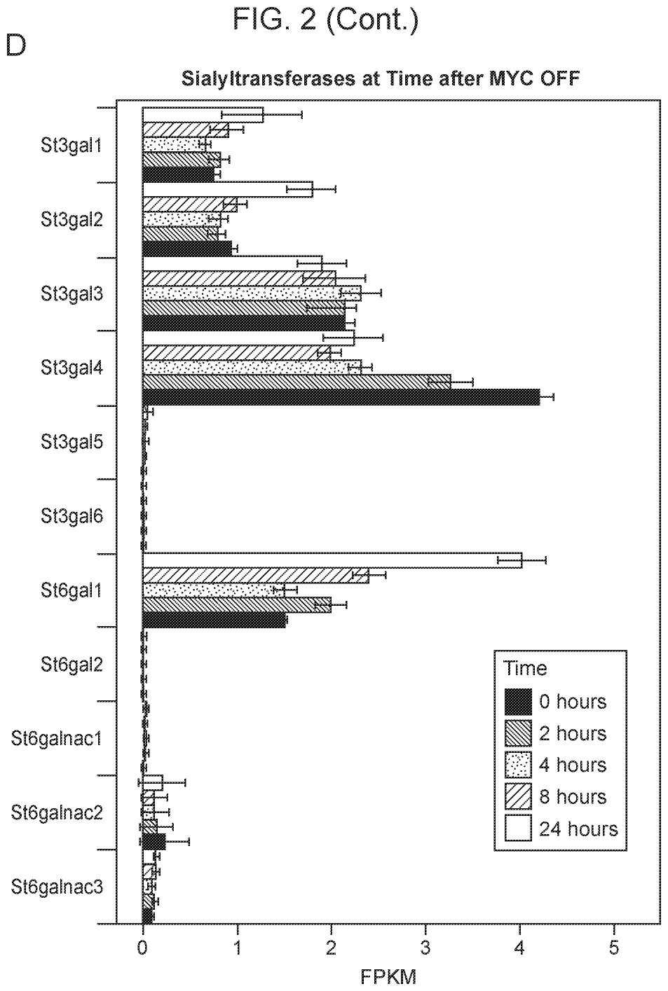

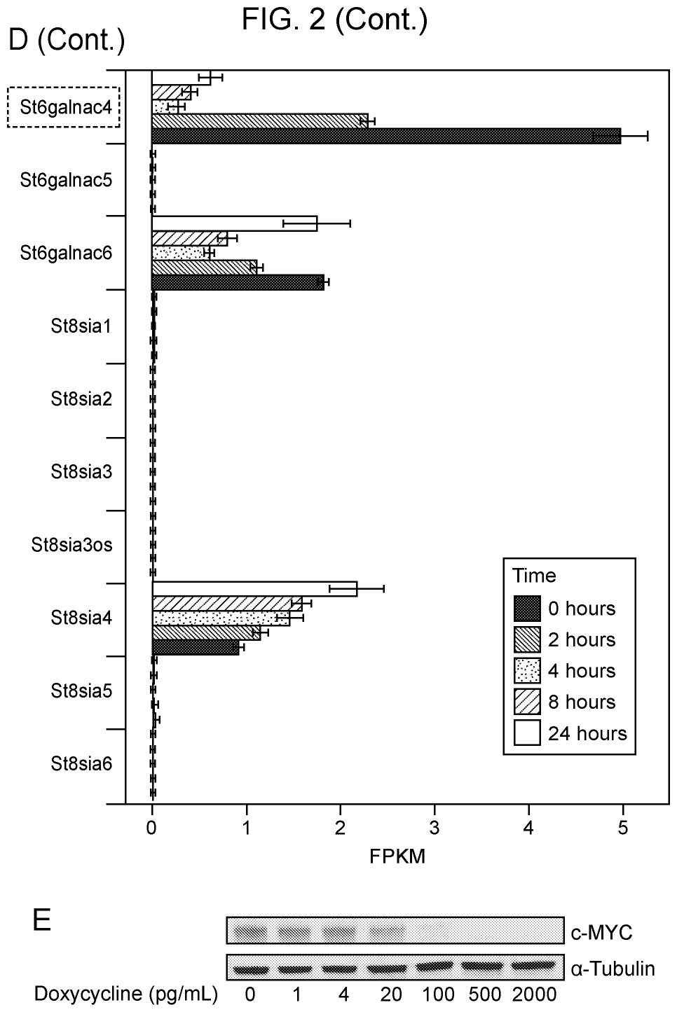

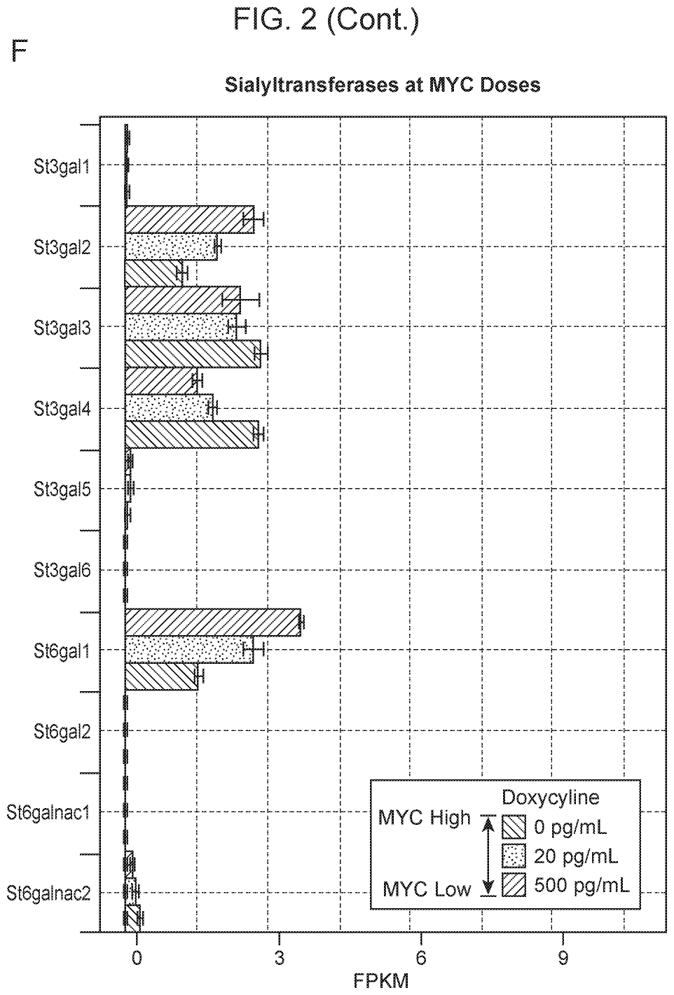

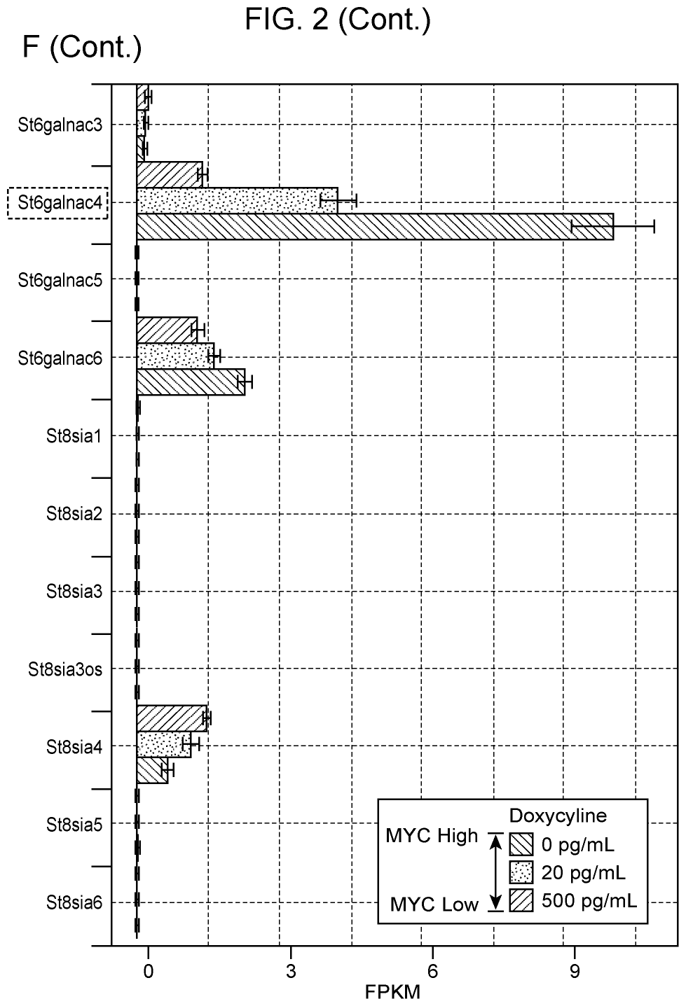

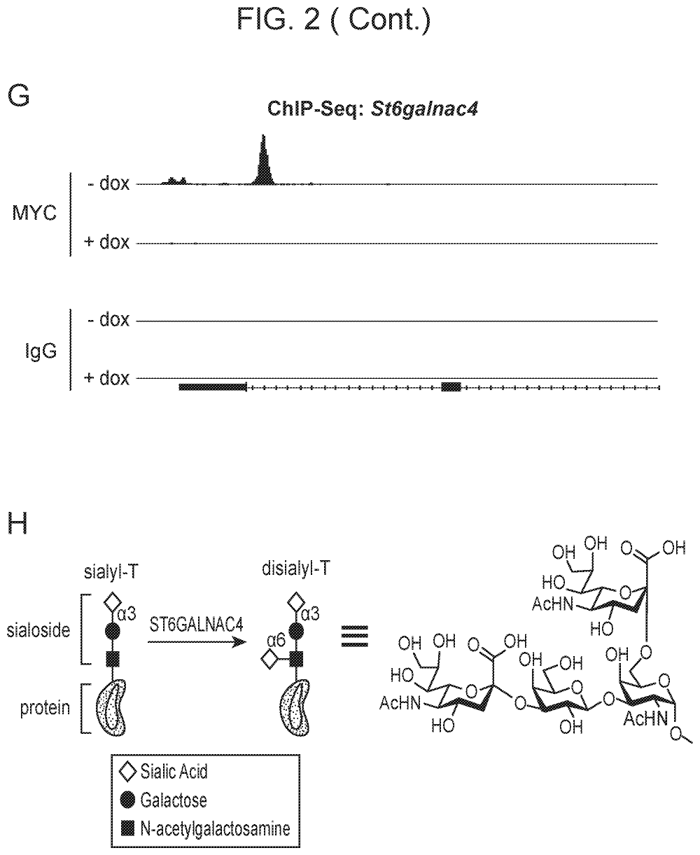

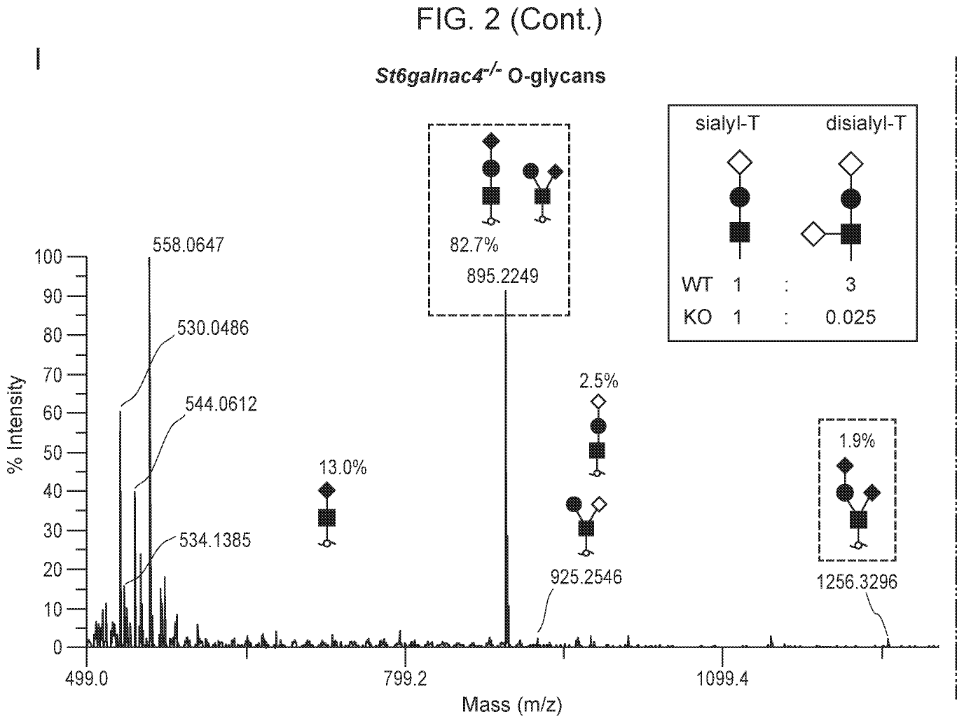

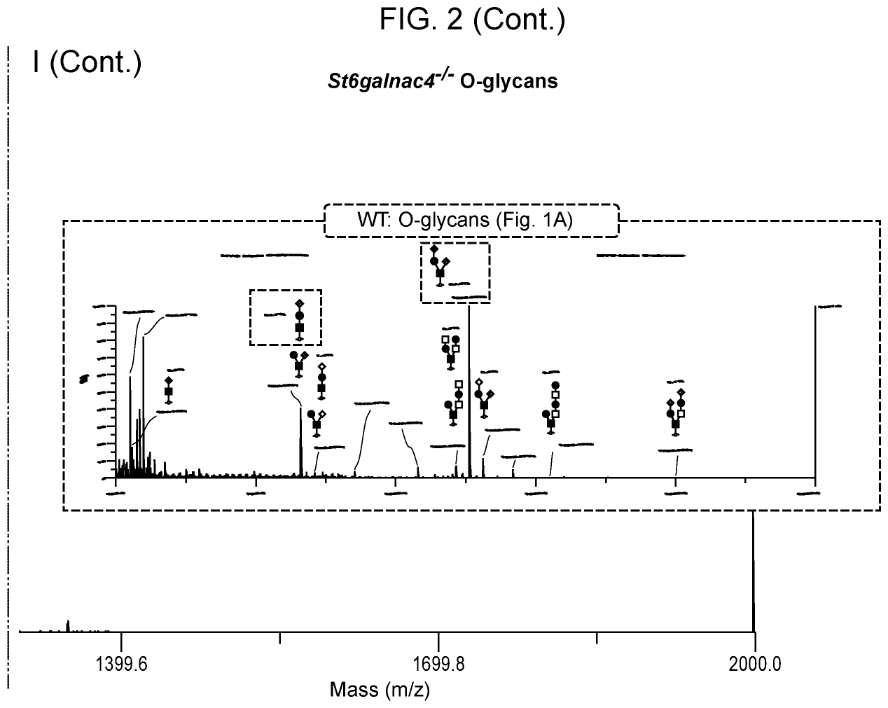

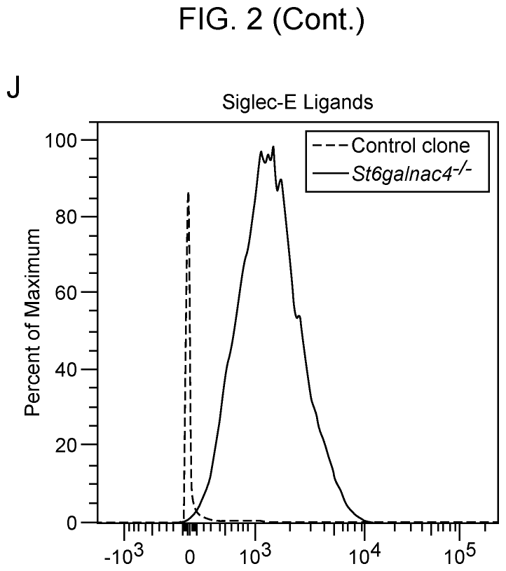

To identify machinery responsible for disialyl-T production in murine T-ALL, RNA-sequencing (RNA-seq) in the MYC high and low states was performed at various timepoints after silencing expression of the MYC transgene. Within 2 hours of doxycycline administration, expression of the MYC transgene was near zero (FIG. 2, panel A). Comparing the MYC high and low states 24 hours after transgene silencing, myriad changes to gene expression consistent with oncogene overexpression was observed (FIG. 2, panel B). The analysis centered on pathways directly relevant to disialyl-T synthesis by focusing on the sialyltransferases, a family of twenty enzymes responsible for attaching sialic acid to nascent glycans. Surprisingly, a single sialyltransferase, St6galnac4, exhibited a pattern of expression highly correlated to MYC (FIG. 2, panel C). St6galnac4 was expressed at elevated levels in the MYC high state, and St6galnac4 expression decreased after short-term MYC silencing (FIG. 2, panel D). It was confirmed that St6galnac4 levels correlated with MYC dose by inducing intermediate MYC levels (FIG. 2, panel E) and an additional RNA-seq experiment was performed (FIG. 2, panel F). Again, St6galnac4 expression was higher than any other sialyltransferase and positively correlated with MYC. ChIP-seq in the same murine T-ALL cell line revealed MYC binding to St6galnac4, suggesting MYC may directly regulate St6galnac4 expression (GSE44672³⁻; FIG. 2, panel G).

Although its physiologic function is poorly characterized, ST6GALNAC4 has been biochemically annotated as a glycoprotein disialyl-T synthase⁷. Specifically, the only reaction ST6GALNAC4 is known to catalyze is the transfer of sialic acid a2-6 to GaINAc in sialyl-T to elaborate disialyl-T glycans (FIG. 2, panel H). Therefore, it was reasoned that MYC promotes disialyl-T display by boosting St6galnac4 expression. To test this hypothesis, St6galnac4−/− cells were generated using CRISPR/Cas9 and O-glycomics was performed. The St6galnac4−/− cells exhibited a near complete depletion of disialyl-T (1.9%) relative to sialyl-T (82.7%). The increase in sialyl-T abundance would also be expected if sialyl-T is the primary substrate for ST6GALNAC4. Comparing the ratio of disialyl-T:sialyl-T on wild type and knockout cells, there is a 120-fold decrease in disialyl-T display in St6galnc4−/− cells relative to control (FIG. 2, panel I). Importantly, the St6galnac4−/− cells exhibited no binding to Siglec-E, indicating that disialyl-T is the primary Siglec-E ligand on murine T-ALL (FIG. 2, panel J).

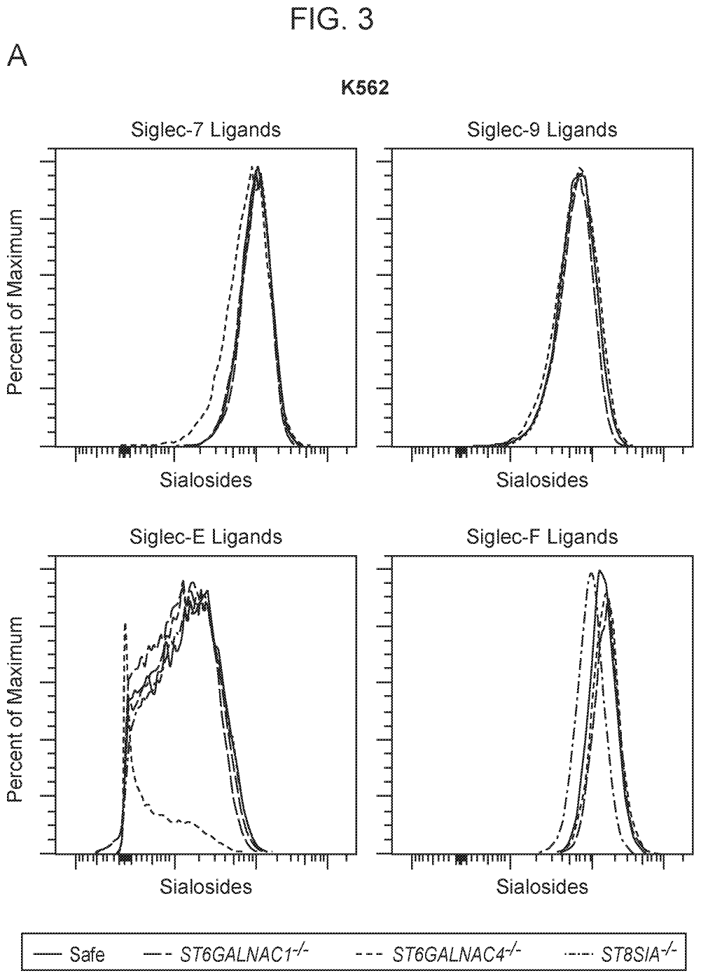

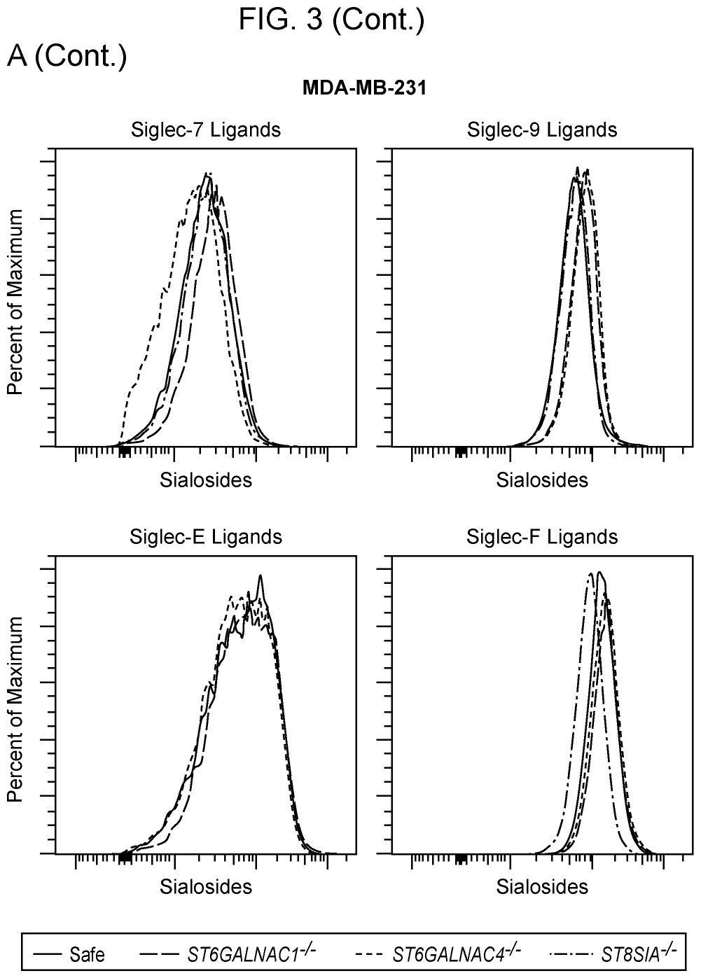

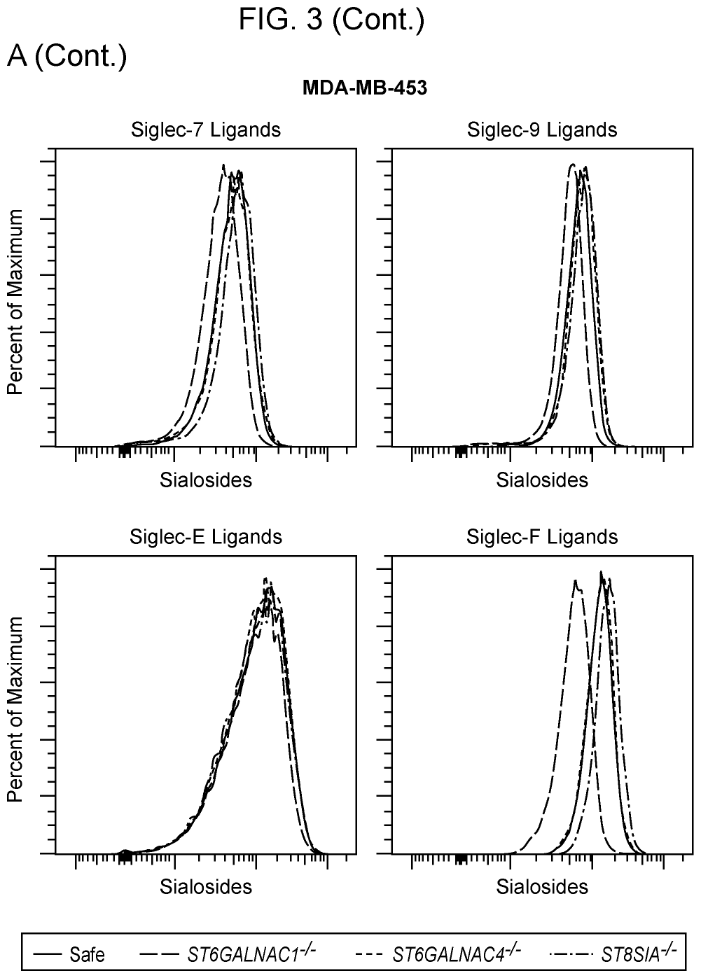

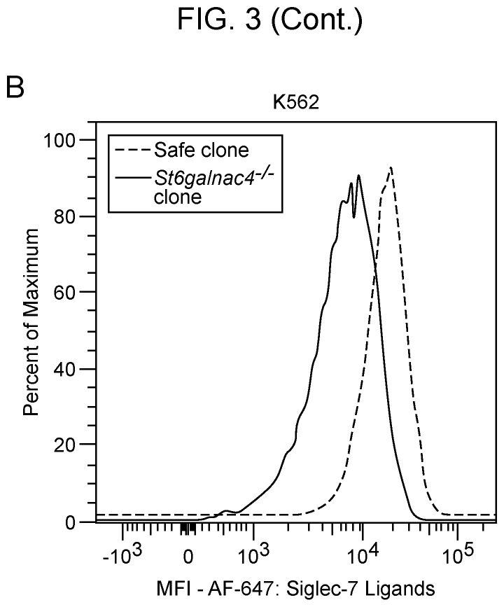

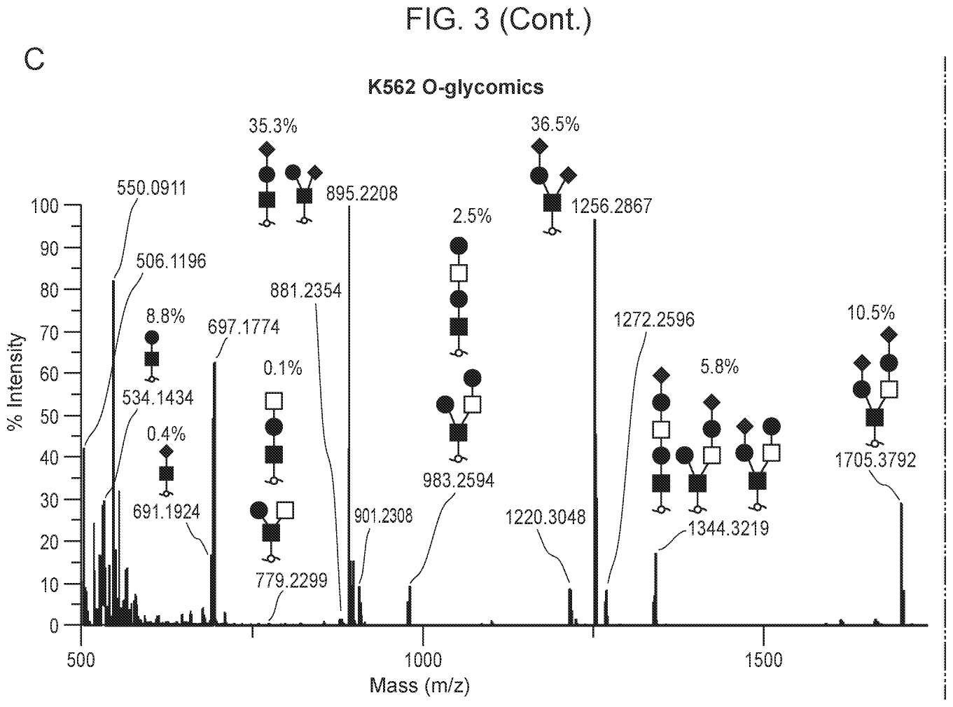

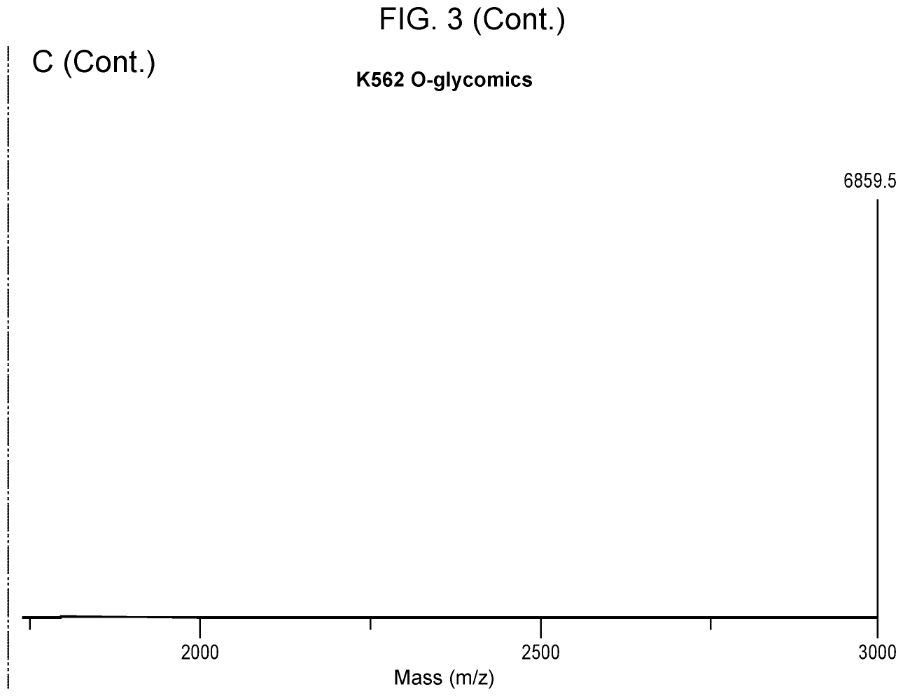

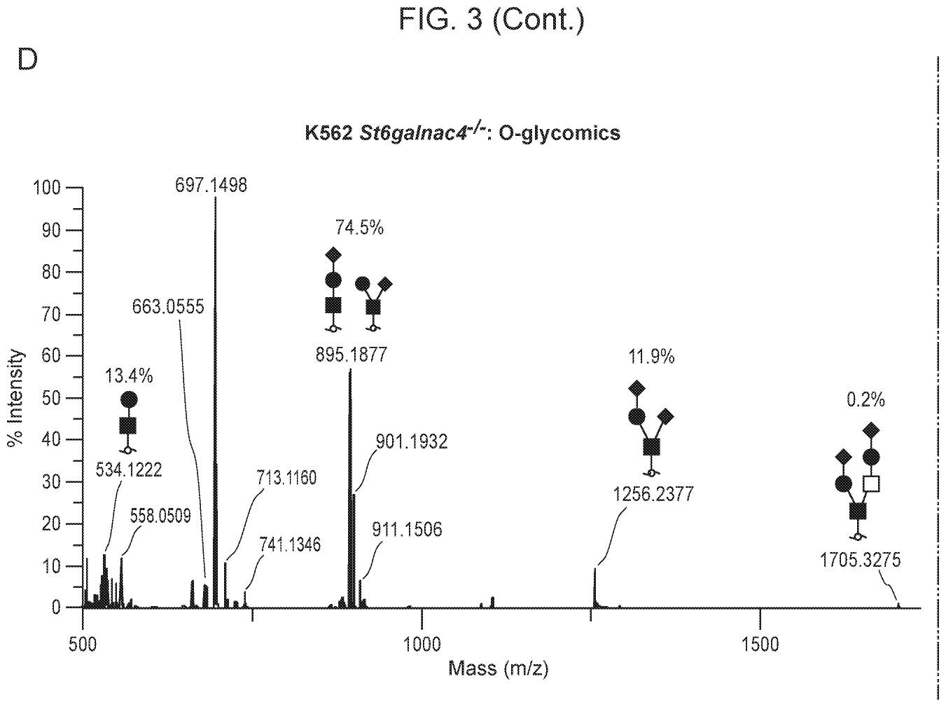

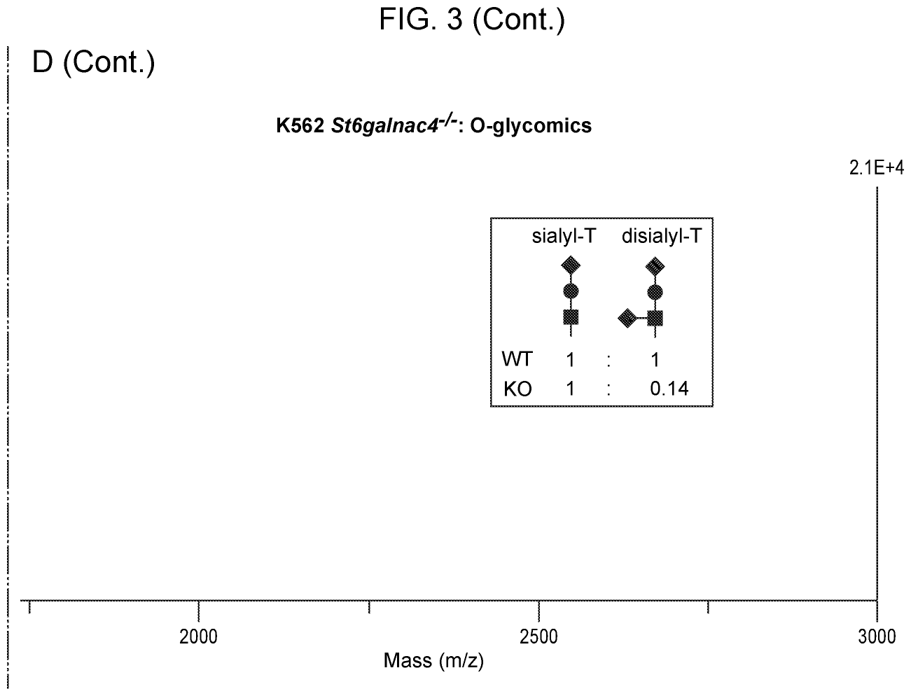

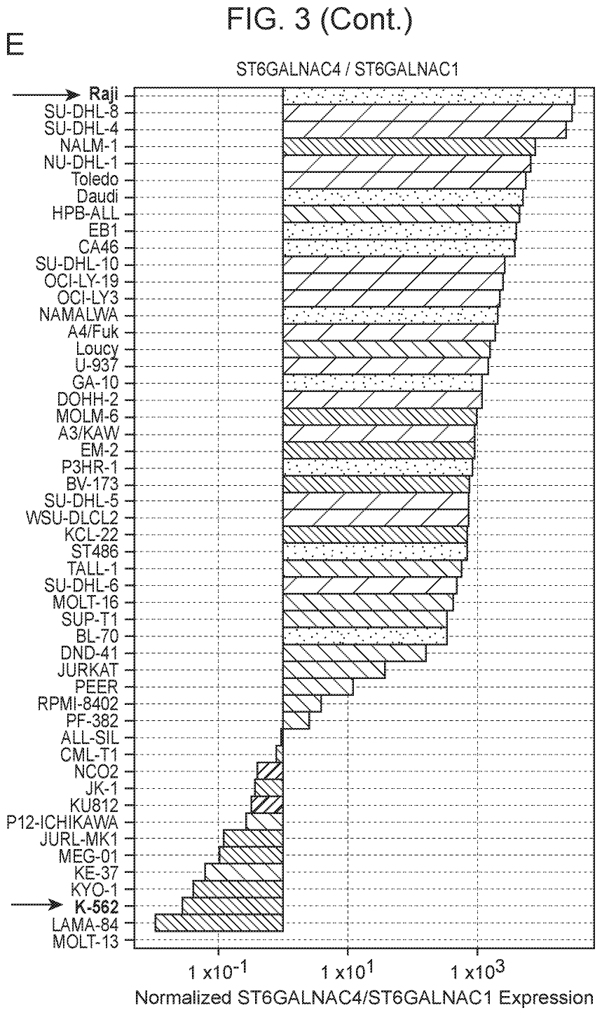

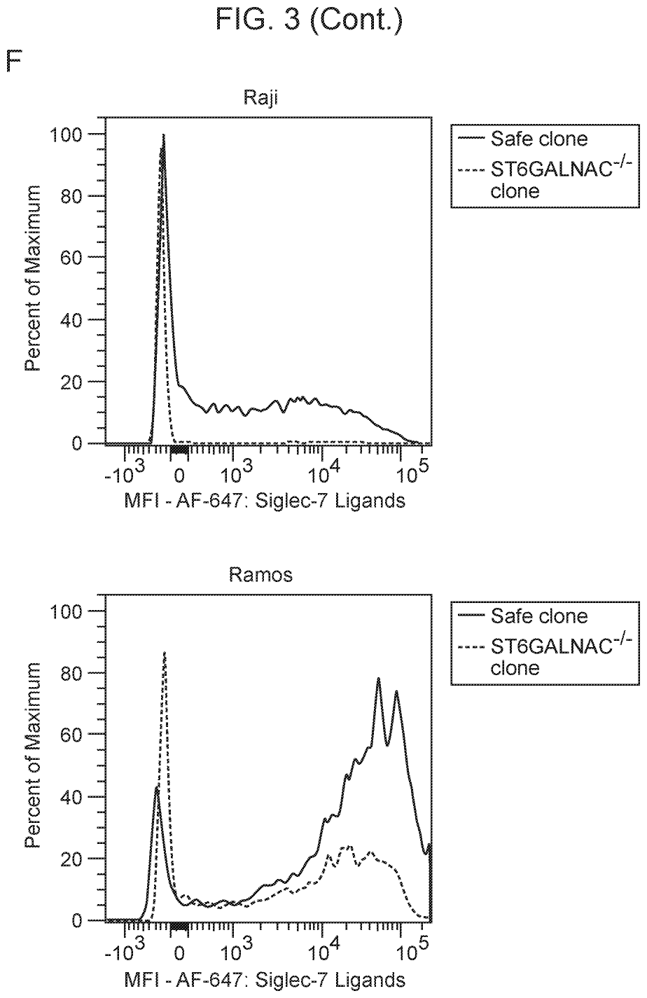

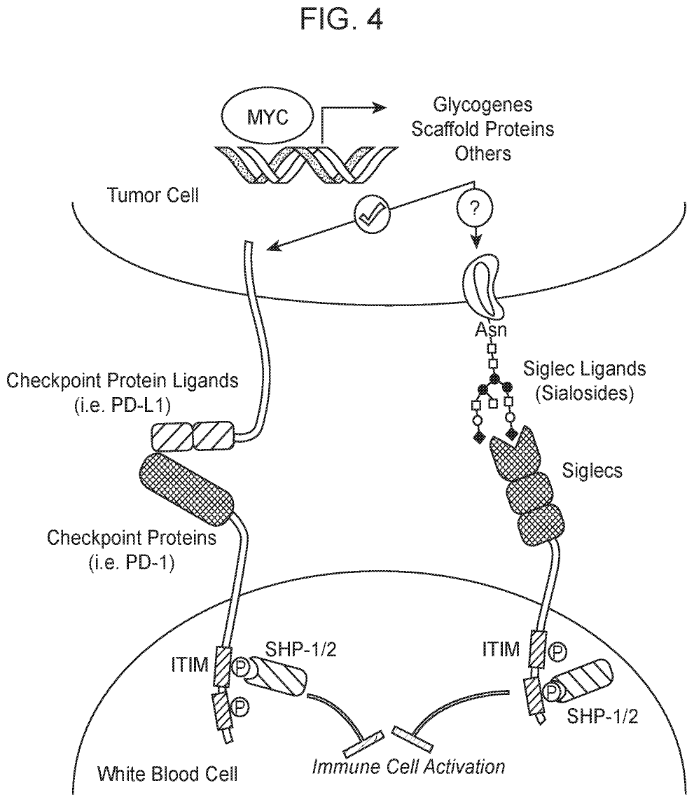

The human homolog of Siglec-E is Siglec-7⁵. To ascertain the contribution of disialyl-T to Siglec-7 binding in human cancer, ST6GALNAC4 and ST6GALNAC1 (a second sialyltransferase biochemically annotated to synthesize disialyl-T from sialyl-T) were knocked out in various human cancer cell lines. In both the breast cancer line MDA-MB-231 and CML line K562, it was found that ST6GALNAC4−/− cells displayed reduced Siglec-7 ligands relative to controls (FIG. 3, panels A and B). Next, it was confirmed that this reduction in Siglec-7 ligands correlated with decreased disialyl-T display on ST6GALNAC4−/− cells by performing O-glycomics (FIG. 3, panels C and D). A meta-analysis of data from the Cancer Cell Line Encyclopedia reveals that K562s have relatively high ST6GALNAC1 expression compared to ST6GALNAC4 (FIG. 3, panel E), implying that MYC controls disialyl-T synthesis in a tissue/cell-type dependent manner. Indeed, the ST6GALNAC4 enriched Burkitt lymphoma line Raji displays a complete loss of Siglec-7 ligands when ST6GALNAC4 is knocked out (FIG. 3, panel F). Therefore, both ST6GALNAC4 and ST6GALNAC1 contribute to synthesis of disialyl-T and the display of Siglec-7 ligands, although MYC regulated ST6GALNAC4 may have an outsized role in tumors from certain lineages.

These data indicate that MYC controls ST6GALNAC4 expression and Siglec-7 ligand display in human leukemias, and that targeting MYC is a means of therapeutically interfering with the cancer cell glycocalyx.

### Materials and Methods

### Row Cytometry

Siglec-Fc reagents (R&D) were pre-complexed at 4 ug/mL with secondary antibodies (Jackson) at 8 ug/mL for 30 min in FACS buffer (0.5% BSA in PBS) on ice. Biotinylated lectins (Vector) at 10 ug/mL were pre-complexed with AlexaFluor-647 labeled streptavidin (Thermo) at 2.5 ug/mL for 30 min in FACS buffer on ice. Cells were washed once with FACS buffer and then incubated with pre-complexed reagents for 30 min on ice. After washing cells twice with FACS buffer, live/dead staining was performed with Sytox Blue (Thermo). Flow cytometry was performed on a BD LSR II.

Sialic Acid Quantification

Sialic acids were quantified in whole cell lysate as previously described via derivatization with 1,2-diamino-4,5-methylenedioxybenzene (Sigma) and detection on an Agilent HPLC. Membrane bound sialic acids were quantified by periodate aminooxy ligation, staining with AlexaFluor-647 labeled streptavidin (Thermo), and subsequent flow cytometry.

### Glycomics

N-glycans were released via PNGase F treatment, and O-glycans were released by reductive beta elimination with sodium borohydride in alkaline conditions. Samples were lyophilized and stored at −20° C. until further use. Samples were subsequently permethylated, extracted with DCM, and analyzed by MALDI-TOF-MS. Additional profiling and fragmentation analysis was performed via Nanospray Ionization—Mass Spectrometry (NSI-MS/FTMS). Permethylated glycans were reconstituted in 50% methanol with 1 mM sodium hydroxide and directly infused into a Thermo Fusion Tribrid Orbitrap. The “TopN” program was used to collect and fragment the top 300 peaks with collision-induced fragmentation (CID).

### RNA-Sequencing

Murine T-ALL was treated with various concentrations of doxycycline for 0 to 48 hours to silence MYC transgene expression. RNA was isolated using the RNeasy kit (Qiagen). Libraries were prepared using a Nextera XT kit (Illumina) and checked for quality with a Bioanalyzer 2100 (Agilent). Paired end 2×150 bp sequencing was performed on a HiSeq 4000 (IIlumina). Sample files were read using bcl2fastq. Reads were mapped using STAR, quantified with featureCounts, and a differential expression analysis was performed with DESeq2.

### Phagocytosis Assays

Bone marrow was harvested from FVB/N mice and stored in liquid nitrogen until needed. Bone marrow cells were cultured in RMPI with 10% FBS and 1% penicillin/streptomycin. Macrophages were differentiated from monocytes by the addition of M-CSF. For phagocytosis assays, murine T-ALL cell were labeled with pHrodo Red (Essen). Phagocytosis events were monitored using the Incucyte Live Cell Imaging System (Essen).

### Mice

All mouse experiments were reviewed and approved by the Stanford University Administrative Panel on Laboratory Animal Care (APLAC). Tumors were seeded by injecting luciferase labeled murine T-ALL cells into the tail vein or subcutaneous tissue. Bioluminesence imaging (BLI) was performed at regular intervals following tumor initiation by administering luciferin and collecting images on an IVIS Spectrum (PerkinElmer).

### MYC Inhibition

MYC transgene expression was silenced in murine T-ALL via administration of 500 pg/mL doxycycline. Human cell lines were treated with 10058-F4 (Sigma) for the indicated time and concentration to inhibit MYC.

### Primary Cell Culture

Primary T-ALL samples were obtained from the Stanford Division of Hematology Tissue Bank. Tumor cells were sorted via flow cytometry as the CD3^{dim} CD7^{high} CD10^{high} CD19^{low} CD11b^{low} CD34^{high} CD123^{low} CD14^{low} population according to preliminary clinical flow cytometry data and cultured on a bed of hTERT stromal cells. Following MYC inhibitor treatment, cells were re-sorted and stained for Siglec ligands.

### REFERENCES

1. Felsher, D. W. & Bishop, J. M. Reversible tumorigenesis by MYC in hematopoietic lineages. Mol. Cell 4, 199-207 (1999).

2. Casey, S. C. et al. MYC regulates the antitumor immune response through CD47 and PD-L1. Science 352, 227-231 (2016).

3. Walz, S. et al. Activation and repression by oncogenic MYC shape tumour-specific gene expression profiles. Nature 511, 483-487 (2014).

4. Zeng, Y., Ramya, T. N. C., Dirksen, A., Dawson, P. E. & Paulson, J. C. High-efficiency labeling of sialylated glycoproteins on living cells. Nature Methods 6, 207-209 (2009).

5. Macauley, M. S., Crocker, P. R. & Paulson, J. C. Siglec-mediated regulation of immune cell function in disease. Nat. Rev. Immunol. 14, 653-666 (2014).

6. Läubli, H. et al. Engagement of myelomonocytic Siglecs by tumor-associated ligands modulates the innate immune response to cancer. Proc. Natl. Acad. Sci. U. S. A. 111, 14211-14216 (2014).

7. Harduin-Lepers, A. et al. Cloning, expression and gene organization of a human Neu5Acα2-3Galβ1-3GaINAc α2,6-sialyltransferase: hST6GaINAc IV. Biochemical Journal 352, 37-48 (2000).

8. Jaiswal, S. et al. CD47 is upregulated on circulating hematopoietic stem cells and leukemia cells to avoid phagocytosis. Cell 138, 271-285 (2009).

9. Hummel, M. et al. A biologic definition of Burkitt's lymphoma from transcriptional and genomic profiling. N. Engl. J. Med. 354, 2419-2430 (2006).

10. Bond, M. R. et al. Metabolism of diazirine-modified N-acetylmannosamine analogues to photo-cross-linking sialosides. Bioconjug. Chem. 22, 1811-1823 (2011).

### Example 2

### Identification of Siglec-7/0043 Interaction and its Suppression of Activation of Cytotoxic Immune Cells

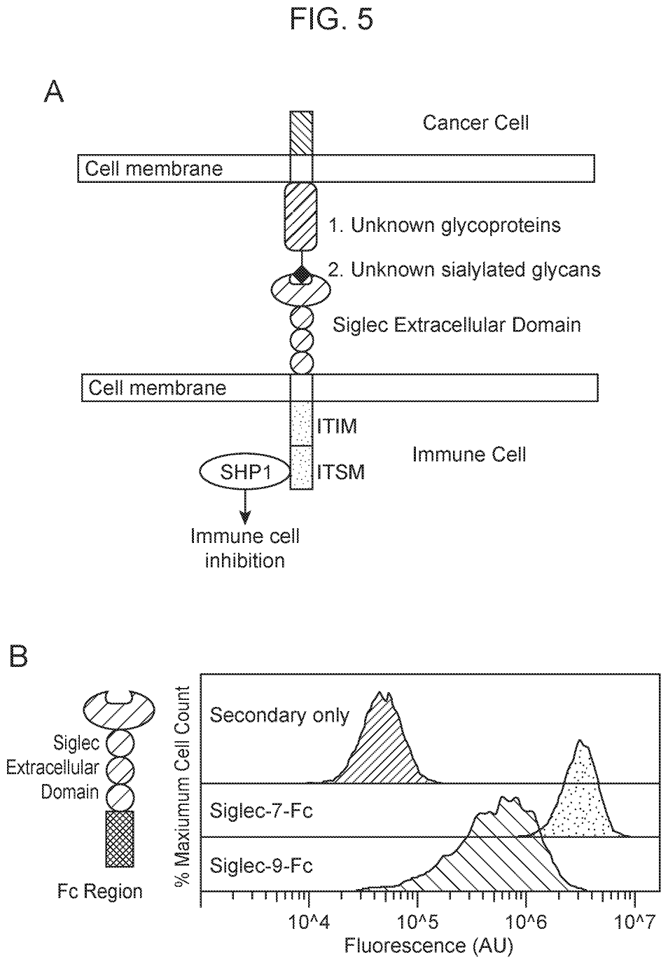

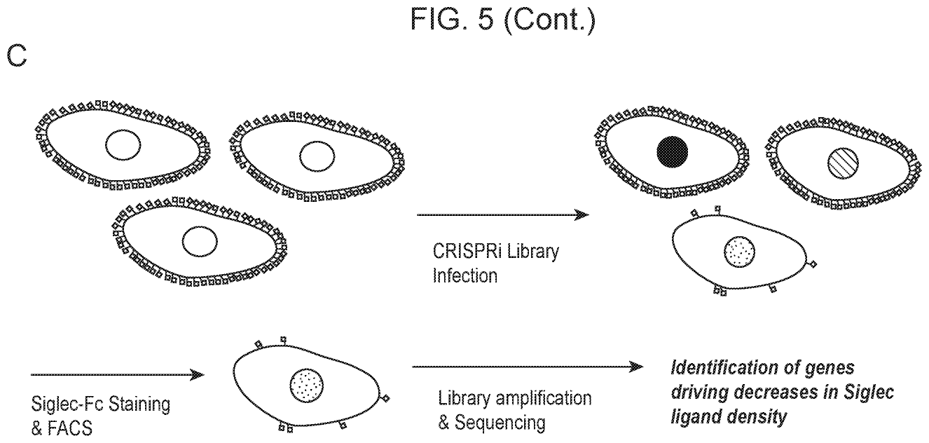

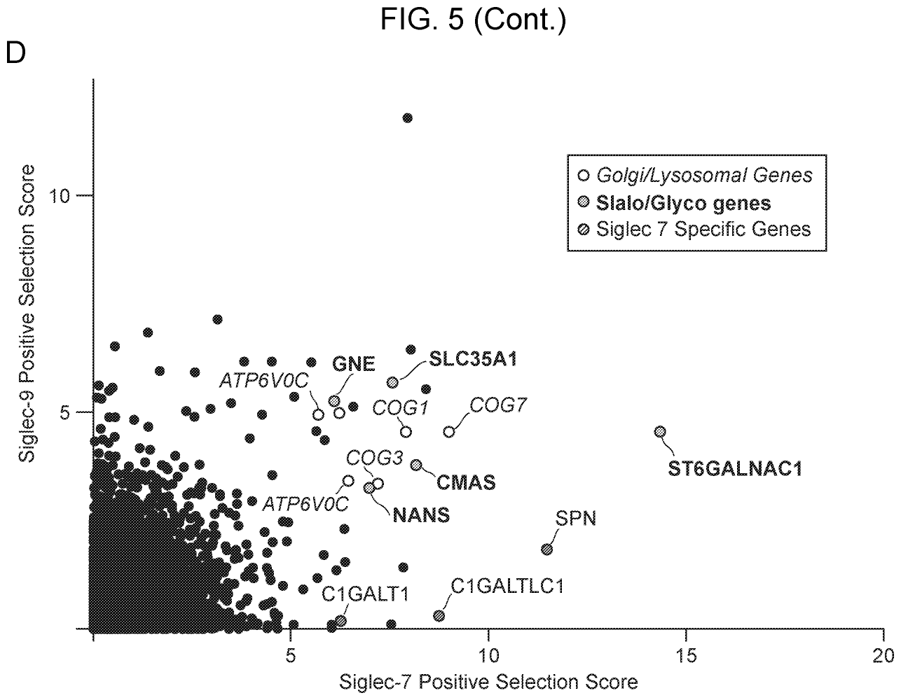

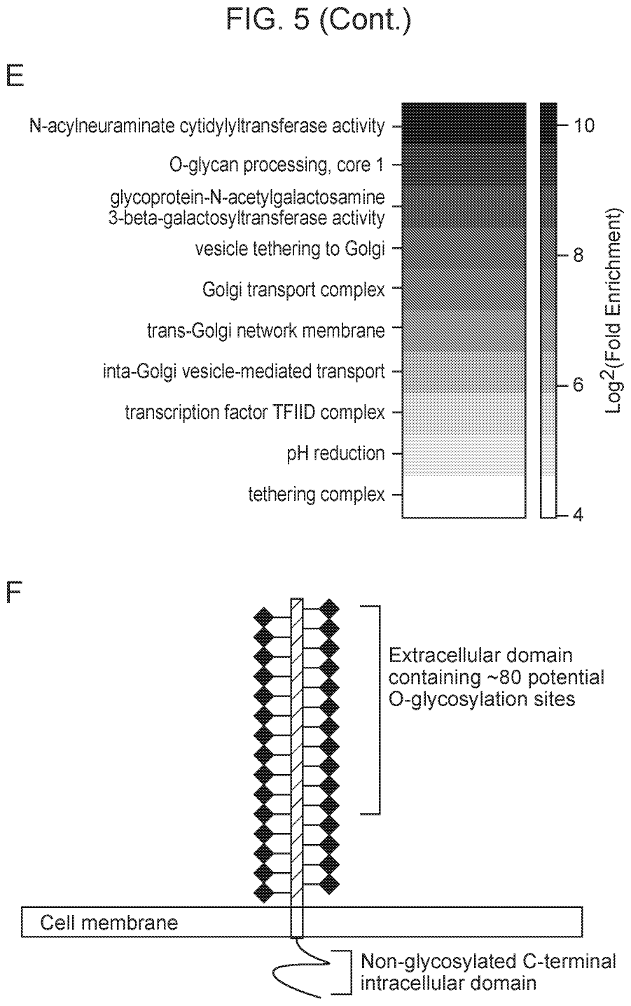

In this example, a genome-wide CRISPRi screen was performed to identify core drivers of cell-surface Siglec ligand expression. Results are shown in FIG. 5. Panel A:

Domain architecture of Siglec glyco-immune checkpoint receptors. Siglec receptors bind to sialylated glycans to inhibit activation of immune cells. Panel B: Cell surface staining of K562 cells with Siglec-7-Fc and Siglec-9-Fc chimeric proteins. Chimeras were precomplexed with Anti-hFc-AlexaFluor488 at 1 ug/ml for 1 hour, staining was performed on ice for 30 minutes and cells were analyzed by flow cytometry. Panel C: Workflow of genome-wide CRISPRi screen to identify genes involved in Siglec ligand biogenesis. 100×106 CRISPRi library-infected K562 dCas9-KRAB cells were stained with Siglec-7-Fc and Siglec-9-Fc proteins. FACS was performed to isolate a population of cells exhibiting a 10fold reduction in cell surface staining. Panel D: Results of Siglec-7 and Siglec-9 CRISPRi screens. Genes ranked by positive selection score, where a higher score indicates a greater tendency for sgRNAs against that gene to be enriched in low Siglec-Fc staining cells. Panel E: GO analysis of screen hits. Analysis was performed using GOrilla to find GO terms enriched with a p value<10⁵. The top 10 terms for component, function and pathway analysis are shown ranked by fold enrichment. Panel F: Domain structure of CD43 (SPN), a top hit obtained in the Siglec-7 but not Siglec-9 genome-wide screen.

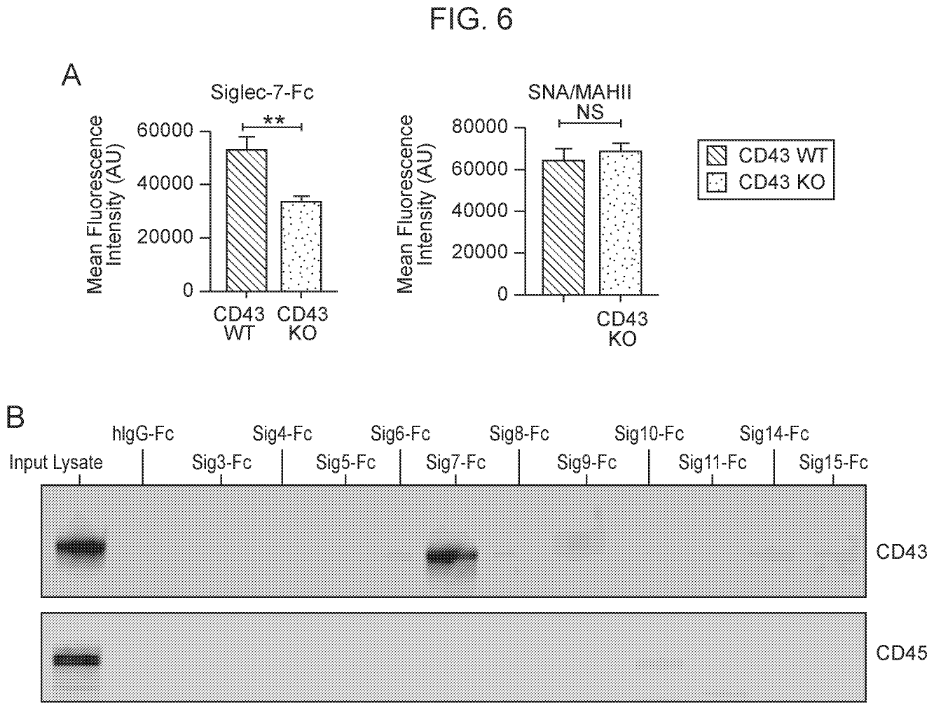

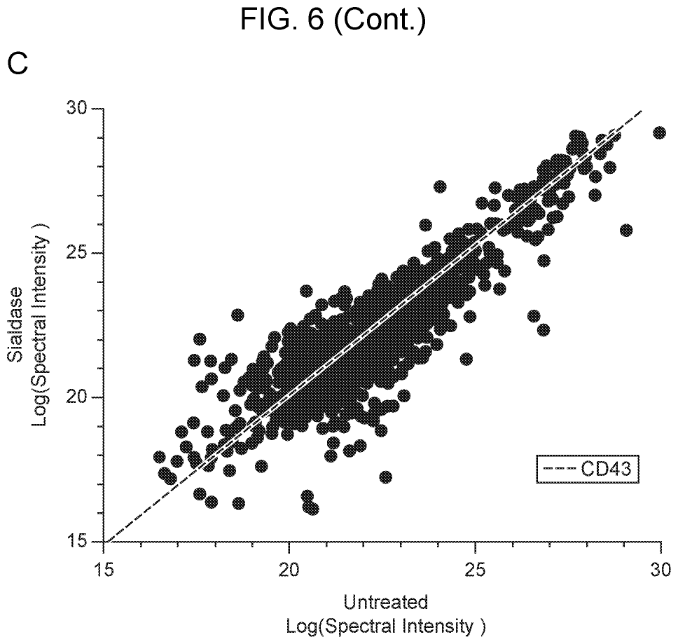

Next, it was determined that CD43 is the major cell-surface glycoprotein ligand for Siglec-7. Results are shown in FIG. 6. Panel A: Labeling intensity of CD43 KO cells generated by CRISPR-Cas9 editing with Siglec-7-Fc and SNA/MAH-11. Siglec-7-Fc labeling is reduced by CD43 knockout, while labeling with the general sialic acid-binding lectins MAH-11 and SNA is not. Mean fluorescence intensity over 3 independent replicates is indicated. Panel B: Enrichment of lysates with recombinant Siglec-Fc constructs. 5×10⁶ K562 cells were lysed in PBS with 0.1% NP-40 and enriched over beads coated with indicated Siglec-Fc chimeras. Interacting proteins were eluted off of the beads and immunoblotting for CD43 was subsequently performed. Panel C: Enrichment of lysates with recombinant Sig7-Fc followed by tryptic digest and MS analysis shows CD43 as the primary enriched cell surface protein. Identified proteins were ranked by combined spectral intensity of all peptides corresponding to that protein ID. Enrichment of proteins from untreated vs sialidase treated lysates was performed to identify proteins binding in a sialic acid-dependent manner. CD43 was identified at high intensity in Siglec-7-Fc pulldowns from untreated lysates but not from sialidase treated lysates.

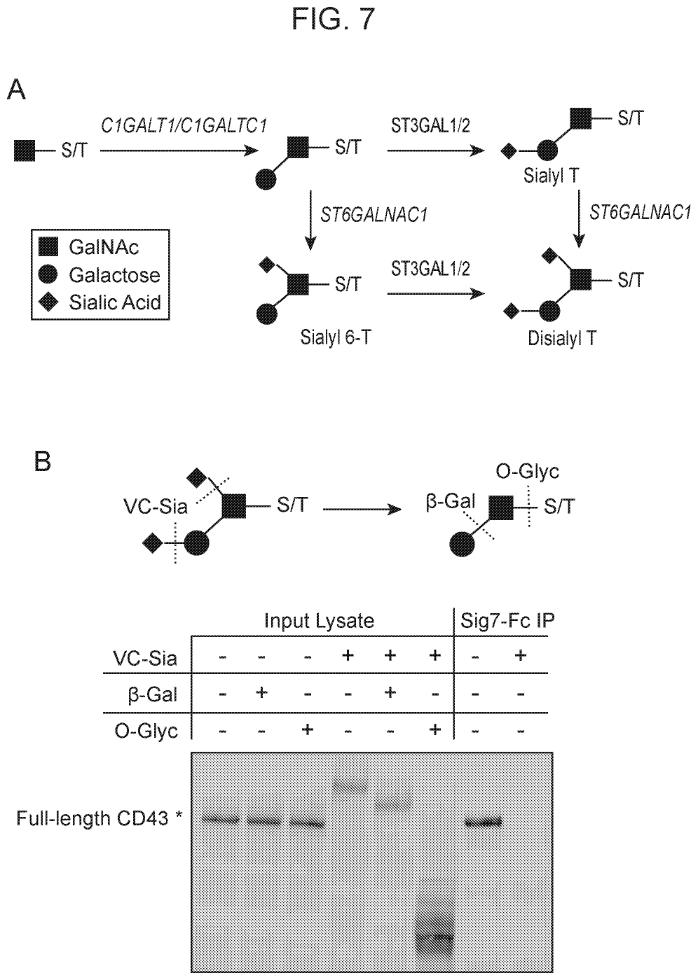

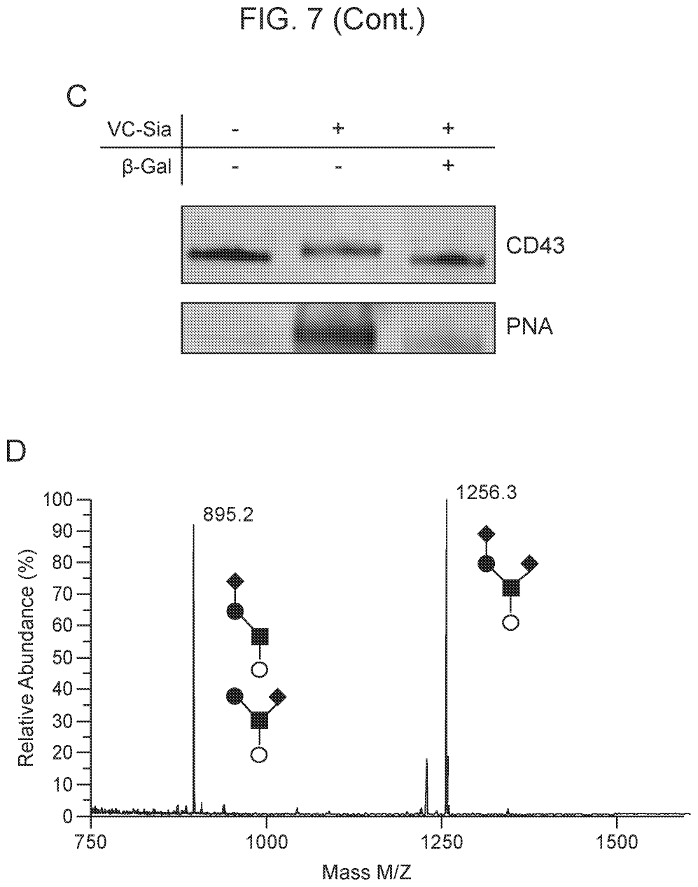

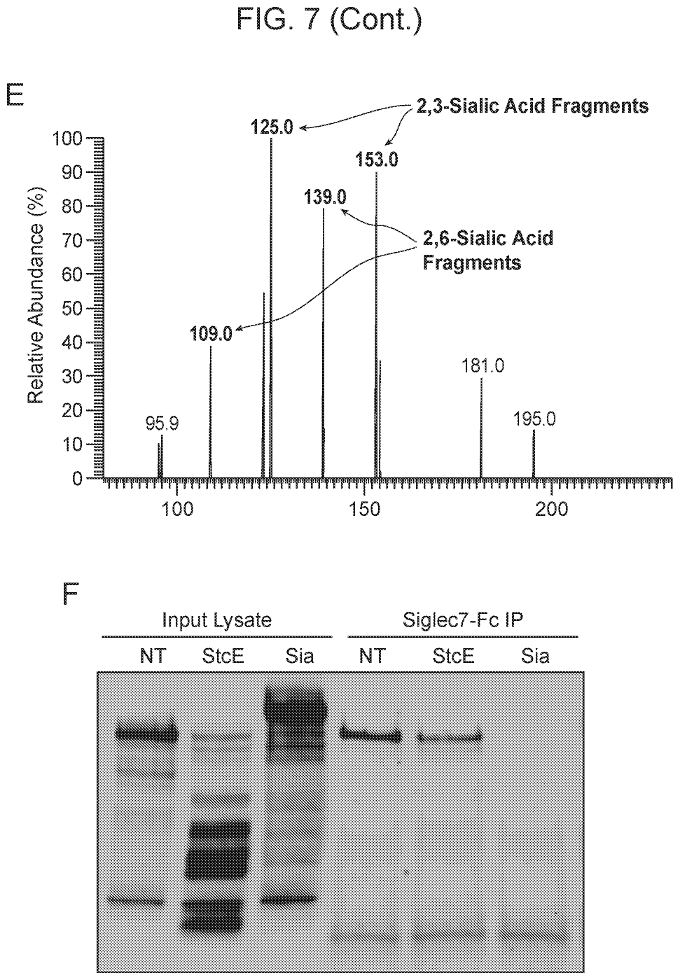

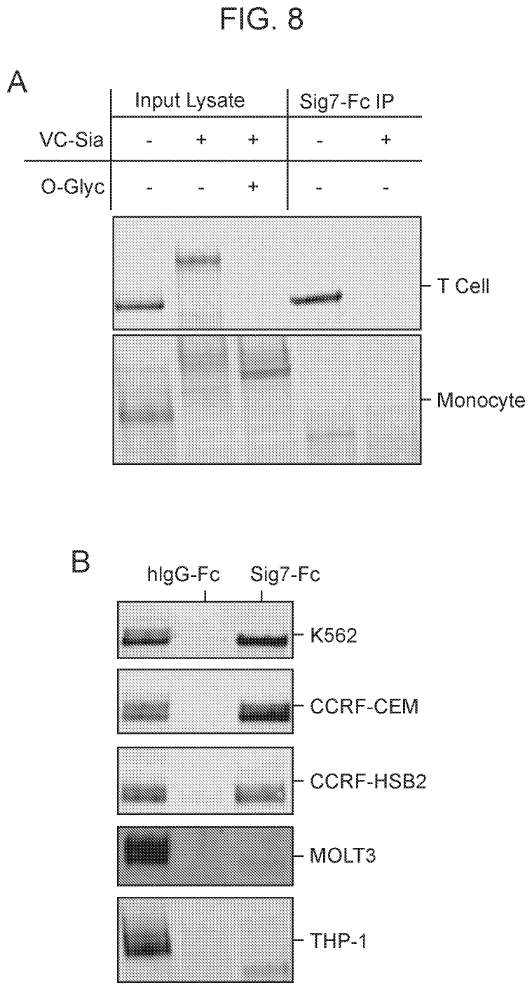

Next, it was determined that Siglec-7 binds a CD43 glycoform containing predominantly disialyl T glycan structures. Results are shown in FIG. 7. Panel A: Pathway for synthesis of sialyl-3-T, sialyl-6-T and disialyl-T O-glycan structures. Bolded genes are glycosyltransferases recovered as significant hits from CRISPRi screen. Panel B: Sequential enzymatic digestion of CD43 with various glycan-degrading enzymes. Sia=VC-Sialidase, β-Gal=β-Galactosidase, O-Glyc=O-Glycosidase from S. Pneumoniae. The latter two enzymes act only on the unsubstituted core 1 structures indicated. Digestions were performed in K562 lysate and CD43 was detected by immunoblot with an antibody against the C-terminal domain. Panel C: Binding of PNA lectin to CD43 treated with glycan-degrading enzymes. Panel D: O-glycomics analysis of CD43 performed by chemical beta-elimination of O-glycans and LC-MS. Panel E: MS/MS analysis of monosialylated glycan structures to determine linkage orientation of sialic acid residues. Panel F: Siglec7-Fc pulldown of CD43 treated with a mucin-specific protease (StcE) to minimally truncate the extracellular domain and VC-Sialidase (Sia) to desialylate the protein. Only full-length CD43 fragments interacted with Siig7-Fc, indicating a binding site close to the N-terminal end of the extracellular domain.

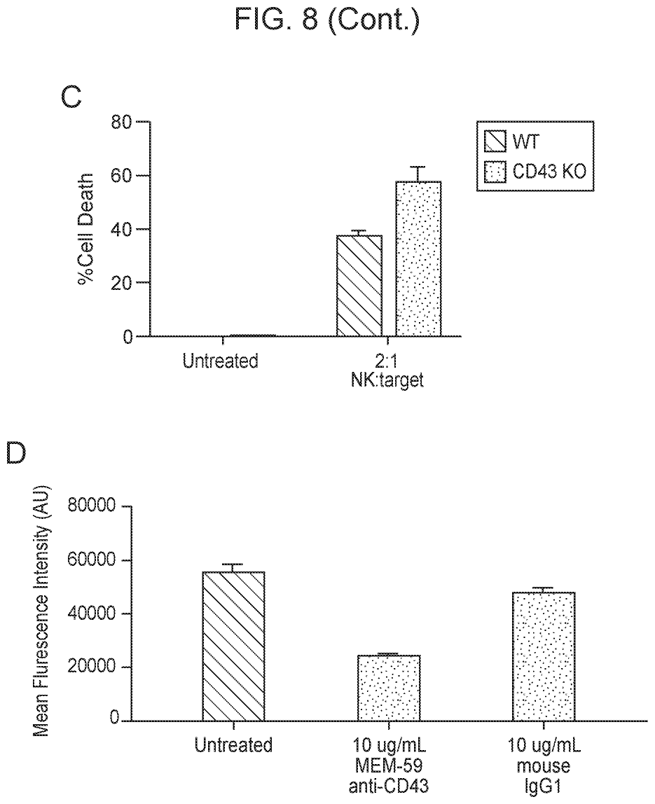

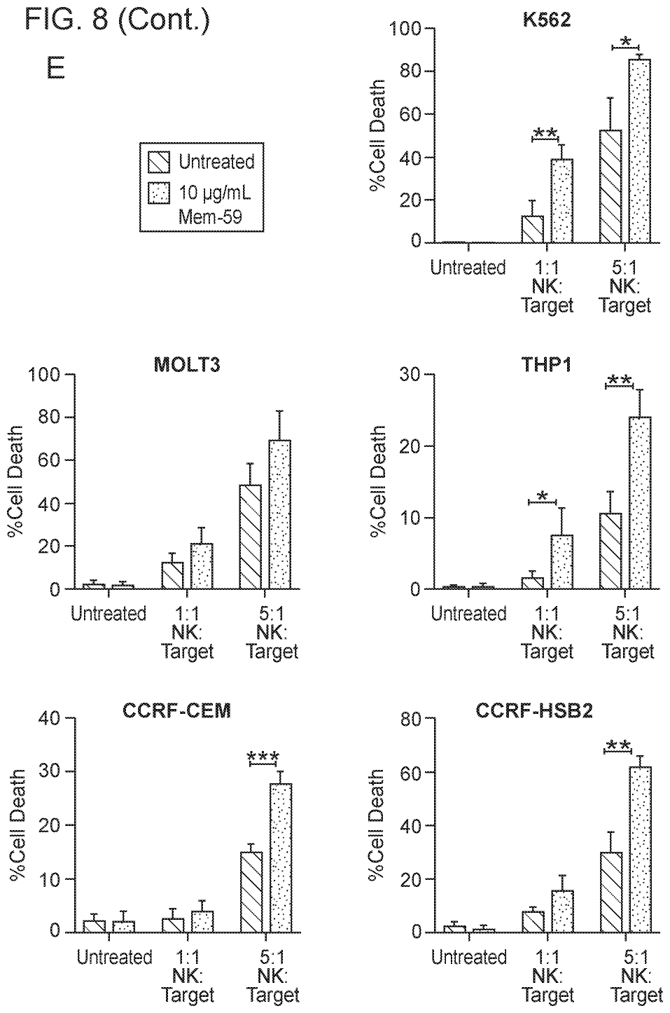

Next, it was determined that the Siglec-7/CD43 interaction suppresses activation of cytotoxic immune cells. Results are shown in FIG. 8. Panel A: Siglec-7-Fc enrichment of T cell and monocyte lysates isolated from primary human PBMCs. Enzyme digestions and CD43 immunoblotting was performed to identify discrete glycoforms bound by Siglec-7. Panel B: Siglec-7 enrichment of lysates prepared from a range of leukemia cell lines shows varying expression of Siglec-7-binding CD43 glycoforms. Panel C: NK cell killing of CD43 KO K562 cells at a 4:1 effector:target ratio. Panel D: Treatment of K562 cells with the Mem-59 anti-CD43 antibody reduces Siglec-7-Fc staining. Panel E: NK cell killing of CD43-expressing leukemic cells following treatment of leukemia cell lines with MEM-59, an anti-CD43 antibody that binds sialylated structures near the CD43 N-terminus. All error bars indicate SEM. *p<0.05, **p<0.01, ***p<0.001.

### Example 3

### ST6GALNAC4 Produces Sialoglycans that Bind to Siglec-E/-7 and Inhibit Macrophage Phagocytosis

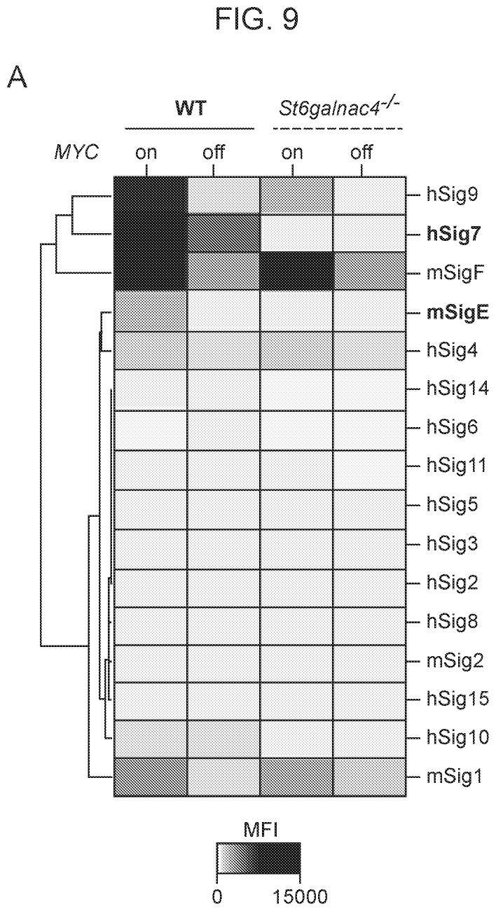

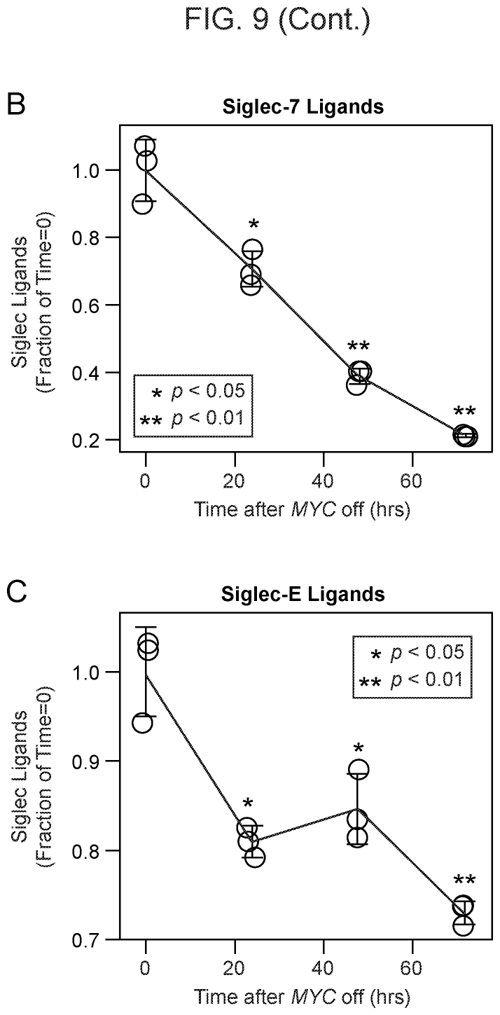

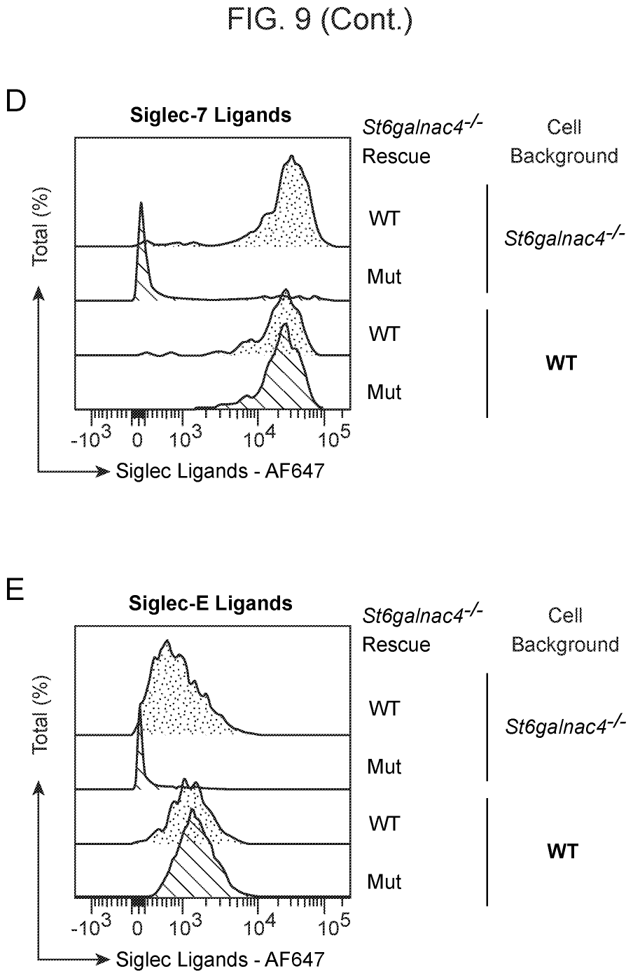

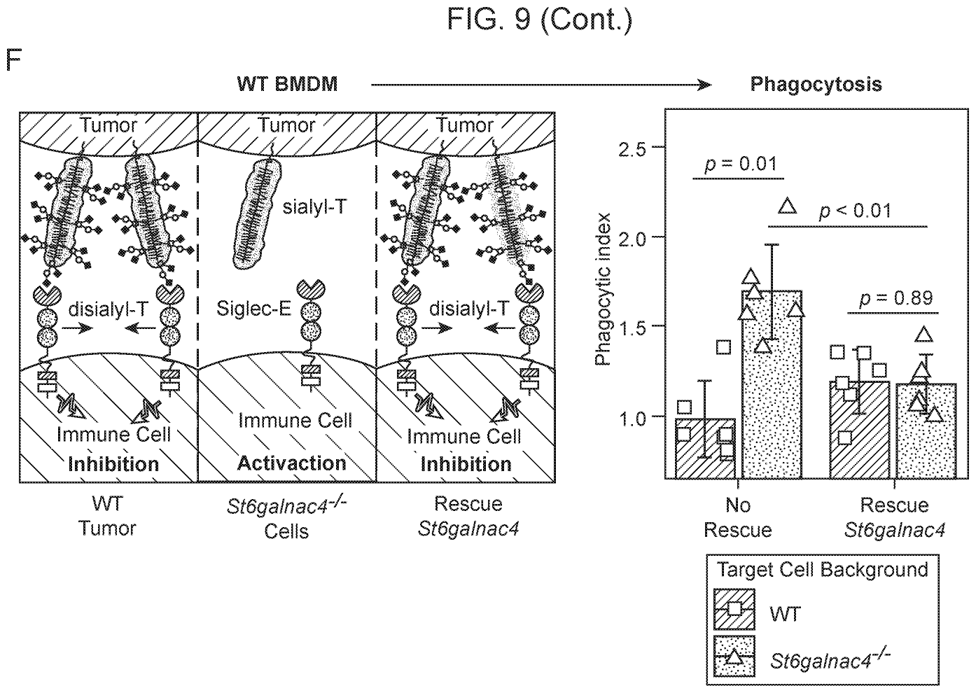

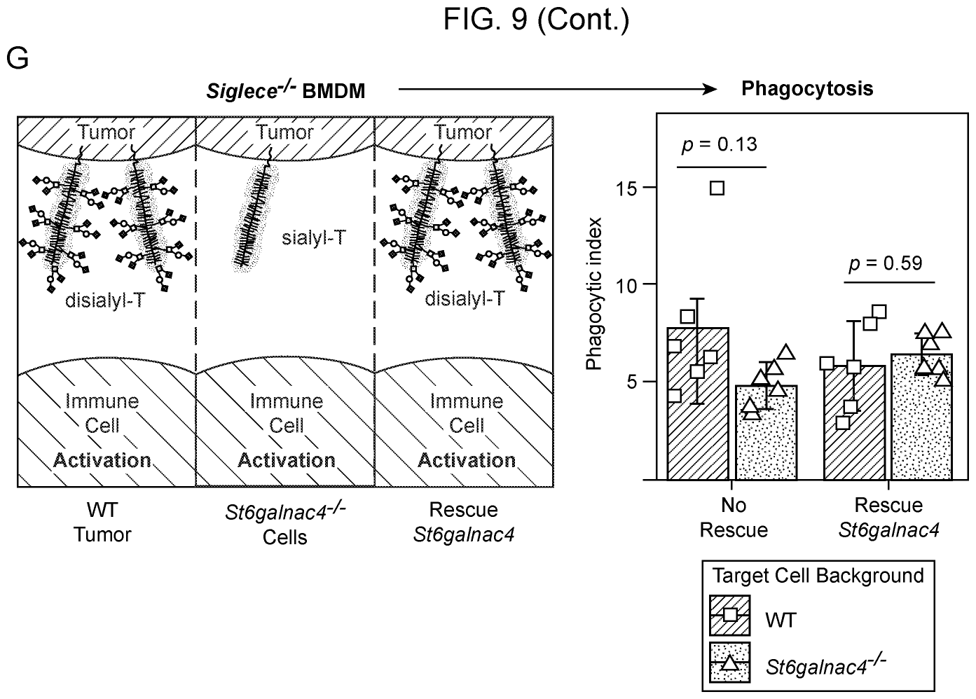

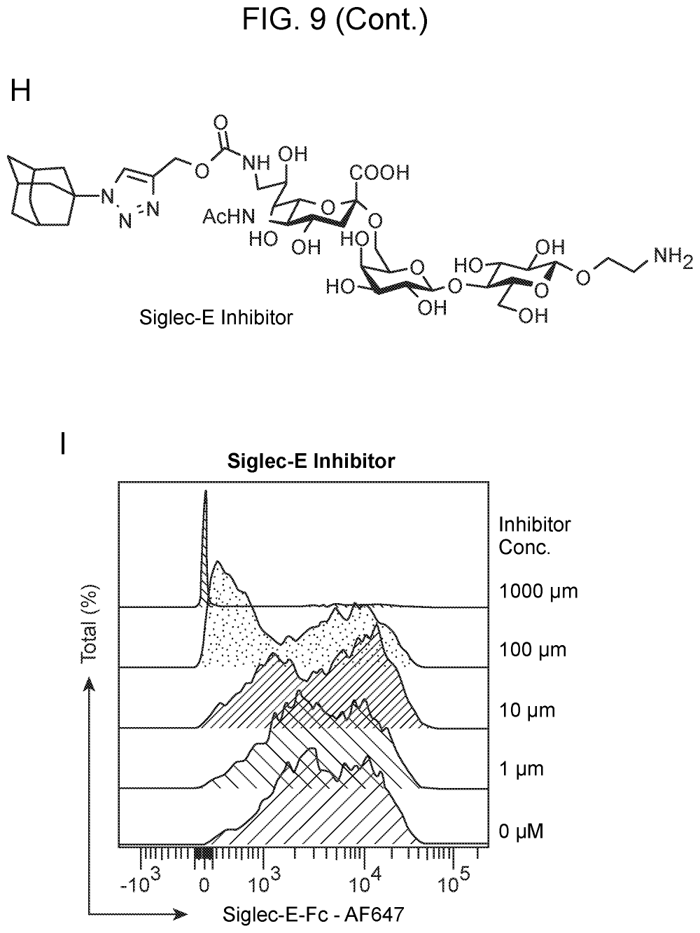

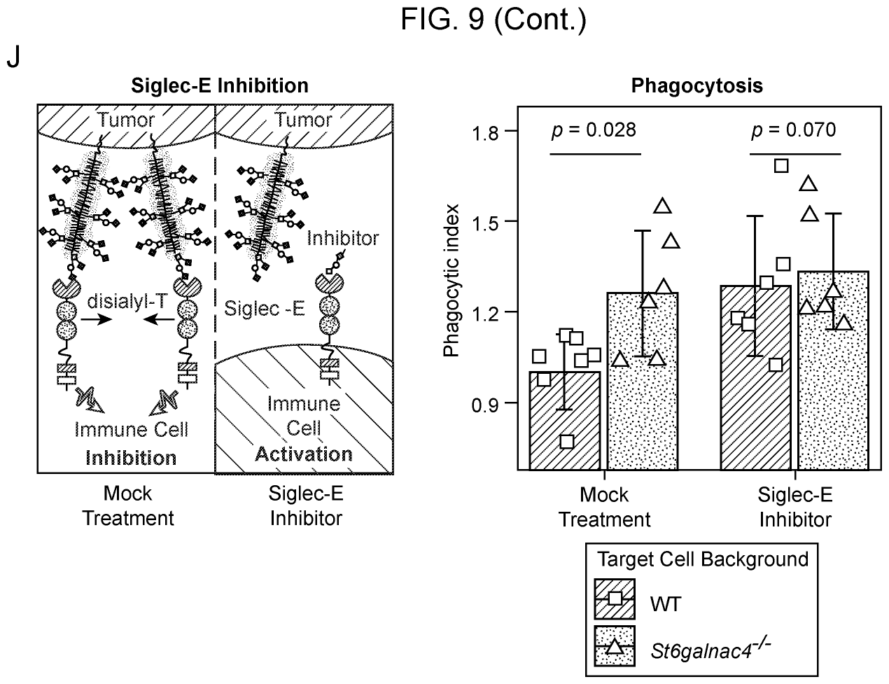

Tumor sialoglycans engage immune cell Siglecs. Assessed herein is whether MYC-promoted disialyl-T engages a Siglec receptor. There are fourteen Siglecs in humans and five homologous proteins in mice. Sialoglycans on T-ALL capable of binding any member of the Siglec family may be detected using Siglec-Fc reagents that comprise the extracellular portion of the Siglec fused to an IgG Fc domain. All commercially available Siglec-Fc reagents were screened against safe targeting control wild type (WT) and St6galnac4^{-/-} T-ALL cells in the MYC on and off states by flow cytometry. Sialoglycan ligands were detected for human Siglecs-7 and -9, and mouse Siglecs-E and -F, on the surface of MYC on WT cells (FIG. 9, panel A). In the MYC off state, display of these Siglec ligands decreased, and ligands for Siglec-E and -7 completely disappeared from St6galnac4^{-/-} cells. Murine Siglec-E is considered the ortholog of human Siglec-7. In MYC off cells, ligands for Siglecs-E and -7 steadily decreased over 72 hours (FIG. 9, panels B and C).

It was confirmed St6galnac4 is sufficient for creation of Siglec ligands by generating WT and St6galnac4^{-/-} cells re-expressing either a mutant St6galnac4 predicted to lack catalytic activity or WT St6galnac4 (data not shown). St6galnac4^{-/-} cells re-expressing WT enzyme recovered the ability to bind Siglecs-7 and -E, while St6galnac4^{-/-} cells expressing the mutant St6galnac4 did not (FIG. 9, panels D and E). Therefore, disialyl-T is produced by St6galnac4 and is a ligand for mouse Siglec-E and human Siglec-7.

Siglec-E has been reported to modulate macrophage activity. Hence, it was assessed whether the presence of disialyl-T, and thus Siglec-E ligands, on T-ALL decreases phagocytosis by macrophages. T-ALL cells that were labeled with a pH-sensitive red dye to detect internalization and entry into the lysosome were co-incubated with bone marrow-derived macrophages (BMDMs). Phagocytosis of St6galnac4^{-/-} cells in the co-culture was higher than of controls (data not shown) and was reversed when the knockout cells were rescued by re-expressing WT St6galnac4 (FIG. 9, panel F). Disialyl-T produced by St6galnac4 inhibits phagocytosis of T-ALL.

To test if Siglec-E influences macrophage phagocytosis, two studies were performed. First, BMDMs from Siglece^{-/-} mice were generated. These Siglece^{-/-} BMDMs phagocytosed tumor cells to a greater extent than their Sig/ece-positive counterparts, but could not discriminate between WT and St6galnac4^{-/-} target cells (FIG. 9, panel G). Second, we synthesized a small molecule selective Siglec-E inhibitor and demonstrated that it blocks binding of Siglec-E-Fc to target cells (FIG. 9, panels H and I). Addition of the Siglec-E inhibitor to co-cultures both increased macrophage phagocytosis and eliminated the ability of macrophages to discriminate between WT and St6galnace4^{-/-} target cells (FIG. 9, panel J). It was concluded that disialyl-T engages Siglec-E and in doing so suppresses macrophage phagocytic activity.

### Example 4

### St6galnac4 Promotes MYC-Driven T-ALL Growth In Vivo

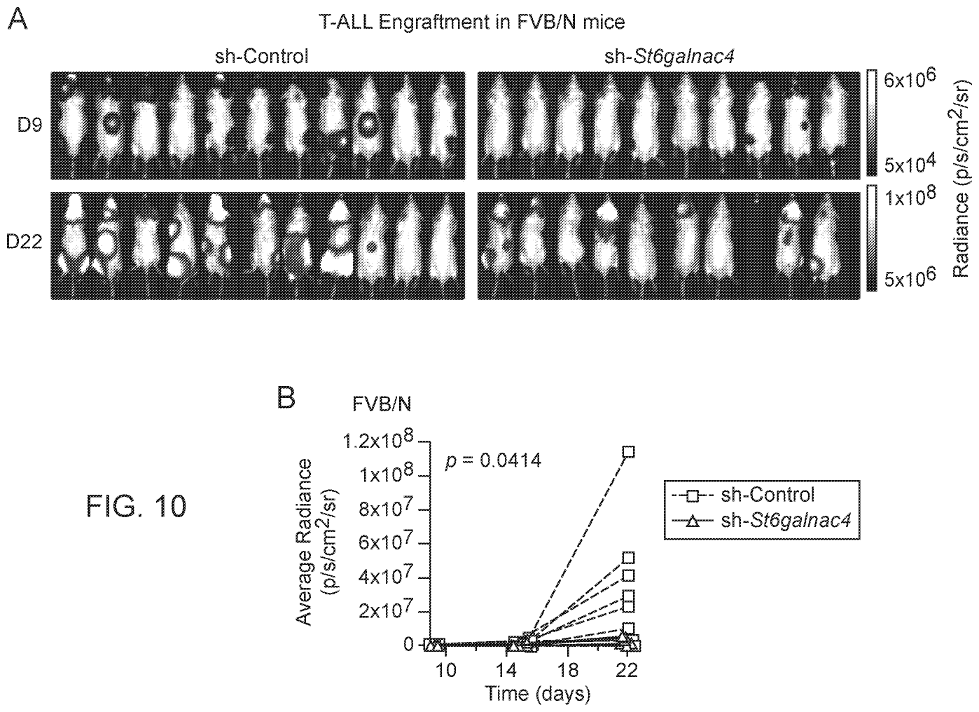

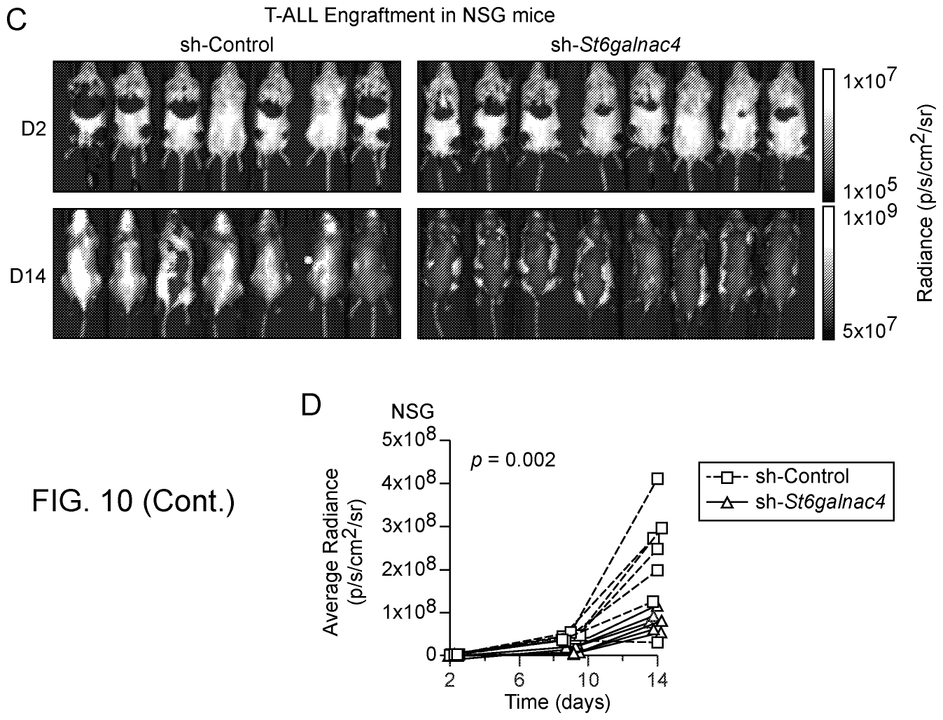

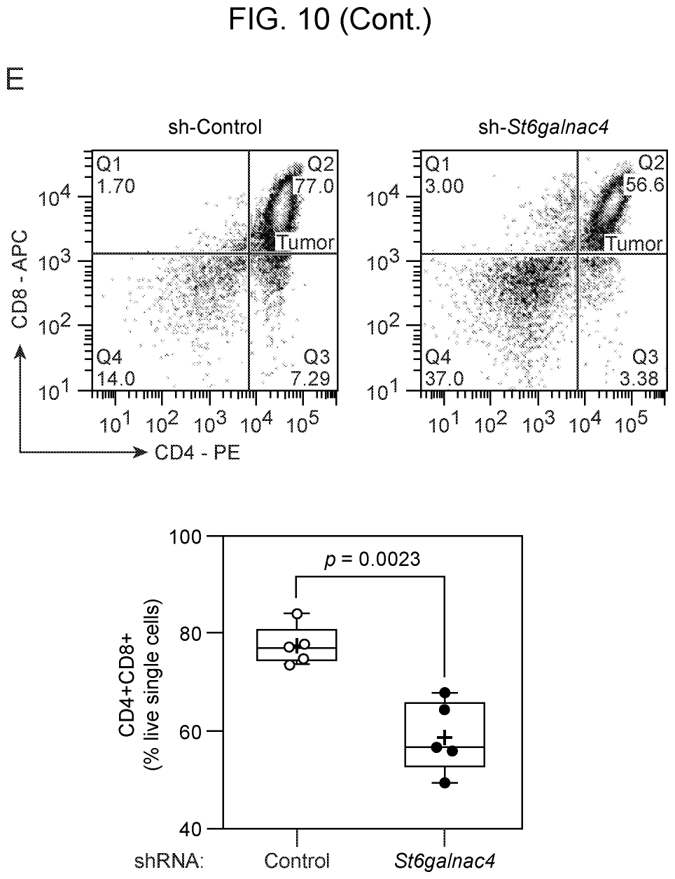

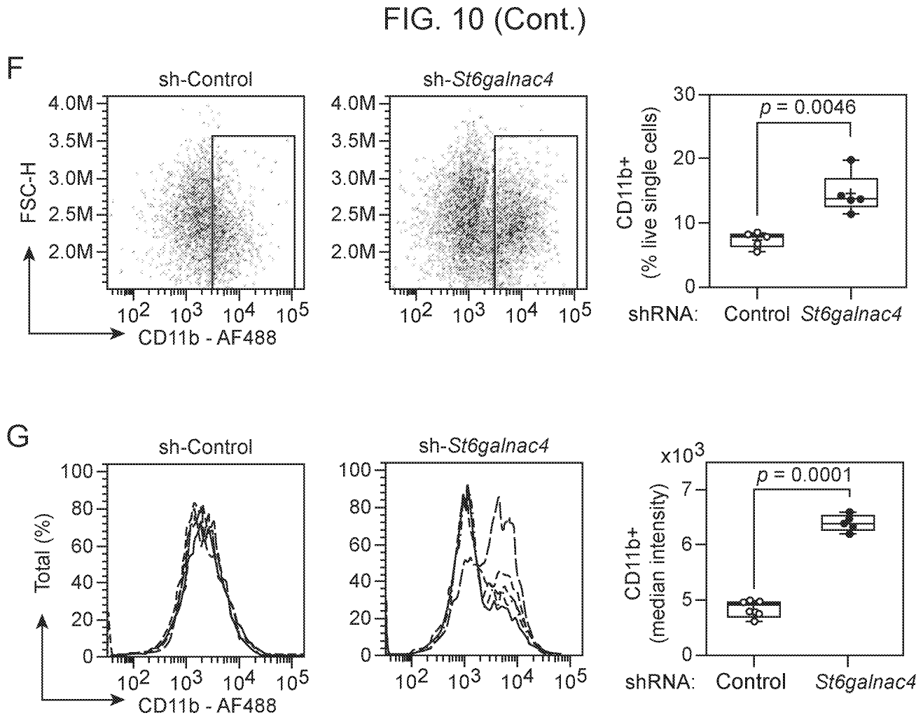

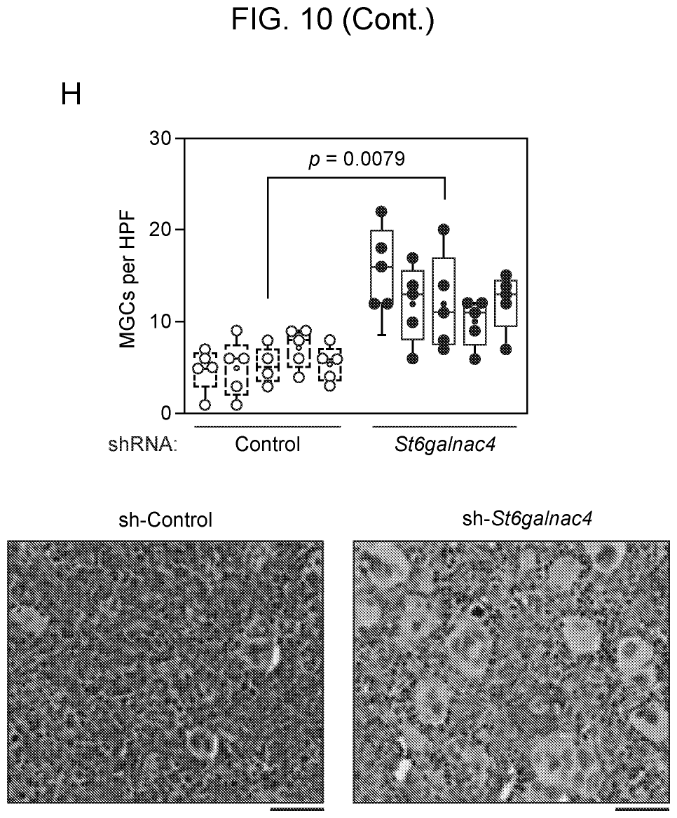

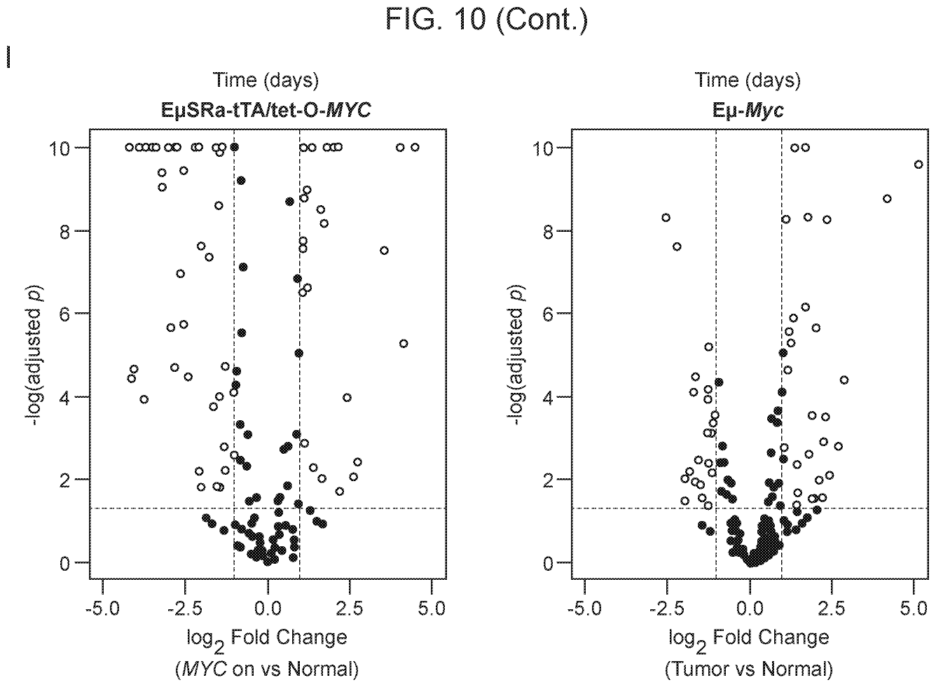

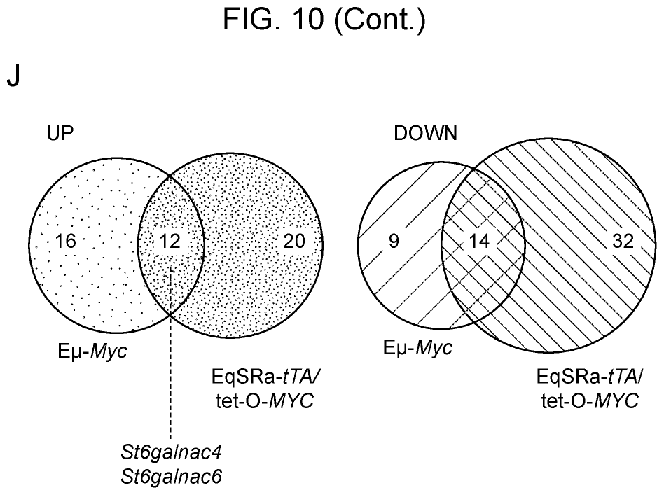

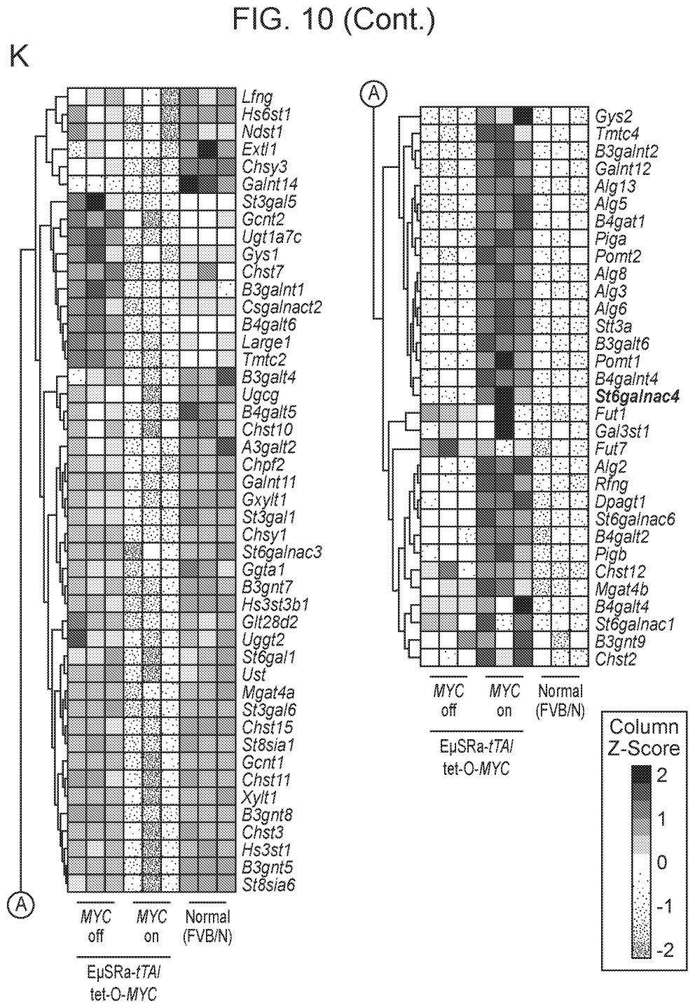

Assessed herein is whether St6galnac4 and disialyl-T promote tumor growth in vivo. T-ALL cells either with or depleted of St6galnac4 expression were tested for their ability to engraft in three different immunological contexts. First, T-ALL St6galnac4 knockdown cells (data not shown) exhibited reduced tumor growth when transplanted into immunocompetent, WT FVB/N mice intravenously (FIG. 10, panels A and B) and subcutaneously (data not shown), while St6galnac4 depletion conferred a minimal in vitro growth advantage compared to control (data not shown). Second, intravenous transplantation of T-ALL into immunocompromised Rag1^{-/-} FVB/N mice showed diminished growth of St6galnac4^{-/-} tumors relative to control (data not shown). Third, intravenous transplantation of T-ALL into immunodeficient NOD/SCID/IL2rg^{-/-} (NSG) mice similarly revealed slowed progression of tumors depleted of St6galnac4 (FIG. 10, panels C and D). These data indicate that St6galnac4 promotes growth and engraftment of T-ALL in vivo.

A difference was observed in the immune response to St6galnac4-depleted tumors that was surprisingly present even in NSG mice, in which B cells, T cells and functional NK cells are absent and antigen-presenting cells are defective. Knockdown of St6galnac4 reduced splenic CD4⁺CD8⁺ T-ALL burden (FIG. 10, panel E) and was accompanied by a relative increase of myeloid cells (CD11b+ cells: 14.5% vs. 7.4% in control) (FIG. 10, panel F). In St6galnac4-depleted tumors, the size of CD11b+ cells was larger (data not shown) and CD11b levels were 30% higher (FIG. 10, panel G), indicating activation of the myeloid compartment⁶⁴. Histologically, we observed an infiltration of giant myeloid cells into tumors depleted of St6galnac4 as compared to control tumors, pointing to a role for St6galnac4 in preventing activation or recruitment of myeloid cells (FIG. 10, panel H). Therefore, St6galnac4 inhibits myeloid-mediated immunity and promotes tumor development in vivo, consistent with our observation of reduced phagocytosis of cells expressing St6galnac4.

Also examined is whether MYC regulates glycosylation in two autochthonous models that mimic in situ tumorigenesis and engraftment. RNA-seq of the Ep-Myc transgenic mouse model of Burkitt's lymphoma and the EpSRa-tTA/tet-O-MYC model of T-ALL highlighted changes to the expression of several glycogenes upon MYC-driven leukemia and lymphoma development (FIG. 10, panel I). Importantly, upregulation of St6galnac4 is a core component of this glycogene signature and was preserved in both types of MYC-driven leukemia and lymphoma (FIG. 10, panel J). The glycogene signature MYC induces upon tumorigenesis is reversible. MYC-directed changes to most glycogenes, including St6galnac4, return to normal levels within four days of T-ALL regression induced upon MYC inactivation (FIG. 10, panel K). It was concluded that MYC regulates the expression of glycosylation machinery in general, and St6galnac4 in particular, during in situ tumorigenesis.

### Example 5

### MYC and ST6GALNAC4 Predict Poor Prognosis in Human Leukemia and Lymphoma

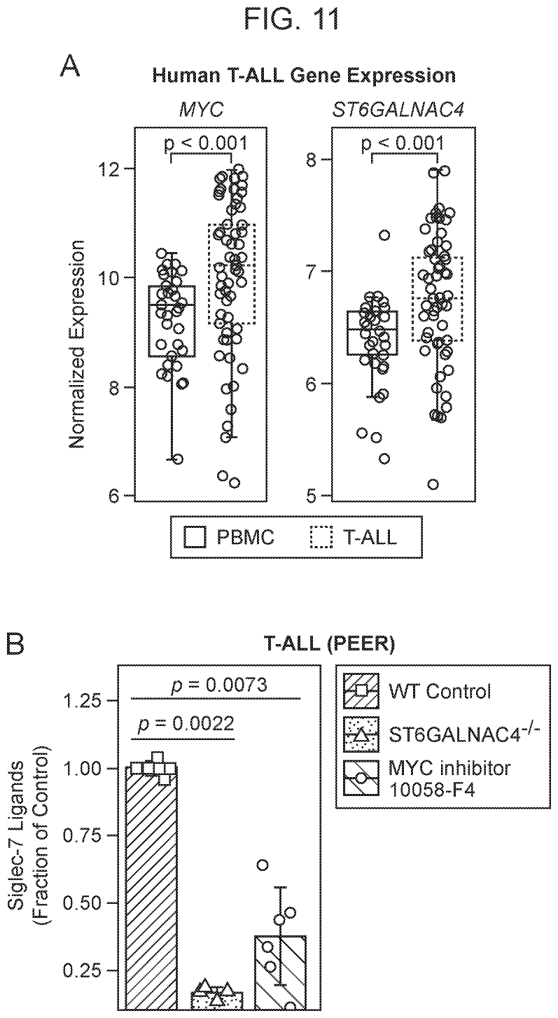

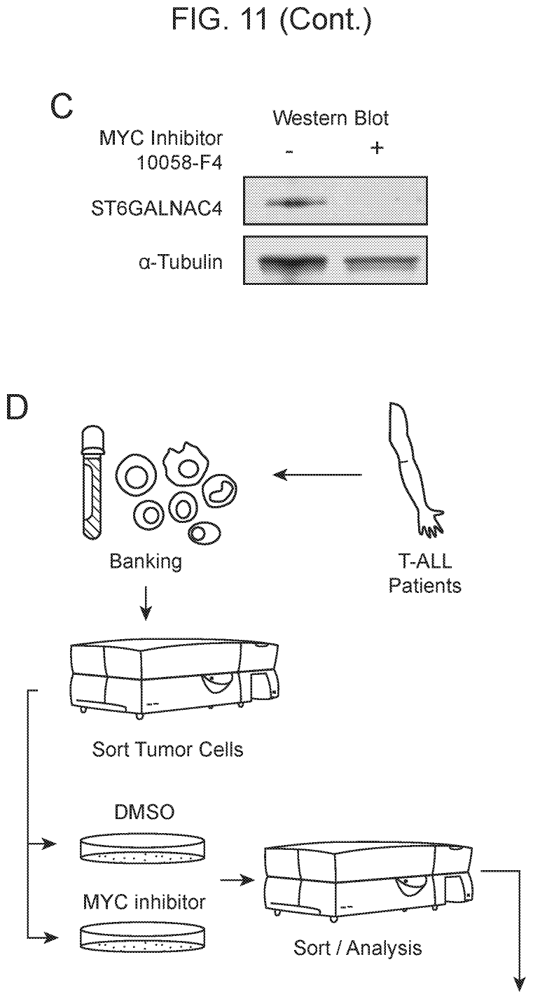

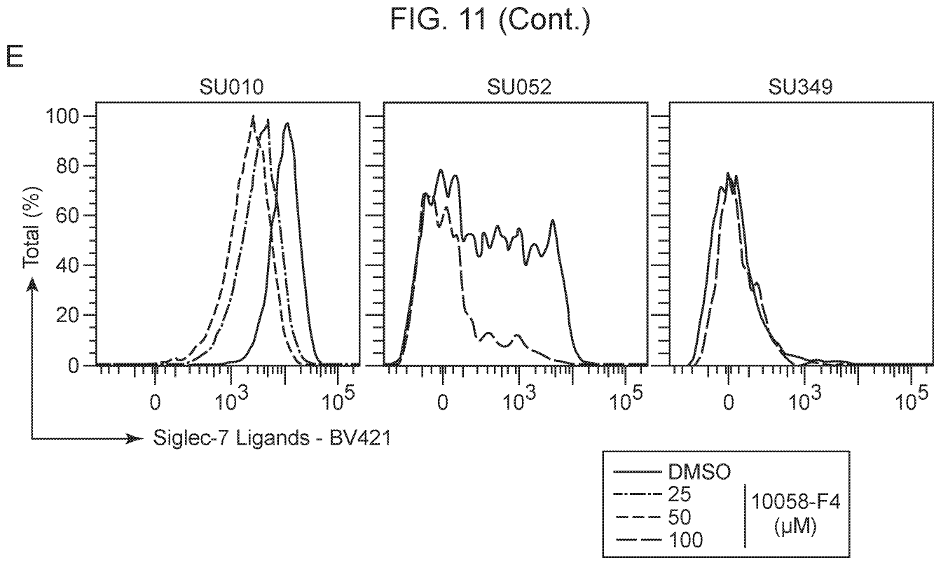

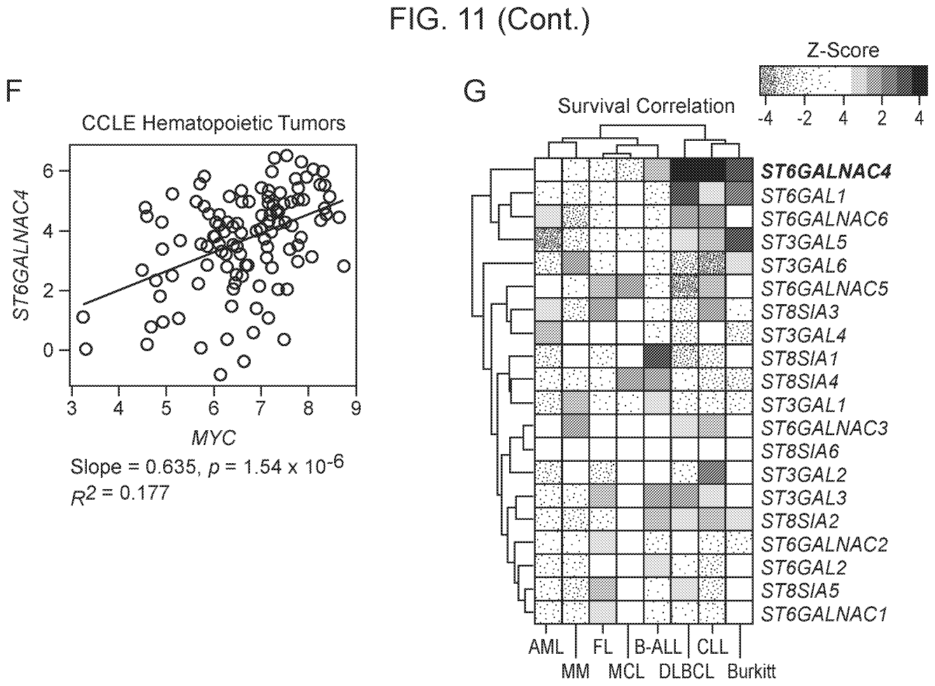

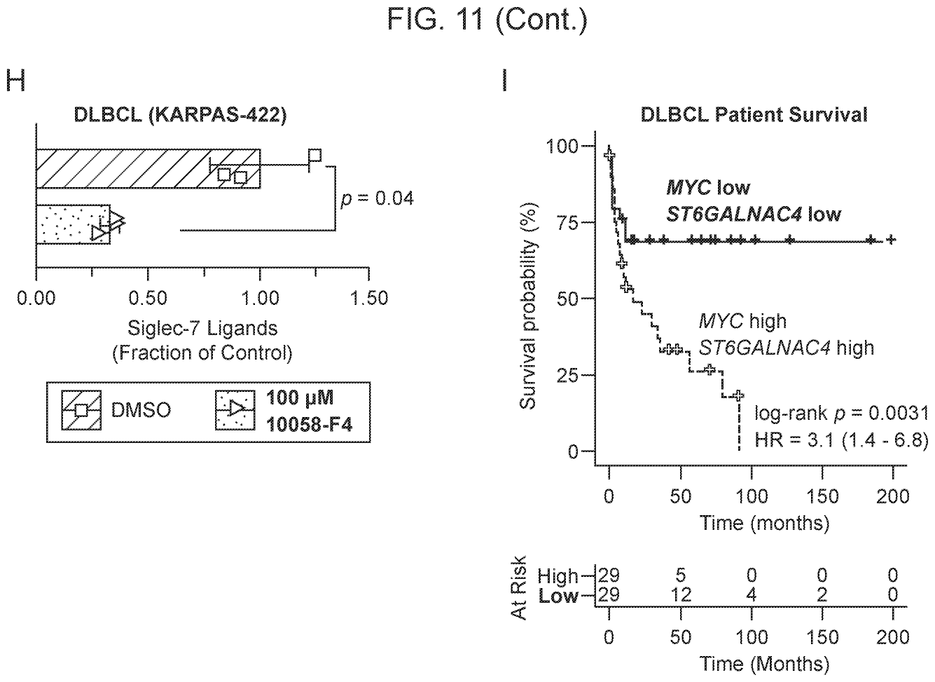

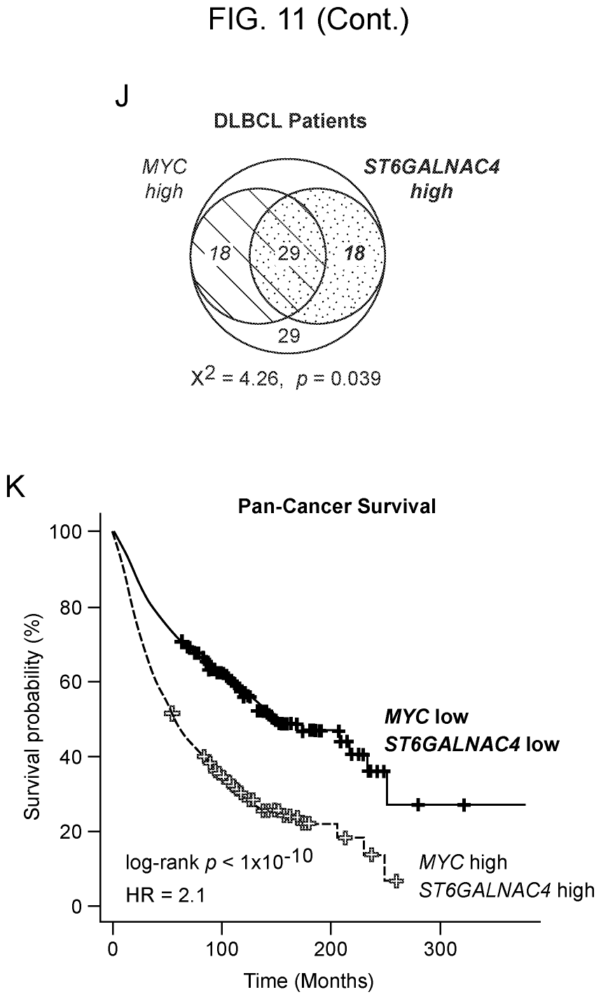

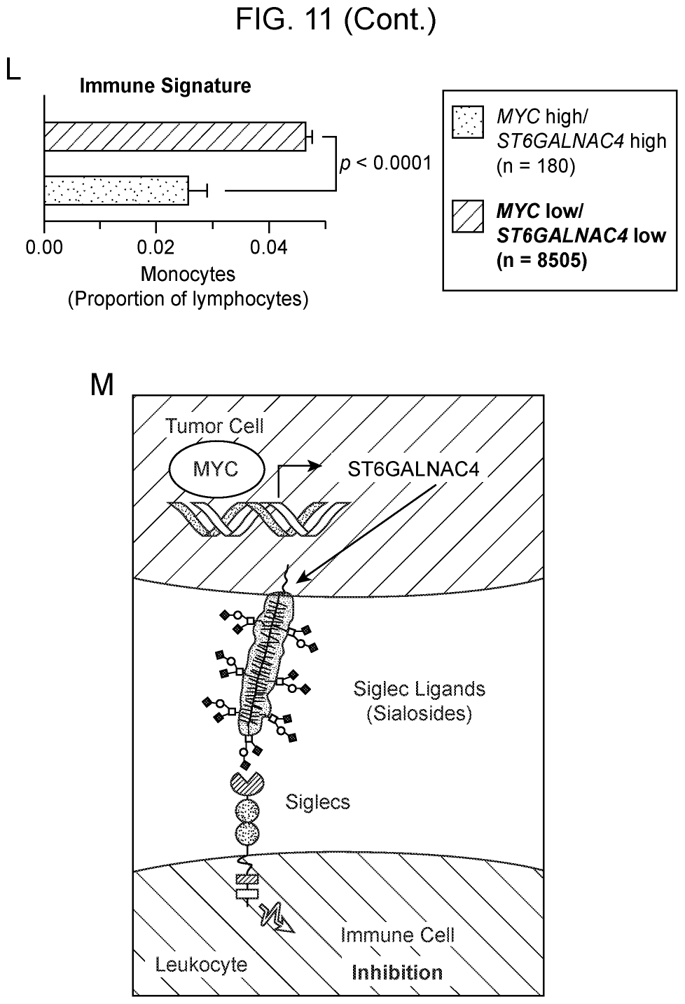

Epistatic activation of MYC drives human T-ALL. We studied whether MYC also controls glycosylation in human T-ALL. By compiling three existing datasets of over 50 patient samples, we confirmed elevated MYC and accompanying ST6GALNAC4 expression in human T-ALL (FIG. 11, panel A). Asked next was whether ST6GALNAC4 synthesizes sialoglycan ligands for human Siglec-7. Knocking out ST6GALNAC4 resulted in a sharp reduction in display of Siglec-7 ligands on the surface of PEER cells, a model human T-ALL cell line (FIG. 11, panel B). Two different inhibitors were then used, 10058-F4, an inhibitor of MYC/MAX dimerization, and THZ1, a CDK7/9 inhibitor with secondary effects on MYC, to ask whether MYC regulates display of Siglec-7 ligands. Pharmacological MYC or CDK7/9 inhibition reduced Siglec-7-Fc binding (FIG. 11, panel B) and was accompanied by diminished ST6GALNAC4 protein expression (FIG. 11, panel C).

Assessed next was whether T-ALL liquid biopsies display Siglec-7 ligands susceptible to MYC regulation. We collected samples from patients presenting to the Stanford Hematology Clinic with new onset T-ALL who were naïve to therapy, and treated FACS-sorted tumor cells with the MYC inhibitor 10058-F4. We detected Siglec-7-Fc binding activity on T-ALL cells from two of three patients, and in both cases, MYC inhibition reduced Siglec-7 ligand display (FIG. 11, panels D and E). Therefore, MYC promotes display of sialoglycans on primary human T-ALL cells that interact with Siglec-7, consistent with our observations in murine cell lines.

To examine if MYC regulates ST6GALNAC4 and Siglec-7 ligand production more generally in hematopoietic tumors, we inspected gene expression data for all hematopoietic tumors contained in the Cancer Cell Line Encyclopedia (CCLE) and found a positive correlation between MYC and ST6GALNAC4 expression (FIG. 11, panel F). Through PRECOG, we found that high ST6GALNAC4 expression, among all sialyltransferases, is the strongest predictor of adverse patient outcomes in diffuse large B cell lymphoma (DLBCL), chronic lymphocytic lymphoma (CLL), and Burkitt lymphoma (BL), which are notably MYC-driven malignancies (FIG. 11, panel G). There were no T-ALL data in the PRECOG dataset. Therefore, we compared the survival covariance of all sialyltransferases and MYC expression across all tumor types in PRECOG. Indeed, MYC and ST6GALNAC4 had a high covariance (data not shown). Thus, patients with high MYC expression are also likely to have high ST6GALNAC4 expression (data not shown).

The causal relationship of MYC and ST6GALNAC4 activity with Siglec ligand production suggests that MYC and ST6GALNAC4 gene expression could serve as a signature of immune inhibitory glycosylation. To test this, we studied DLBCL, an aggressive type of non-Hodgkin lymphoma characterized by high MYC activity. Inhibition of MYC with 10058-F4 caused a 70% reduction in Siglec-7 ligand abundance on KARPAS-422 DLBCL cells (FIG. 11, panel H). In a cohort of DLBCL patients, individuals with combined high MYC and high ST6GALNAC4 expression had strongly decreased overall survival (log-rank p=0.0031) and increased risk of death (HR=3.1, 95% Cl 1.4-6.8) (FIG. 11, panel I). There was no difference in survival among patients lacking this gene signature (log-rank p=0.58; HR=1.3, 95% CI 0.50-3.6) (data not shown). Indeed, patients with either high MYC or high ST6GALNAC4 expression tended to be the same individuals, underscoring the linked effects of these two genes on patient survival (FIG. 11, panel J).

Finally, the consequences of combined overexpression of MYC and ST6GALNAC4 was tested by performing a pan-cancer survival analysis of TCGA data. Across all cancers, high expression of MYC and ST6GALNAC4 together is associated with decreased survival (log-rank p<1×10⁻¹⁰; HR=2.1) (FIG. 11, panel K). Thorsson et al. reported immune profiling data on tumors from patients in the TCGA dataset⁷⁵. We analyzed these data to determine whether the MYC high/ST6GALNAC4 high gene signature relates to immune status. Tumors with high MYC and high ST6GALNAC4 had significantly fewer monocytes than tumors lacking this signature (4.7% vs. 2.6%), consistent with our observation of myeloid cell exclusion by tumors that express St6galnac4 and disialyl-T in vivo (FIG. 11, panel L). These data indicate that combined overexpression of MYC and ST6GALNAC4 forms a signature of malignant glycosylation associated with a defined immune signature. Importantly, pharmacological inhibition of ST6GALNAC4 to block Siglec ligand biosynthesis may be an effective immune therapy strategy for cancers with elevated MYC activity (FIG. 11, panel M).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1.-87. (canceled)

88. A method of enhancing immunogenicity of a cancer comprising dysregulated Myc, comprising:
administering an effective amount of a sialic acid modulator to an individual identified as having a cancer comprising dysregulated Myc.

89. The method according to claim 88, wherein the sialic acid modulator is a Myc inhibitor.

90. The method according to claim 88, wherein the sialic acid modulator is an inhibitor of sialic acid biosynthesis.

91. The method according to claim 90, wherein the inhibitor of sialic acid biosynthesis is a sialyltransferase inhibitor that inhibits a sialyltransferase upregulated by Myc dysregulation.

92. The method according to claim 91, wherein the sialyltransferase inhibitor inhibits St6galnac4.

93. The method according to claim 88, wherein the sialic acid modulator is a sialidase.

94. The method according to claim 88, wherein the sialic acid modulator is a disialyl-T binding agent.

95. The method according to claim 88, wherein the sialic acid modulator is administered to the individual via a parenteral or an intratumoral route of administration.

96. The method according to claim 88, further comprising, prior to the administering, identifying the individual as having a cancer comprising dysregulated Myc.

97. The method according to claim 96, wherein identifying the individual as having a cancer comprising dysregulated Myc comprises assessing the levels of disialyl-T on the surface of cancer cells of the individual.

98. The method according to claim 96, wherein identifying the individual as having a cancer comprising dysregulated Myc comprises assessing the expression level of St6galnac4 in cancer cells of the individual.

99. A method of enhancing immunogenicity of a cancer, comprising:
administering an effective amount of a disialyl-T modulator to an individual identified as having a cancer characterized by cell surface display of disialyl-T.

100. The method according to claim 99, wherein the disialyl-T modulator is an inhibitor of disialyl-T biosynthesis.

101. The method according to claim 100, wherein the inhibitor of disialyl-T biosynthesis is a St6galnac4 inhibitor.

102. The method according to claim 100, wherein the disialyl-T modulator binds to disialyl-T and blocks interaction between disialyl-T and a sialic acid-binding Ig-like lectin (Siglec) receptor.

103. A method of enhancing immunogenicity of a cancer, comprising:
(i) administering an effective amount of an agent to an individual identified as having a cancer comprising cell surface expression of a glycoprotein comprising an O-glycosylated mucin-like domain, wherein the agent modulates the glycoprotein; or
(ii) administering an effective amount of a soluble glycosylated CD43 polypeptide to an individual having cancer, wherein the soluble glycosylated CD43 polypeptide binds to Siglec-7 in the individual.

104. The method according to claim 103(i), wherein the agent is a mucin-degrading agent.

105. The method according to claim 103(i), wherein the agent binds glycosylated CD43 and blocks the interaction between glycosylated CD43 and Siglec-7.

106. The method according to claim 103(ii), wherein the soluble glycosylated CD43 polypeptide comprises disialyl-T.

107. The method according to claim 103(ii), wherein the soluble glycosylated CD43 polypeptide comprises an N-terminal fragment of CD43, wherein the N-terminal fragment comprises amino acids 20-253 of CD43 or a fragment thereof.


SEQUENCE LISTING (1 sequence)

SEQ ID NO: 1 - protein, 400 aa, Homo sapiens
MATLLLLLGVLVVSPDALGSTTAVQTPTSGEPLVSTSEPLSSKMYTTSITSDPKADSTGDQTSALPPSTSINEGSPLWTSIGASTGSPLPEPTTYQEVSIKMSSVPQETPHATSHPAVPITANSLGSHTVTGGTITTNSPETSSRTSGAPVTTAASSLETSRGTSGPPLTMATVSLETSKGTSGPPVTMATDSLETSTGTTGPPVTMTTGSLEPSSGASGPQVSSVKLSTMMSPTTSTNASTVPFRNPDENSRGMLPVAVLVALLAVIVLVALLLLWRRRQKRRTGALVLSRGGKRNGVVDAWAGPAQVPEEGAVTVTVGGSGGDKGSGFPDGEGSSRRPTLTTFFGRRKSRQGSLAMEELKSGSGPSLKGEEEPLVASEDGAVDAPAPDEPEGGDGAAP